SEQUENCE LISTING (4 sequences)

SEQ ID NO: 1 - protein, 22 aa, synthetic construct
YARAAARQARAKALARQLGVAA

SEQ ID NO: 2 - protein, 17 aa, synthetic construct
RRRRRRKALARQLGVAA

SEQ ID NO: 3 - protein, 24 aa, synthetic construct
GRKKRRQRRRPPQKALARQLGVAA

SEQ ID NO: 4 - protein, 38 aa, synthetic construct
GWTLNSAGYLLGKINLKALAALAKKILKALARQLGVAA

(19) United States
(12) Patent Application Publication
(10) Pub. No.: US 2025/0041571 A1
(43) Pub. Date: Feb. 6, 2025

(54) DRUG COATED MEDICAL DEVICES

(71) Applicant: Vanderbilt University, Nashville, TN (US)

(72) Inventors: Craig L. Duvall, Nashville, TN (US); John W. Tierney, Nashville, TN (US)

(21) Appl. No.: 18/790,800

(22) Filed: Jul. 31, 2024

Related U.S. Application Data
(60) Provisional application No. 63/516,746, filed on Jul. 31, 2023.

(51) Int. Cl. A61M 25/10; A61K 45/06

(52) U.S. Cl. CPC A61M 25/104; A61K 45/06; A61M 25/1027; A61M 2025/1031; A61M 2205/0238; A61M 2207/10; A61M 2210/12

(57) ABSTRACT

Disclosed herein are drug coated medical devices that can be used for the treatment of vascular diseases. An example device includes a medical device having an outer surface, and a multi-layer drug coating on the outer surface, the multi-layer drug coating including at least one polycationic layer including a cationic therapeutic and at least one polyanionic layer including an anionic polymer. Also disclosed are methods of making the drug coated medical devices and methods of treating vascular diseases.

### CROSS-REFERENCE TO RELATED APPLICATIONS

This application claims priority to U.S. Provisional Patent Application No. 63/516,746 filed on Jul. 31, 2023, which is incorporated fully herein by reference.

### STATEMENT REGARDING FEDERALLY SPONSORED RESEARCH

This invention was made with Government support under Federal Grant no. F31HL162476 awarded by the National Heart, Lung, and Blood Institute. The Federal Government has certain rights to this invention.

### REFERENCE TO AN ELECTRONIC SEQUENCE LISTING

The contents of the electronic sequence listing (093386-0028-US02 Sequence Listing.xml; Size: 5,459 bytes; and Date of Creation: Jul. 31, 2024) is herein incorporated by reference in its entirety.

### TECHNICAL FIELD

This disclosure relates to drug coated medical devices, including angioplasty balloon catheters, and their use in biomedical applications.

### INTRODUCTION

Devices and methods that can aid in the prevention of restenosis can be useful in the treatment of numerous vascular diseases.

### SUMMARY

In one aspect, disclosed are drug coated medical devices comprising: a medical device having an outer surface; and a multi-layer drug coating on at least a portion of the outer surface, the multi-layer drug coating comprising at least one polyelectrolyte bilayer, the polyelectrolyte bilayer comprising a polycationic layer including a cationic therapeutic, and a polyanionic layer capable of electrostatically binding to the polycationic layer, the polyanionic layer including an anionic polymer having recurring units of formula (I)

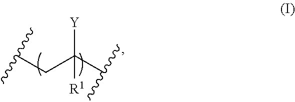

wherein Y is —C(O)OH, —SO₃H, or —PO₃H₂, and R¹ is C₁-C₁₂ alkyl.

In another aspect, disclosed are methods of making a drug coated medical device, the method comprising: (a) immersing an outer surface of a medical device into a first mixture, the first mixture comprising a first solvent and an anionic polymer having recurring units of formula (I)

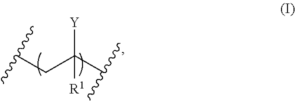

wherein Y is —C(O)OH, —SO₃H, or —PO₃H₂, and R¹ is C₁-C₁₂ alkyl, to provide a polyanionic layer on the outer surface of the medical device, wherein the polyanionic layer includes the anionic polymer; (b) immersing the outer surface of the medical device into a second mixture, the second mixture comprising a second solvent and a cationic therapeutic, to provide a polycationic layer on a surface of the polyanionic layer, wherein the polycationic layer includes the cationic therapeutic; and (c) optionally repeating steps (a) and (b).

In another aspect, disclosed are methods of treating a vascular disease in a subject in need thereof, the method comprising implanting a disclosed drug coated medical device at a vascular site of the subject.

In another aspect, disclosed are methods of delivering a cationic therapeutic to a subject in need thereof, the method comprising: (a) implanting a disclosed drug coated angioplasty balloon catheter at a vascular site of the subject; (b) inflating the balloon of the medical device, thereby transferring a portion of the cationic therapeutic from the medical device to the vascular site; (c) deflating the balloon of the medical device; (d) optionally repeating steps (b) and (c); and (e) removing the drug coated medical device from the vascular site.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows a schematic of an example angioplasty balloon catheter application.

FIG. 2 shows a schematic of the MK2 signaling pathway.

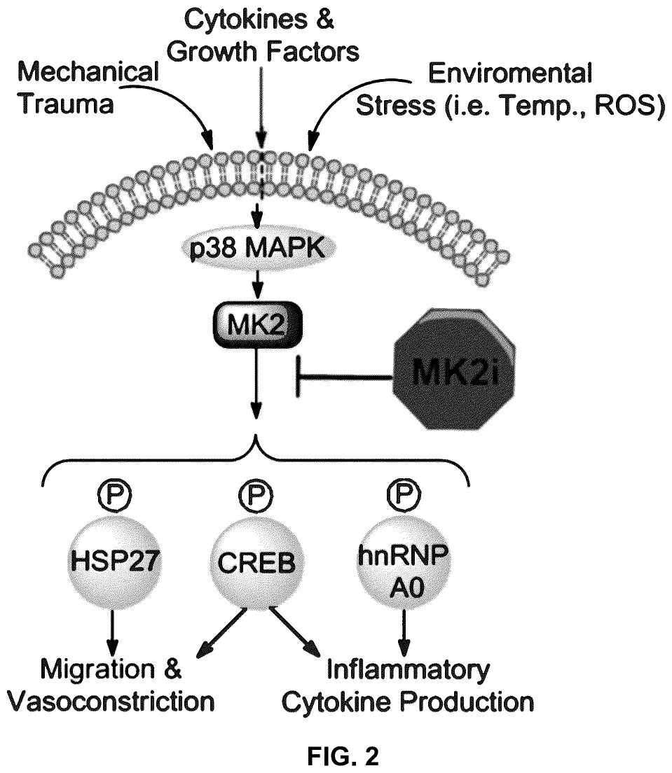

FIG. 3 shows a schematic of an example anionic polymer and an example cationic peptide.

FIG. 4 shows an example method of coating an angioplasty balloon catheter.

FIG. 5A shows a schematic of balloon release analysis.

FIG. 5B shows amounts of MK2i loaded on example balloons.

FIG. 5C shows amounts of MK2i released from example balloons.

FIG. 6A shows fluorescence before and after inflation using In Vivo Imaging System (IVIS) imaging without lactosucrose (LS—top) and 15 mg/mL LS (bottom).

FIG. 6B shows fluorescence before and after inflation using IVIS imaging with LS.

FIG. 6C shows quantification of regions of interests (ROIs) for different example coating formulations.

FIG. 7A shows a schematic of example coated balloons compared to a control method of soaking tissue in a bath of MK2i-nanoparticles (NPs).

FIG. 7B shows a graph of cell retention of example coated balloons and MK2i-NP treatments.

FIG. 7C shows a graph of uptake of example coated balloons and MK2i-NP treatments.

FIG. 8 shows a schematic of an experimental set-up for rat aortae as a model tissue to study drug transfer into the arterial wall.

FIG. 9A shows confocal fluorescence microscopy of ex vivo aortas treated with example balloons without LS.

FIG. 9B shows confocal fluorescence microscopy of ex vivo aortas treated with example balloons with LS.

FIG. 9C shows confocal fluorescence microscopy of ex vivo aortas treated with example balloons with LS.

FIG. 10 shows cellular uptake of poly(propylacrylic acid) (PPAA)-based coatings v. poly(acrylic acid) (PAA)-based coatings.

FIG. 11A shows loading of PPAA-based coatings v. PAA-based coatings.

FIG. 11B shows loading of PPAA-based coatings v. PAA-based coatings.

FIG. 11C shows percent release of PPAA-based coatings v. PAA-based coatings.

FIG. 12 shows visualization of fluorescent MK2i loading with addition of successive layers.

FIG. 13A shows a comparison of MK2i release for layer-by-layer (LbL) conditions using varied number of layers and concentration of coating solution. Significance determined using two-way ANOVA. N=6.

FIG. 13B shows a comparison of MK2i drug loading for LbL conditions using varied number of layers and concentration of coating solution. Significance determined using two-way ANOVA. N=6.

FIG. 14A shows confocal fluorescent microcopy visualization of MK2i transfer from LBL coated balloons to ex vivo rat aorta immediately after balloon inflation for 6 layer, 100 μM MK2i, 10 μM PPAA (6L-100M-10P) balloons without LS. “L” indicates the lumen side of each vessel.

FIG. 14B shows confocal fluorescent microcopy visualization of MK2i transfer from LBL coated balloons to ex vivo rat aorta immediately after balloon inflation for 6 layer, 100 μM MK2i, 10 μM PPAA (6L-100M-10P) balloons with LS. “L” indicates the lumen side of each vessel.

FIG. 15A shows quantification of overall fluorescent MK2i delivery to vessel wall by 6L-100M-10P±LS balloons. Significance was determined using one-way ANOVA with Tukey's post-hoc test. N≥5.

FIG. 15B shows average intensity profile of fluorescent MK2i as a function of depth into the vessel wall. Significance was determined using one-way ANOVA with Tukey's post-hoc test. N≥5.

FIG. 16 shows quantification of MK2i tissue concentration in rat aortas immediately after ex vivo treatment with MK2i-DCBs. N=6.

FIG. 17 shows quantification of MK2i tissue concentration in rat aortas at 24 h after each treatment.

FIG. 18 shows quantification of MK2i tissue concentration in rat aortas at 1-, 3-, and 7-days post-treatment for MK2i-DCBs+LS.

FIG. 19A shows confocal microscope images of fluorescent MK2i treated (without LS) rat aortas after 24 h in the flow loop bioreactor. “L” indicates the lumen side of each vessel.

FIG. 19B shows confocal microscope images of fluorescent MK2i treated (with LS) rat aortas after 24 h in the flow loop bioreactor. “L” indicates the lumen side of each vessel.

FIG. 19C shows confocal microscope images of fluorescent untreated control rat aortas after 24 h in the flow loop bioreactor. “L” indicates the lumen side of each vessel.

FIG. 20A: shows confocal microscopy images of fluorescent MK2i retention after 3 d incubation and 7 d incubation for MK2i-DCBs+LS.

FIG. 20B: shows confocal microscopy images of fluorescent MK2i retention after 3 d incubation and 7 d incubation for untreated controls.

FIG. 21 shows MK2i DCB administration blocks balloon damage induced CREB phosphorylation. P-CREB induction was quantified with undamaged arteries serving as a baseline and balloon-damaged arteries without MK2i treatment representing 100% pCREB induction.

FIG. 22A shows 1 hour P-CREB induction for control aortas. N≥6.

FIG. 22B shows 1 hour P-CREB induction for MK2i+LS coated balloons. N≥6.

FIG. 23 shows pharmacodynamics of P-CREB inhibition over 7 days post-treatment. Half of each aorta was left unstimulated, and the other half was stimulated with LPA for 2 hours to activate CREB. The relative amount of LPA-stimulated P-CREB was measured and compared across groups. N=3.

FIG. 24A shows representative western blot images from P-CREB pharmacodynamics on day 1.

FIG. 24B shows representative western blot images from P-CREB pharmacodynamics on day 3.

FIG. 24C shows representative western blot images from P-CREB pharmacodynamics on day 7. LPA stimulates P-CREB in the untreated, balloon damaged samples, but MK2i DCB inhibits P-CREB activation 7 days after treatment.

FIG. 25A shows confocal fluorescence microscopy of an uninjured control aorta. “L” indicates the lumen side of each vessel.

FIG. 25B shows confocal fluorescence microscopy of untreated and balloon coated treated aortas cultured under flow in the bioreactor for 7 d following balloon injury and stained with PCNA to quantify cellular proliferation. “L” indicates the lumen side of each vessel.

FIG. 26 shows intravascular MK2i-DCB administration can inhibit proliferation. The percentage of PCNA positive cells in the media layer was calculated for each sample. N=6 for untreated and treated groups, N=4 for control groups. The p-values were determined using one-way ANOVA with Tukey's post hoc test.

FIG. 27A shows confocal fluorescence microscopy of an uninjured control aorta. “L” indicates the lumen side of each vessel.

FIG. 27B shows confocal fluorescence microscopy of aortas cultured under flow in the bioreactor for 7 d following balloon injury and stained for fibronectin to quantify cellular ECM production. “L” indicates the lumen side of each vessel.

FIG. 28 shows intravascular MK2i-DCB administration can inhibit ECM production. Fibronectin positive area was normalized to number of nuclei for the media layer of each sample. N=6 for untreated and treated groups, N=4 for control groups. The p-values were determined using one-way ANOVA with Tukey's post hoc test.

FIG. 29A shows confocal fluorescence microscopy images of uninjured control rat ex vivo aortas. Immunohistochemistry (IHC) was performed for contractile proteins SM22 and MYH in aortas maintained under flow culture for 7 d following balloon injury. “L” indicates the lumen side for each vessel.

FIG. 29B shows confocal fluorescence microscopy images of ex vivo rat aortas that were untreated and treated with drug coated balloons (DCBs). IHC was performed for contractile proteins SM22 and MYH in aortas maintained under flow culture for 7 d following balloon injury. “L” indicates the lumen side for each vessel.

FIG. 30A is a graph showing SM22 positive area was normalized to number of nuclei for each sample. The p-values were determined using ANOVA and Tukey's post hoc test. N=6 for untreated and treated groups, N=4 for control groups.

FIG. 30B is a graph showing MYH positive area normalized to number of nuclei for each sample. The p-values were determined using ANOVA and Tukey's post hoc test. N=6 for untreated and treated groups, N=4 for control groups.

FIG. 31 shows a schematic of a muscle bath used for contractility tests performed after culture under flow for 3 d post-injury. The aortas were connected to a force sensor that allowed for measurement of chemically stimulated VSMC contraction.

FIG. 32 shows percent vessel contraction measured in response to increasing concentrations of PE. Statistical significance was determined using an ANOVA and Tukey's post hoc test on IC50 values determined from linear regression models of the PE vs contraction curves. N=3. ***=p<0.005 relative to Control, MK2i DCB, and MK2i NPs.

FIG. 33 shows a schematic of a rat carotid artery balloon injury model. Rat carotids were balloon damaged and either left untreated or treated with MK2i DCB. Carotids were then harvested 14 d after injury for analysis.

FIG. 34A shows trichrome staining of an uninjured rat carotid artery. “L” indicates lumen side for each vessel.

FIG. 34B shows trichrome staining of a rat carotid artery untreated 14 d after injury, with red bar indicating neointimal development. “L” indicates lumen side for each vessel.

FIG. 34C shows trichrome staining of a rat carotid artery treated with a drug coated balloon 14 d after injury, with red bar indicating neointimal development. “L” indicates lumen side for each vessel.

FIG. 35A shows quantification of thickness of intima of carotid arteries 14 days after balloon injury. MK2i DCB treatment effectively inhibits IH development. The p-values were determined with one-way ANOVA and Tukey's post hoc test. N=4 for untreated and MK2i DCB groups, N=8 for undamaged groups.

FIG. 35B shows quantification of intima to media ratio of carotid arteries 14 days after balloon injury. MK2i DCB treatment effectively inhibits IH development. The p-values were determined with one-way ANOVA and Tukey's post hoc test. N=4 for untreated and MK2i DCB groups, N=8 for undamaged groups.

FIG. 36A shows confocal fluorescence microscopy with CD31 staining of uninjured carotid sections.

FIG. 36B shows confocal fluorescence microscopy with CD31 staining of untreated control carotid sections 14 d after surgery.

FIG. 36C shows confocal fluorescence microscopy with CD31 staining of carotid sections confirming vessel endothelial coverage 14 d after surgery. MK2i DCB does not impair endothelial repair.

FIG. 37 shows quantification of endothelial cell coverage from CD31 stained samples. The p-values were determined with one-way ANOVA and Tukey's post hoc test. N=4 for untreated and MK2i DCB groups, N=8 for undamaged groups.

FIG. 38A shows confocal fluorescence staining of an uninjured rat carotid artery for CD68+ macrophages.

FIG. 38B shows confocal fluorescence staining of an untreated rat carotid artery 14 d after injury for CD68+ macrophages.

FIG. 38C shows confocal fluorescence staining of a drug coated balloon treated rat carotid artery 14 d after injury for CD68+ macrophages.

FIG. 39 shows quantification of CD68+ macrophages in the cross-sections of treated or untreated rat carotid arteries. The p-values were determined with one-way ANOVA and Tukey's post hoc test. N=4 for untreated and MK2i DCB groups, N=8 for undamaged groups.

FIG. 40A shows confocal fluorescence staining of an uninjured rat carotid artery for CD3+ T cells.

FIG. 40B shows confocal fluorescence staining of an untreated rat carotid artery 14 d after injury for CD3+ T cells.

FIG. 40C shows confocal fluorescence staining of a drug coated balloon treated rat carotid artery 14 d after injury for CD3+ T cells.

FIG. 41 shows quantification of CD3+ T cells in rat carotid arteries 14 d after injury. The p-values were determined with one-way ANOVA and Tukey's post hoc test. N=4 for untreated and MK2i DCB groups, N=8 for undamaged groups.

FIG. 42A shows confocal fluorescence microscopy of an uninjured rat carotid artery stained with PCNA.

FIG. 42B shows confocal fluorescence microscopy of an untreated rat carotid artery 14 d after injury stained with PCNA.

FIG. 42C shows confocal fluorescence microscopy of a drug coated balloon treated rat carotid artery 14 d after injury stained with PCNA.

FIG. 43 shows percentage of proliferating cells quantified for rat carotid artery samples taken 14 d after damage. The p-values were determined with one-way ANOVA and Tukey's post hoc test. N=4 for untreated and MK2i DCB groups, N=8 for undamaged groups.

FIG. 44A shows confocal fluorescence microscopy of an uninjured rat carotid artery stained for fibronectin.

FIG. 44B shows confocal fluorescence microscopy of an untreated rat carotid artery 14 d after injury stained for fibronectin.

FIG. 44C shows confocal fluorescence microscopy of a drug coated balloon treated rat carotid artery 14 d after injury stained for fibronectin.

FIG. 45 shows quantification of fibronectin deposition for rat carotid artery samples taken 14 d after damage. The p-values were determined with one-way ANOVA and Tukey's post hoc test. N=4 for untreated and MK2i DCB groups, N=8 for undamaged groups.

FIG. 46A shows confocal fluorescence microscopy of an uninjured rat carotid artery stained for vimentin, a characteristic protein of the synthetic VSMC phenotype, to evaluate VSMC phenotype switching.

FIG. 46B shows confocal fluorescence microscopy of an untreated rat carotid artery 14 d after injury stained for vimentin.

FIG. 46C shows confocal fluorescence microscopy of a drug coated balloon treated rat carotid artery 14 d after injury stained for vimentin.

FIG. 47 shows quantification of vimentin for rat carotid artery samples taken 14 d after damage. The p-values were determined with one-way ANOVA and Tukey's post hoc test. N=4 for untreated and MK2i DCB groups, N=8 for undamaged groups.

FIG. 48A shows confocal fluorescence microscopy of an uninjured rat carotid artery stained for SM22 to assess the preservation of the healthy contractile VSMC phenotype.

FIG. 48B shows confocal fluorescence microscopy of an untreated rat carotid artery 14 d after injury stained for SM22 to assess the preservation of the healthy contractile VSMC phenotype.

FIG. 48C shows confocal fluorescence microscopy of a drug coated balloon treated rat carotid artery 14 d after injury stained for SM22 to assess the preservation of the healthy contractile VSMC phenotype.

FIG. 49 shows quantification of SM22 for rat carotid artery samples taken 14 d after damage. The p-values were determined with one-way ANOVA and Tukey's post hoc test. N=4 for untreated and MK2i DCB groups, N=8 for undamaged groups.

FIG. 50 is a confocal fluorescence microscopy image showing retention of MK2i in MK2i DCB treated carotid artery after 14 d.

FIG. 51A shows Gomori trichrome staining of undamaged rat carotid arteries.

FIG. 51B shows Gomori trichrome staining of untreated rat carotid arteries 14 days after injury and treatment. Balloon damaged rat carotid arteries without MK2i-DCB treatment were stained with Masson's trichrome 14 days after injury. Red line indicates division between medial and neointimal layers.

FIG. 51C shows Gomori trichrome staining of drug coated balloon treated rat carotid arteries 14 days after injury and treatment. Balloon damaged rat carotid arteries with MK2i-DCB treatment were stained with Masson's trichrome 14 days after injury. Red line indicates division between medial and neointimal layers. Trichrome staining shows a denser collagen matrix in the neointima in the balloon damaged, untreated artery compared to the MK2i-DCB treated carotid.

FIG. 52A shows confocal fluorescence microscopy of undamaged carotid arteries stained for P-CREB.

FIG. 52B shows confocal fluorescence microscopy of untreated carotid arteries stained for P-CREB 14 d after injury.

FIG. 52C shows confocal fluorescence microscopy of drug coated balloon treated carotid arteries stained for P-CREB 14 d after injury to measure activity of MK2. DCB treatment inhibited P-CREB activation relative to untreated, balloon damaged samples.

FIG. 53 shows quantification of P-CREB in rat carotid arteries 14 days after balloon injury.

FIG. 54 shows a scanning electron microscopy (SEM) image of an example balloon coating. Dashed line indicates the coating thickness. White line indicates the balloon wall cross-section.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed technology. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are contemplated, and for the range 1.5-2, the numbers 1.5, 1.6, 1.7, 1.8, 1.9, and 2 are contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl,” as used herein, refers to a straight or branched, saturated hydrocarbon chain containing from 1 to 30 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and n-dodecyl. The alkyl group may be substituted or unsubstituted.

The term “effective dosage” or “therapeutic dosage” or “therapeutically effective amount” or “effective amount,” as used herein, refers to an amount sufficient to effect beneficial or desirable biological and/or clinical results, to modulate a biological process, and/or treat a disease or one or more of its symptoms and/or to prevent or reduce the risk of the occurrence or reoccurrence of the disease or disorder or symptom(s) thereof. A therapeutically effective amount is also one in which any toxic or detrimental effects of substance are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. In reference to treating a vascular disease, an effective or therapeutically effective amount can include an amount sufficient to, among other things, reduce the risk of restenosis.

“Subject” and “patient” as used herein interchangeably refers to any vertebrate, including, but not limited to, a mammal that wants or is in need of the herein described devices or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

“Substantially identical” means that a first and second amino acid sequence are at least 60%, 65%, 70%, 75%, 80%, 85%, 90%, 95%, 96%, 97%, 98%, or 99% identical over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, or 1100 amino acids. This can also be referred to as X % sequence identity, where a first and second amino acid sequence are at least X % identical over a region of amino acids as listed above.

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

The term “treatment” or “treating,” as used herein, refers to protection of a subject from a disease, such as preventing, suppressing, repressing, ameliorating, or eliminating the disease. Preventing the disease involves administering a device of the present disclosure to a subject prior to onset of the disease. Suppressing the disease involves administering a device of the present disclosure to a subject after induction of the disease but before its clinical appearance. Repressing or ameliorating the disease involves administering a device of the present disclosure to a subject after clinical appearance of the disease.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### 2. Drug Coated Medical Devices

Provided herein are drug coated medical devices. The drug coated medical device includes a medical device and a multi-layer drug coating on a surface of the medical device. The term medical device, as used herein, refers to an instrument, apparatus, implement, machine, implant, in vitro reagent, or other similar or related article which is intended to prevent, diagnose, and/or treat a disease or disorder, or intended to affect the structure and/or function of a body of a subject. The medical device can be any medical device intended to interact with a biological tissue. For example, the medical device can be one intended to interact with a subject's vasculature. Example medical devices include, but are not limited to, an angioplasty balloon catheter (cardiac or peripheral vascular angioplasty or for angioplasty of arteriovenous fistula or grafts created for dialysis access), other types of balloon catheters, a stent, a vascular graft (e.g., synthetic graft, vein graft, or a combination thereof), surgical tools, wound dressings, syringes, pacemakers, prosthetics, and the like. In some embodiments, the medical device comprises an angioplasty balloon catheter, a stent, or a vascular graft. In some embodiments, the medical device is an angioplasty balloon catheter, a stent, or a vascular graft.

The medical device can have an outer surface. The outer surface can have a coating applied thereto. For example, a multi-layer drug coating can be positioned on the outer surface of the medical device. The outer surface of the medical device can be capable of electrostatically interacting or binding with the multi-layer drug coating. The multi-layer drug coating can be positioned on at least a portion of the outer surface. In some embodiments, the multi-layer drug coating is positioned on substantially all of the outer surface. In some embodiments, the multi-layer drug coating is positioned on the entirety of the outer surface.

The medical device can also include a base layer between the outer surface and the multi-layer drug coating. The base layer can aid the interaction between the multi-layer drug coating and the medical device. The base layer can include a silane. The silane can be positively or negatively charged. In some embodiments, the silane is an aminosilane that can be positively charged. The base layer can be capable of electrostatically binding with the multi-layer drug coating.

In some embodiments, the medical device is an angioplasty balloon catheter including a catheter and an inflatable balloon coupled to the catheter. The inflatable balloon can have an outer surface, which can be the outer surface of the medical device.

### A. Multi-Layer Drug Coatings

The drug coating takes advantage of electrostatic interactions between different layers to form a multi-layer structure on the medical device. For example, the multi-layer drug coating can include at least one polyelectrolyte bilayer. As used herein, “polyelectrolyte bilayer” refers to a bilayer including a polyanionic layer and a polycationic layer, where the polyanionic layer and the polycationic layer are capable of electrostatically binding to each other. Accordingly, the polyelectrolyte bilayer includes a polycationic layer and a polyanionic layer.

The multi-layer drug coating can include one or more polyelectrolyte bilayers. For example, the multi-layer drug coating can include 1 to 20 polyelectrolyte bilayers, such as 1 to 18 polyelectrolyte bilayers, 1 to 15 polyelectrolyte bilayers, 1 to 10 polyelectrolyte bilayers, 1 to 8 polyelectrolyte bilayers, 1 to 6 polyelectrolyte bilayers, 2 to 12 polyelectrolyte bilayers, 2 to 10 polyelectrolyte bilayers, 2 to 8 polyelectrolyte bilayers, 2 to 6 polyelectrolyte bilayers, 3 to 12 polyelectrolyte bilayers, 3 to 10 polyelectrolyte bilayers, 3 to 8 polyelectrolyte bilayers, 3 to 6 polyelectrolyte bilayers, 4 to 10 polyelectrolyte bilayers, 4 to 8 polyelectrolyte bilayers, or 4 to 6 polyelectrolyte bilayers.

In some embodiments, the multi-layer drug coating includes less than 20 polyelectrolyte bilayers, less than 19 polyelectrolyte bilayers, less than 18 polyelectrolyte bilayers, less than 17 polyelectrolyte bilayers, less than 16 polyelectrolyte bilayers, less than 15 polyelectrolyte bilayers, less than 14 polyelectrolyte bilayers, less than 13 polyelectrolyte bilayers, less than 12 polyelectrolyte bilayers, less than 11 polyelectrolyte bilayers, or less than 10 polyelectrolyte bilayers. In some embodiments, the multi-layer drug coating includes greater than 1 polyelectrolyte bilayer, greater than 2 polyelectrolyte bilayers, greater than 3 polyelectrolyte bilayers, greater than 4 polyelectrolyte bilayers, greater than 5 polyelectrolyte bilayers, greater than 6 polyelectrolyte bilayers, greater than 7 polyelectrolyte bilayers, greater than 8 polyelectrolyte bilayers, greater than 9 polyelectrolyte bilayers, or greater than 10 polyelectrolyte bilayers. In some embodiments, the multi-layer drug coating includes a plurality of polyelectrolyte bilayers.

The multi-layer drug coating can have corresponding numbers of polycationic layers and polyanionic layers (e.g., 4 polycationic layers and 4 polyanionic layers). However, in some embodiments, the multi-layer drug coating has more polycationic layers compared to polyanionic layers, or vice versa (e.g., 5 polycationic layers and 4 polyanionic layers).

The multi-layer drug coating can have a varying thickness. For example, the multi-layer drug coating can have a thickness of about 0.5 μm to about 4 μm, such as about 0.75 μm to about 3.5 μm, about 1 μm to about 3 μm, about 0.5 μm to about 2.5 μm, about 1 μm to about 2.5 μm, about 1 μm to about 2 μm, or about 2 μm to about 4 μm. In some embodiments, the multi-layer drug coating has a thickness of less than 4 μm, less than 3.5 μm, less than 3 μm, less than 2.5 μm, less than 2 μm, less than 1.5 μm, or less 1 μm. In some embodiments, the multi-layer drug coating has a thickness of greater than 0.5 μm, greater than 0.75 μm, greater than 1 μm, greater than 1.25 μm, greater than 1.5 μm, greater than 1.75 μm, greater than 2 μm, or greater 2.5 μm. The thickness of the multi-layer drug coating can be measured by techniques known within the art such as microscopy (e.g., fluorescence microscopy, electron microscopy, etc.).

i. Polycationic Layer

The polycationic layer includes a cationic therapeutic. As used herein, a “cationic therapeutic” refers to a therapeutic that has a net positive charge at a pH of about 6 to about 8, such that it can electrostatically interact with the anionic polymer. Accordingly, the cationic therapeutic can instill cationic properties to the polycationic layer (e.g., a positive surface charge). The charge of the polycationic layer can be measured by atomic force microscopy (AFM). Example cationic therapeutics include, but are not limited to, cationic peptides, cationic oligonucleotide analogs (e.g., with cationic backbone linkages), vivo-morpholinos (e.g., antisense (morpholino) conjugated to a cationic dendrimer), peptides conjugated or fused with cationic peptides or cationic dendrimers, cationic nanoparticles, cationic conjugates (e.g., conjugate combinations of polymers, peptides, polynucleotides, and/or small molecule drugs). In some embodiments, the cationic therapeutic includes a cationic peptide, a cationic oligonucleotide analog, a cationic conjugate, a cationic nanoparticle, or a combination thereof. In some embodiments, the cationic therapeutic includes a cationic peptide or a cationic nanoparticle.

The cationic therapeutic can be a cationic peptide. As used herein, a “cationic peptide” refers to a peptide that has a net positive charge at a pH of about 6 to about 8, such that it can electrostatically interact with the anionic polymer. Example cationic peptides include, but are not limited to, p38 pathway inhibitory peptides (e.g., MAPKAP Kinase II inhibitory peptide (MK2i)), cell penetrating peptides (e.g., R6, TAT, penetratin, transportan, etc.), and fusions thereof. In some embodiments, the cationic peptide includes MK2i, a cell penetrating peptide, or a combination thereof. Combinations can include fusions thereof or combinations of individual, distinct cationic peptides.

In some embodiments, the cationic peptide includes an amino acid sequence having at least 80%, 85%, 90%, 95%, 99%, or about 100% identity to SEQ ID NO:1, SEQ ID NO:2, SEQ ID NO:3, SEQ ID NO:4, or SEQ ID NO:5. In some embodiments, the cationic peptide includes an amino acid sequence selected from the group consisting of SEQ ID NO:1, SEQ ID NO: 2, SEQ ID NO:3, SEQ ID NO:4, and SEQ ID NO:5. In some embodiments, the cationic peptide includes an amino acid sequence of SEQ ID NO:1.

The multi-layer drug coating can include the cationic therapeutic at varying amounts. For example, the multi-layer drug coating can include the cationic therapeutic at about 0.5 μg/cm² to about 10 μg/cm², such as about 0.75 μg/cm² to about 8 μg/cm², about 1 μg/cm² to about 6 μg/cm², about 3 μg/cm² to about 6 μg/cm², about 2 μg/cm² to about 10 μg/cm², or about 1 μg/cm² to about 7 μg/cm². In some embodiments, the multi-layer drug coating includes the cationic therapeutic at less than 10 μg/cm², less than 9 μg/cm², less than 8 μg/cm², less than 7 μg/cm², less than 6 μg/cm², or less than 5 μg/cm². In some embodiments, the multi-layer drug coating includes the cationic therapeutic at greater than 0.5 μg/cm², greater than 0.75 μg/cm², greater than 1 μg/cm², greater than 1.5 μg/cm², greater than 2 μg/cm², greater than 2.5 μg/cm², or greater than 3 μg/cm². The foregoing amounts of the cationic therapeutic can also be applied to the cationic peptide.

The multi-layer drug coating can include about 1 μg to about 12 μg of cationic therapeutic, such as about 1 μg to about 11 μg of cationic therapeutic, about 1.5 μg to about 10 μg of cationic therapeutic, about 2 μg to about 9 μg of cationic therapeutic, about 1 μg to about 6 μg of cationic therapeutic, about 4 μg to about 12 μg of cationic therapeutic, about 5 μg to about 10 μg of cationic therapeutic, or about 6 μg to about 9 μg of cationic therapeutic. In some embodiments, the multi-layer drug coating includes less than 12 μg of cationic therapeutic, less than 11 μg of cationic therapeutic, less than 10 μg of cationic therapeutic, less than 9 μg of cationic therapeutic, or less than 8 μg of cationic therapeutic. In some embodiments, the multi-layer drug coating includes greater than 1 μg of cationic therapeutic, greater than 1.5 μg of cationic therapeutic, greater than 2 μg of cationic therapeutic, greater than 2.5 μg of cationic therapeutic, or greater than 3 μg of cationic therapeutic. The foregoing amounts of the cationic therapeutic can also be applied to the cationic peptide.

The multi-layer drug coating can include one or more polycationic layers. For example, the multi-layer drug coating can include 1 to 20 polycationic layers, such as 1 to 15 polycationic layers, 1 to 12 polycationic layers, 1 to 10 polycationic layers, 2 to 14 polycationic layers, 2 to 10 polycationic layers, 1 to 8 polycationic layers, 2 to 8 polycationic layers, 1 to 6 polycationic layers, 2 to 6 polycationic layers, 1 to 5 polycationic layers, 1 to 4 polycationic layers, 1 to 3 polycationic layers, or 3 to 7 polycationic layers. In some embodiments, the multi-layer drug coating includes less than 20 polycationic layers, less than 15 polycationic layers, less than 12 polycationic layers, less than 10 polycationic layers, less than 8 polycationic layers, less than 7 polycationic layers, or less than 6 polycationic layers. In some embodiments, the multi-layer drug coating includes greater than 1 polycationic layer, greater than 2 polycationic layers, greater than 3 polycationic layers, greater than 4 polycationic layers, or greater than 5 polycationic layers. In some embodiments, the multi-layer drug coating includes a plurality of polycationic layers.

ii. Polyanionic Layer

The polyanionic layer includes an anionic polymer. As used herein, an “anionic polymer” refers to a polymer that has a net negative charge at a pH of about 6 to about 8, such that it can electrostatically interact with the cationic therapeutic (e.g., cationic peptide). Accordingly, the anionic polymer can instill anionic properties to the polyanionic layer (e.g., a negative surface charge). Similar to the polycationic layer, the charge of the polyanionic layer can be measured by AFM.

The anionic polymer can include recurring units of formula (I)

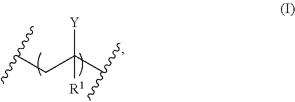

wherein Y is —C(O)OH, —SO₃H, or —PO₃H₂, and R¹ is C₁-C₁₂ alkyl.

In some embodiments, Y is —C(O)OH or —SO₃H. In some embodiments, Y is —C(O)OH.

In some embodiments, R¹ is C₁-C₁₀ alkyl. In some embodiments, R¹ is C₂-C₁₀ alkyl. In some embodiments, R¹ is C₃-C₁₀ alkyl. In some embodiments, R¹ is C₁-C₈ alkyl. In some embodiments, R¹ is C₁-C₆ alkyl. In some embodiments, R¹ is C₁-C₅ alkyl. In some embodiments, R¹ is C₂-C₄ alkyl. R¹ can be optionally substituted.

In some embodiments, the recurring units of formula (I) can be of formula (I-a)

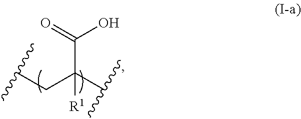

wherein R¹ is C₁-C₆ alkyl.

In some embodiments, the anionic polymer has recurring units of formula (I-a), wherein R¹ is C₁-C₅ alkyl. In some embodiments, the anionic polymer has recurring units of formula (I-a), wherein R¹ is C₂-C₄ alkyl.

The anionic polymer can have a varying pKa, which can be used to control the pH responsiveness of the anionic polymer. For example, the anionic polymer can have a pKa of about 6 to about 7.4, such as about 6 to about 7.2, about 6.1 to about 7.1, about 6.2 to about 7, about 6.4 to about 7, about 6.5 to about 7.1, about 6.6 to about 6.8, about 6 to about 6.5, or about 6.5 to about 7.2. In some embodiments, the anionic polymer has a pKa of less than 7.4, less than 7.2, less than 7.1, less than 7, less than 6.8, less than 6.7, less than 6.6, or less than 6.5. In some embodiments, the anionic polymer has a pKa of greater than 6, greater than 6.1, greater than 6.2, greater than 6.3, greater than 6.4, greater than 6.5, or greater than 6.6. In some embodiments, the anionic polymer has a pKa of about 6.7. The pKa of the anionic polymer can be determined through techniques known within the art including, but not limited to, potentiometry, spectrometry, voltammetry, electrophoresis, nuclear magnetic resonance (NMR), and molecular simulation.

The anionic polymer can have advantageous properties that can aid in intracellular delivery of the cationic therapeutic. For example, the anionic polymer can be pH responsive. As shown in formula (I), the anionic polymer can include protonatable functional groups (e.g., Y) and alkyl groups (e.g., R¹). The protonatable functional groups can instill an anionic character to the anionic polymer when the pH is above the anionic polymer's pKa. However, when the pH is decreased (e.g., to or below the pKa of the anionic polymer) and the protonatable functional groups increase in protonation, the anionic character of the anionic polymer can be decreased. Upon a decrease in anionic character of the anionic polymer, the anionic polymer can transition between a water-soluble structure to a collapsed structure, which can express the hydrophobic properties (e.g., the alkyl side chain) of the anionic polymer. This pH responsive property can aid in the anionic polymer in destabilizing biological membranes through the alkyl side chain, which can in turn facilitate endosome disruption and cytoplasmic delivery of, e.g., the cationic therapeutic. In some embodiments, the alkyl side chain facilitates membrane interactions that aid cell uptake, endosome escape, or both. In some embodiments, the pKa and the alkyl side chain of the anionic polymer combine to facilitate endosome disruption and cytoplasmic delivery of the cationic therapeutic.

The anionic polymer can be synthesized via polymerization techniques known within the art, such as reversible addition-fragmentation chain-transfer (RAFT) polymerization. The anionic polymer can be a homopolymer or a copolymer. For example, the anionic polymer can be a homopolymer including recurring units derived from the same monomer. In contrast, the anionic polymer can be a copolymer including recurring units that are derived from two or more different monomers. Different recurring units of a copolymer can have different R¹ groups (e.g., C₃ alkyl and C₅ alkyl), different Y groups (e.g., —C(O)OH and —SO₃H), or a combination thereof. The different recurring units of a copolymer can also be included at different molar ratios and in different copolymer structures (e.g., random, block, etc.). In addition, the anionic polymer can include other recurring units (other than those of formula (I) and (I-a)) derived from other monomers that can be used in, e.g., RAFT polymerization. Example polymerizations can be found in the Examples herein.

The anionic polymer can have a varying number average molecular weight. For example, the anionic polymer can have a number average molecular weight of about 10 kilodaltons (kDa) to about 40 kDa, such as about 15 kDa to about 35 kDa, about 20 kDa to about 30 kDa, about 20 kDa to about 25 kDa, about 10 kDa to about 25 kDa, about 20 kDa to about 40 kDa, or about 15 kDa to about 30 kDa. In some embodiments, the anionic polymer has a number average molecular weight of less than 40 kDa, less than 35 kDa, less than 30 kDa, less than 25 kDa, or less than 20 kDa. In some embodiments, the anionic polymer has a number average molecular weight of greater than 10 kDa, greater than 15 kDa, greater than 20 kDa, or greater than 25 kDa.

The molecular weight of the anionic polymer can be increased or decreased by altering polymerization conditions, such as monomer input. In addition, molecular weight of the anionic polymer can be measured by techniques known within the art, such as size exclusion chromatography (SEC), SEC combined with multi-angle light scattering, gel permeation chromatography (GPC), rheometry, and the like. For example, the above listed molecular weights of the anionic polymer can be measured by GPC.

The multi-layer drug coating can include one or more polyanionic layers. For example, the multi-layer drug coating can include 1 to 20 polyanionic layers, such as 1 to 15 polyanionic layers, 1 to 12 polyanionic layers, 1 to 10 polyanionic layers, 2 to 14 polyanionic layers, 2 to 10 polyanionic layers, 1 to 8 polyanionic layers, 2 to 8 polyanionic layers, 1 to 6 polyanionic layers, 2 to 6 polyanionic layers, 1 to 5 polyanionic layers, 1 to 4 polyanionic layers, 1 to 3 polyanionic layers, or 3 to 7 polyanionic layers. In some embodiments, the multi-layer drug coating includes less than 20 polyanionic layers, less than 15 polyanionic layers, less than 12 polyanionic layers, less than 10 polyanionic layers, less than 8 polyanionic layers, less than 7 polyanionic layers, or less than 6 polyanionic layers. In some embodiments, the multi-layer drug coating includes greater than 1 polyanionic layer, greater than 2 polyanionic layers, greater than 3 polyanionic layers, greater than 4 polyanionic layers, or greater than 5 polyanionic layers. In some embodiments, the multi-layer drug coating includes a plurality of polyanionic layers.

iii. Excipients

The multi-layer drug coating can also include an excipient, such as a saccharide. It has been found that the presence of the saccharide can improve the release of the different layers from the surface of the medical device, and thus can improve the release of the cationic therapeutic from the medical device. The saccharide can be a monosaccharide, a disaccharide, an oligosaccharide, a polysaccharide, or a combination thereof. In some embodiments, the saccharide includes a monosaccharide, a disaccharide, a trisaccharide, or a combination thereof. In some embodiments, the saccharide includes a monosaccharide, a disaccharide, or a trisaccharide. In some embodiments, the saccharide includes a trisaccharide. Example saccharides include, but are not limited to, lactosucrose, trehalose, and sucrose. In some embodiments, the saccharide includes lactosucrose.

The saccharide can be included in the multi-layer drug coating in various locations. For example, the saccharide can be included in the polycationic layer, the polyanionic layer, or both. In some embodiments, the saccharide is included in the polycationic layer and the polyanionic layer. In addition, in embodiments with a plurality of polyelectrolyte bilayers, the saccharide can be included in all of the polyelectrolyte bilayers or in a portion of the polyelectrolyte bilayers.

Other example excipients include, but are not limited to, urea, polysorbate/sorbitol, shellac, iopromide, polyethylene glycol, and dextran. Further description of excipients can be found in Huiying et al., Drug-Coated Balloons: Technologies and Clinical Applications, Current Pharmaceutical Design 24 (4), December 2017, which is incorporated by reference herein in its entirety.

### 3. Methods of Making the Drug Coated Medical Devices

Further disclosed herein are methods of making the drug coated medical devices. The multi-layer drug coatings can be assembled on the medical device using layer-by-layer techniques. Layer-by-layer techniques can be used to coat a wide variety of substrates using charged materials via electrostatic self-assembly. In the layer-by-layer technique, a first layer having a first surface charge is typically deposited on an underlying substrate (e.g., the medical device), followed by a second layer having a second surface charge that is opposite in sign to the surface charge of the first layer, and so forth. The charge on the outer layer is reversed upon deposition of each sequential layer. Layer-by-layer coatings can be applied by a number of techniques known in the art such as, but not limited to, immersion and spraying techniques.

The method can include immersing an outer surface of a medical device into a first mixture, the first mixture comprising a first solvent and an anionic polymer having recurring units of formula (I) as described herein to provide a polyanionic layer on the outer surface of the medical device, wherein the polyanionic layer includes the anionic polymer. In some embodiments, the outer surface of the medical device can be dried and immersed again in the first mixture prior to immersing in the second mixture.

The anionic polymer can be included in the first mixture at varying concentrations. For example, the first mixture can include the anionic polymer at about 5 μM to about 10 μM, such as about 6 μM to about 9 μM, about 5 μM to about 8 μM, or about 7 μM to about 10 μM. In some embodiments, the first mixture includes the anionic polymer at less than 10 μM, less than 9 μM, or less than 8 μM. In some embodiments, the first mixture includes the anionic polymer at greater than 5 μM, greater than 6 μM, or greater than 7 μM.

The method can further include immersing the outer surface of the medical device into a second mixture, the second mixture comprising a second solvent and a cationic therapeutic, to provide a polycationic layer on a surface of the polyanionic layer, wherein the polycationic layer includes the cationic therapeutic. In some embodiments, the outer surface of the medical device can be dried and immersed again in the second mixture prior to any subsequent immersing in the first mixture.

The method can also include the immersion steps in reverse order. For example, the first immersion step can be done with the second mixture followed by an immersion step with the first mixture as describe herein.

The cationic therapeutic can be included in the second mixture at varying concentrations. For example, the second mixture can include the cationic therapeutic at about 50 μM to about 100 μM, such as about 60 μM to about 90 μM, about 65 μM to about 85 μM, about 80 μM to about 100 μM, or about 50 μM to about 70 μM. In some embodiments, the second mixture includes the cationic therapeutic at less than 100 μM, less than 95 μM, or less than 90 μM. In some embodiments, the second mixture includes the cationic therapeutic at greater than 50 μM, greater than 55 μM, or greater than 60 μM. The foregoing concentrations of the cationic therapeutic can also be applied to the cationic peptide.

The outer surface of the medical device can be capable of electrostatically binding to the anionic polymer or the cationic therapeutic. The outer surface of the medical device may be surface treated prior to immersing in the first mixture. For example, the outer surface of the medical device may be plasma treated, silane treated, or both prior to immersing in the first mixture. In some embodiments, the outer surface of the medical device is plasma treated and silane treated prior to immersing in the first mixture. In some embodiments, the silane is an aminosilane.

The first solvent and the second solvent can be any suitable solvent that can dissolve the anionic polymer and cationic therapeutic, respectively—as well as the saccharide if present. In some embodiments, the first solvent and the second solvent are the same. In some embodiments, the first solvent and the second solvent are different. In some embodiments, the first solvent and the second solvent are each independently an alcohol. In some embodiments, the first solvent and the second solvent are each independently methanol.

The method can include optionally repeating the two immersion steps (e.g., immersing in the first mixture+immersing in the second mixture). These steps can be repeated any number of times, where a round of the two immersion steps can provide a polyelectrolyte bilayer on the medical device.

The method can further include a saccharide as described herein present in the first mixture, the second mixture, or both. In some embodiments, the saccharide is included in both the first mixture and the second mixture. In some embodiments, the saccharide includes lactosucrose, trehalose, or sucrose.

The saccharide can be included in the first mixture, the second mixture, or both in varying concentrations. For example, the first mixture, the second mixture or both can independently include the saccharide at about 50 mM to about 150 mM, such as about 75 mM to about 125 mM, about 80 mM to about 110 mM, about 50 mM to about 100 mM, or about 100 mM to about 150 mM. In some embodiments, the first mixture, the second mixture, or both independently include the saccharide at less than 150 mM, less than 125 mM, or less than 100 mM. In some embodiments, first mixture, the second mixture, or both independently include the saccharide at greater than 50 mM, greater than 75 mM, or greater than 100 mM. In some embodiments, the first mixture, the second mixture, or both independently include the saccharide at about 100 mM.

The description of the medical devices, multi-layer drug coatings, polyelectrolyte bilayers, saccharides, polycationic layers, and polyanionic layers above can also be applied to the methods of making drug coated medical devices disclosed herein.

### 4. Methods of Using the Drug Coated Medical Devices

Disclosed herein are methods of treating a vascular disease in a subject (e.g., in need thereof). The method can include implanting the medical device as disclosed herein at a vascular site of the subject. Example vascular sites include, but are not limited to, a blood vessel, a vein graft, a synthetic graft, or an arteriovenous fistula. In some embodiments, the vascular site is a blood vessel or an arteriovenous fistula.

The method can reduce the risk of restenosis in the subject. Accordingly, the vascular disease can be any vascular disease that can benefit from deceasing the incidence of restenosis. Example vascular diseases include, but are not limited to, intimal hyperplasia (IH), peripheral artery disease, atherosclerosis, arteriovenous fistula stenosis, coronary artery disease, and combinations thereof. In some embodiments, the vascular disease is peripheral artery disease.

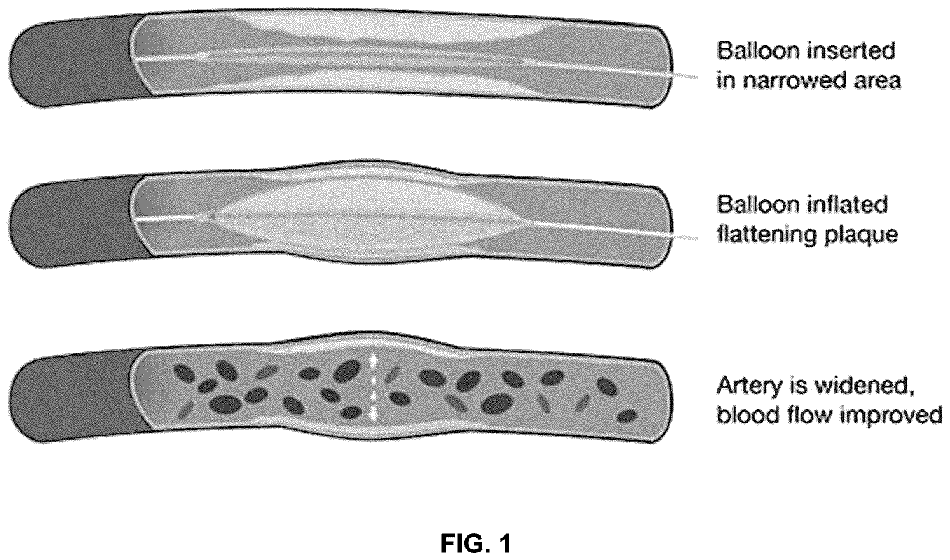

Also disclosed are methods of delivering a cationic therapeutic to a subject (e.g., in need thereof). The method can include (a) implanting a drug coated angioplasty balloon catheter as disclosed herein at a vascular site of the subject; (b) inflating the balloon of the medical device, thereby transferring a portion of the cationic therapeutic from the medical device to the vascular site; (c) deflating the balloon of the medical device; (d) optionally repeating steps (b) and (c); and (e) removing the medical device from the vascular site. An example angioplasty balloon catheter application can be seen in FIG. 1.

The cationic therapeutic of the multi-layer drug coating can be advantageously transferred from the medical device to surrounding tissue by controlling the stability of the multi-layer drug coating as described herein. In some embodiments, at least 0.5% by weight, at least 1% by weight, at least 1.5% by weight, at least 2% by weight, at least 2.5% by weight, at least 3% by weight, at least 4% by weight, at least 5% by weight, or at least 10% by weight of the cationic therapeutic is transferred from the balloon to the vascular site following removal of the medical device. The foregoing weight percentages of transferred cationic therapeutic can also be applied to the cationic peptide.

The description of the medical devices, multi-layer drug coatings, polyelectrolyte bilayers, saccharides, polycationic layers, and polyanionic layers above can also be applied to the methods of treating and delivering disclosed herein.

The disclosed technology has multiple aspects, illustrated by the following non-limiting examples.

### 5. Examples

### Materials & Methods for Examples 1 and 2

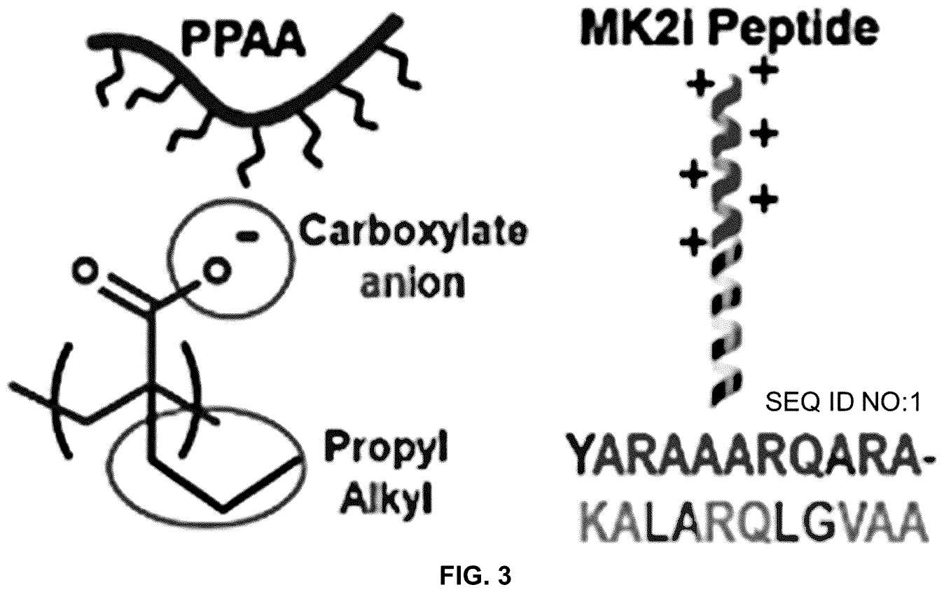

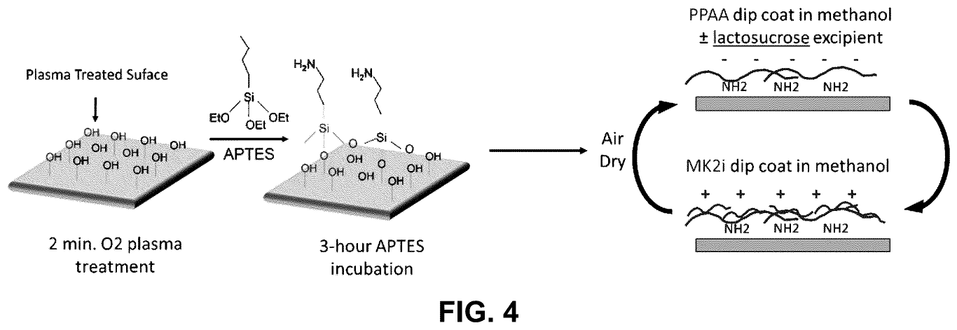

Layer-by-layer balloon coating: Angioplasty balloons were obtained from Medical Materials, Inc. To establish a positively charged base layer, the balloon was plasma treated for 2 minutes to oxidize the surface and then immediately placed into a 1 w/w % solution of APTES in 50% ethanol for 3 hours (FIG. 4—see, e.g., Zhang et al., Maskless Surface Modification of Polyurethane Films by an Atmospheric Pressure He/O₂ Plasma Microjet for Gelatin Immobilization, Micromachines, 2018, 9, 195, which is incorporated by reference herein in its entirety). To coat the balloon, solutions of 50 μM MK2i and 5 μM PPAA (FIG. 3) were made in methanol (these concentrations were increased to 100 μM and 10 μM, respectively). The balloon was then dipped in the PPAA solution briefly and allowed to air dry for one minute. This step was repeated to establish a thorough coating of PPAA on the balloon. This process was then repeated with the MK2i solution to create a layer on top of the PPAA layer. These steps were repeated to create 6 PPAA/MK2i bilayers on the balloon. Balloon layering was confirmed by using Alexa-568 labeled MK2i and visualizing the coating on IVIS.

Balloon loading and release quantification: Angioplasty balloons (2.5 mm×20 mm) were coated with PPAA and MK2i-568 and then soaked in PBS with calcium and magnesium for 3 minutes to measure balloon release. The balloons were then soaked again in DMSO under sonication for 3 minutes to remove any remaining MK2i layers. The fluorescence of the PBS and DMSO solutions was measured on a TECAN Infinite M1000 Pro plate reader and compared to a standard curve of MK2i-568 in PBS and DMSO, respectively.

Rat aorta harvest: Retired breeder Sprague-Dawley rats were ordered from Charles River Laboratories. Rats were euthanized with CO₂ before harvest of abdominal aortas. The chest cavity was opened and fascia and other tissue surrounding the aorta was carefully removed. Branches from the aorta were cauterized with a Bovie cautery pen to allow for vessel pressurization. The aorta was harvested from the branch to the abdominal cavity and was placed in DMEM (+HEPES, +L-glutamine, +3% FBS, +1% Antibiotic/Antimycotic) until ready for experimentation.

Aorta treatment: Harvested aortas were damaged with an angioplasty balloon prior to treatment (3×, 15 seconds each, 25% overstretch). Coated balloons were then inserted into the aorta and pressurized for 3 minutes in DMEM (+HEPES, +L-glutamine, +3% FBS, +1% Antibiotic/Antimycotic), with deflation and reinflation at each minute mark to allow for maximum release and tissue transfer. After 3 minutes, the balloon was deflated and removed. The treated artery was then washed briefly with DMEM to remove excess MK2i and then processed for further experimentation or data collection. For convective delivery, after initial angioplasty damage, the aortas were connected to a pressure sensor and filled with treatment solution (50 HM MK2i, 50 μM MK2i-NPs, or 5 μM PPAA then 50 μM MK2i for PPAA->MK2i). The open end of the aorta was then clamped off and a syringe was used to pressurize the treatment solution within the artery to 150 mmHg. This pressure was held for 3 minutes (for PPAA->MK2i, each step was held for 3 minutes). The treated aorta was then disconnected from the clamp and pressure sensor, washed briefly to remove any excess MK2i, and then processed for further experimentation or data collection.

Delivery visualization and quantification: Immediately after aorta treatment, samples were placed into OCT and frozen for cryosectioning. Samples were cut into 10 μm sections and secured onto slides using ProLong Gold Antifade mounting media with DAPI. After drying, the slides were scanned on a Nikon Eclipse Ti confocal microscope to measure the intensity of the fluorescent MK2i within the arterial cross-section. An ROI was drawn around the outside and inside edges of the vessel using automatic ROI detection on the DAPI channel. Then, total intensity of MK2i-568 within the arterial wall was calculated by subtracting the intensity of the inside segment from the intensity of the total segment. The same was done to calculate the area of the arterial wall and then the intensity was normalized to the area for each sample. Additionally, a delivery profile was created by drawing a line (lumen to adventitia) at 8 evenly spaced points around the arterial wall and averaging the intensity along those lines. This created a measurement of MK2i delivery as a function of distance into the arterial wall.

Flow Cytometry: After aorta treatment, samples were incubated in DMEM (+HEPES, +L-glutamine, +3% FBS, +1% Antibiotic/Antimycotic) for 1 hour to allow for cell uptake. The aortas were subsequently transferred to 500 μL of cold PBS+1% FBS and cut with scissors into small (˜1 mm) sized pieces. Digestion media in DMEM was then added to the PBS/tissue tube (500 μL) to achieve a final enzyme concentration of 0.7 mg/mL liberase, 4 mg/mL collagenase, and 0.2 mg/mL DNase and the tubes were placed in a 37° C. incubator on a shaker for ˜90 minutes or until digestion was apparent. After incubation, the samples were filtered through a 70 μm cell strainer and centrifuged at 500 g for 8 minutes. The resulting cell pellet was resuspended in 200 μL of FACS buffer (PBS+/+, 1% FBS, 2 mM EDTA) and transferred to a 96 well plate for flow cytometry. Cells were read for fluorescence on a Guava easyCyte HT and the resulting data was processed in FlowJo.

Flow Loop Bioreactor Setup: To prepare the flow loop system for aorta incubation, a sterile 4 mm biopsy punch was used to make holes in sterile 50 ml conical tubes. Connecting tubes were sterilized with ethanol and washed through with 3% hydrogen peroxide followed by DI water. Treated aortas were hung inside each 50 mL tube and stretched to 30% of their basal level to simulate physiological tension. The media used was based on methods previously described by Wang, et al., Biomaterials, 2021, which is incorporated by reference herein in its entirety. Flow loop media was created by mixing 50% DMEM and 50% VascuLife growth media supplemented with 1% Antibiotic/Antimycotic and 30 g/L dextran to increase the viscosity of the media to be similar to blood. Aortas were connected to tubing with plastic cannulas, secured with sterile sutures. The flow rate was set to 6 mL/min based on creating a shear stress similar to that of atherosclerotic arterial regions. Doriot's equation was used to calculate the ideal flow rate with a set viscosity of 0.043 dyn s/cm² and average arterial diameter of 1.8 mm based on previously obtained histological sections. The media flowing through the aorta was replaced every other day, while 30% of the media circulating around the aorta was replenished every day.

Western Blotting for pCREB inhibition: For initial 1 hr inhibition of pCREB, aorta samples were divided in half. One half underwent initial balloon damage and then a vehicle control (PBS for convective delivery, uncoated balloon for balloon delivery) for 3 minutes. The other half of each sample underwent MK2i treatment as normal. The samples were incubated for 1 hr after balloon damage/treatment in DMEM (+HEPES, +L-glutamine, +3% FBS, +1% Antibiotic/Antimycotic) before being processed for western blotting. For long term CREB inhibition, aorta samples were incubated in the flow loop for 24 h after treatment. Each aorta was then divided in half and transferred to flow loop media with or without 120 μM LPA for 2 hours. The samples were then flash frozen in liquid nitrogen. Frozen aortas were homogenized with a tissue pulverizer and lysed in RIPA buffer with protease and phosphatase inhibitors (Roche). The RIPA+tissue homogenate was vortexed for 15 minutes at 4° C. and then centrifuged at 12,000 g for 15 minutes at 4° C. to collect the supernatant. Protein concentration was quantified using a BCA Assay kit (Pierce). Protein (40 μg) was resolved on 4-10% SDS-PAGE gels and transferred to a nitrocellulose membrane using the Invitrogen iBlot 2. Blots were incubated in primary pCREB antibody (CST, 1:1000) overnight at 4° C. and incubated in the secondary antibody for 1 hr at room temperature. Blots were imaged on a LiCor Odyssey Fc.

MK2i Delivery and Retention Quantification: Tissue lysates prepped for western blotting were measured for presence of fluorescently labeled MK2i to quantify tissue delivery and retention. Tissue lysate fluorescence was measured on a TECAN Infinite M1000 Pro plate reader and compared to a standard curve of fluorescent MK2i to calculate tissue MK2i concentration.

Immunofluorescence for cell proliferation: Rat aortas were balloon damaged and either treated or left untreated before incubation in the flow loop bioreactor for 7 days. Aorta samples were then frozen in OCT and 5 μm sections were taken on slides. These slides were fixed and stained for Ki67 to measure cellular proliferation. Positively stained cells in the adventitial layer were manually counted for each sample and normalized to total cell count.

### Example 1

### Balloon Characterization

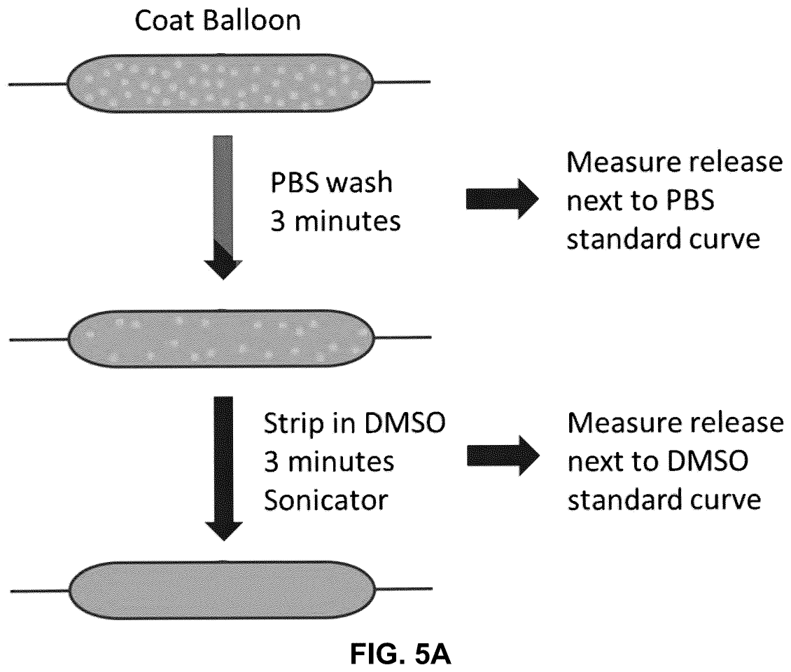

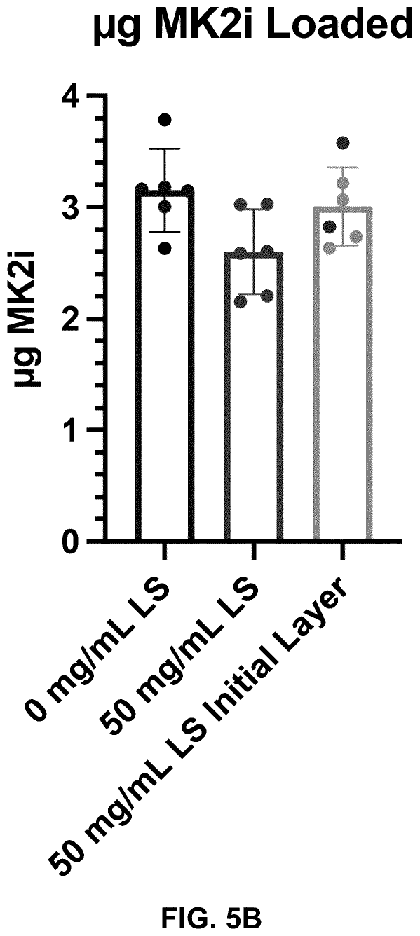

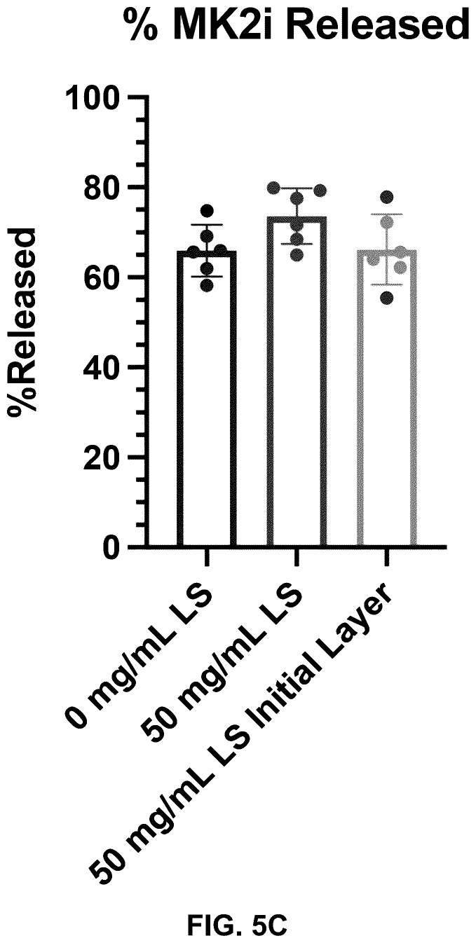

Balloon Loading and Release: Balloon release and total loading was quantified for different lactosucrose (LS) coating conditions. Balloons were coated with 6 layers of MK2i+PPAA (50 μM MK2i, 5 μM PPAA) without LS, with LS in the PPAA layers (50 mg/mL LS), or with LS only in the first PPAA layer. Coated balloons were soaked in PBS for 3 minutes to assess rapid release of MK2i+PPAA layers (FIG. 5A). The balloon coating was then stripped off in DMSO under sonication. The fluorescence of these two solutions was then measured and used to quantify MK2i concentration (FIG. 5B and FIG. 5C).

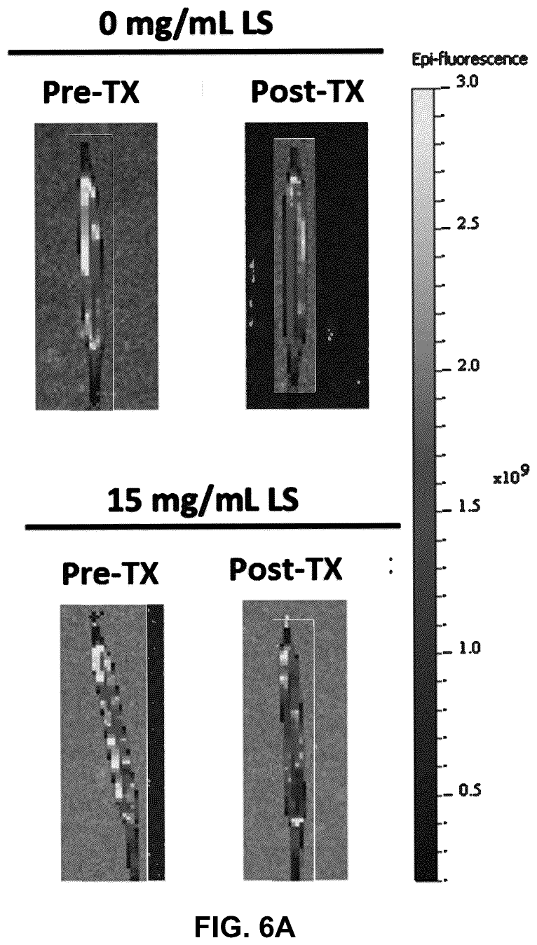

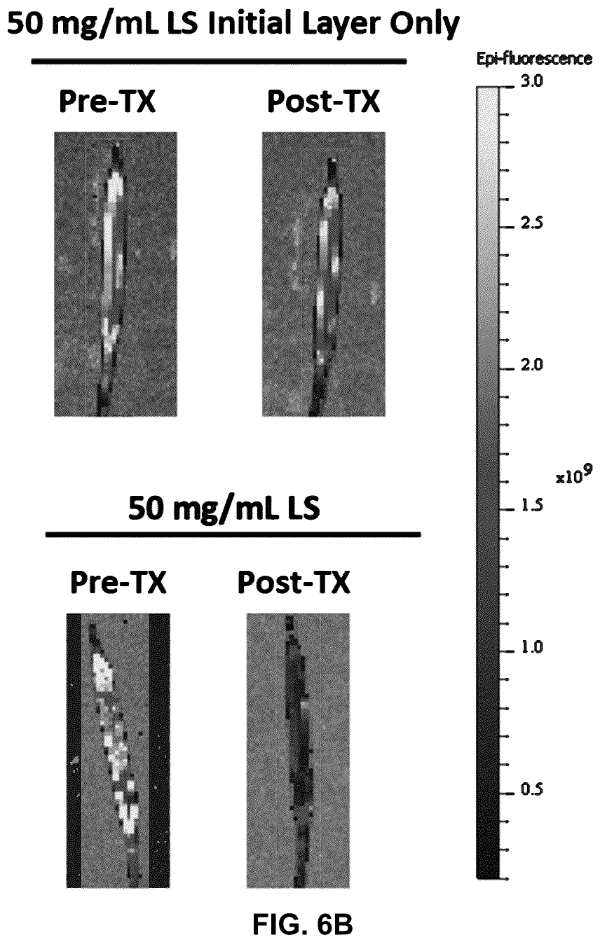

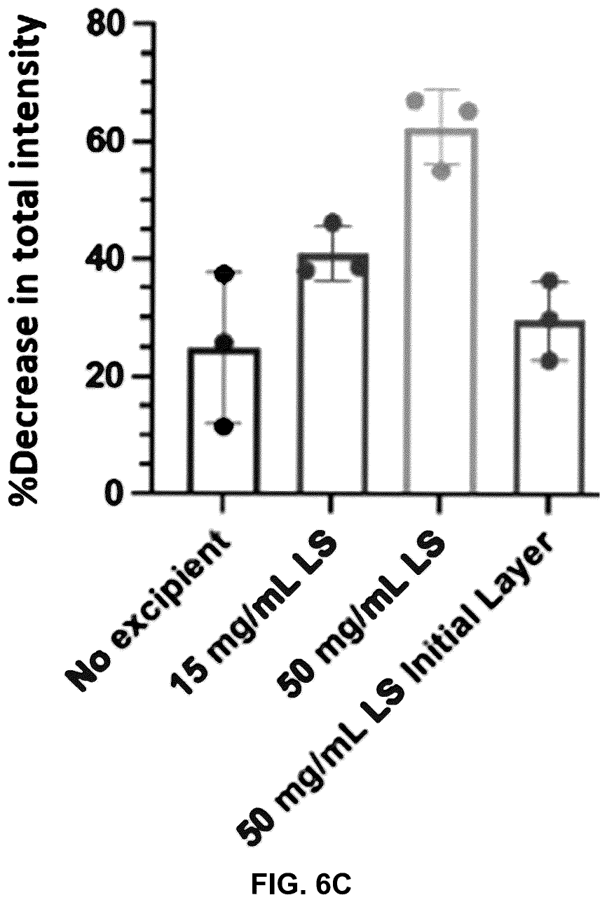

Effect of Lactosucrose: To show the effect of LS on the release of the MK2i+PPAA coating, coated balloons were inflated in ex vivo rat aortas for 3 minutes and the fluorescence before and after inflation was compared with IVIS imaging. ROIs were drawn around the balloons and the difference in fluorescence was calculated for the different coating formulations (FIG. 6A, FIG. 6B, and FIG. 6C).

Increasing balloon loading and release: To increase the amount of MK2i loaded onto and released from the balloons, two strategies were compared. Balloons were coated with either double the number of MK2i/PPAA layers (12 layers, 50 μM MK2i, 5 μM PPAA) or double the concentration of the coating solution (6 layers, 100 μM MK2i, 10 μM PPAA). These two conditions were additionally tested with and without the presence of 50 mg/mL LS in the PPAA layering. Loading and release were measured as before—3 min wash in PBS followed by sonication in DMSO. The solution fluorescence was measured to determine loading and release. Both methods of coating increased the amount of MK2i loaded, but doubling the layers resulted in lower MK2i released from the balloon than doubling the concentration.

### Example 2

### MK2i Delivery

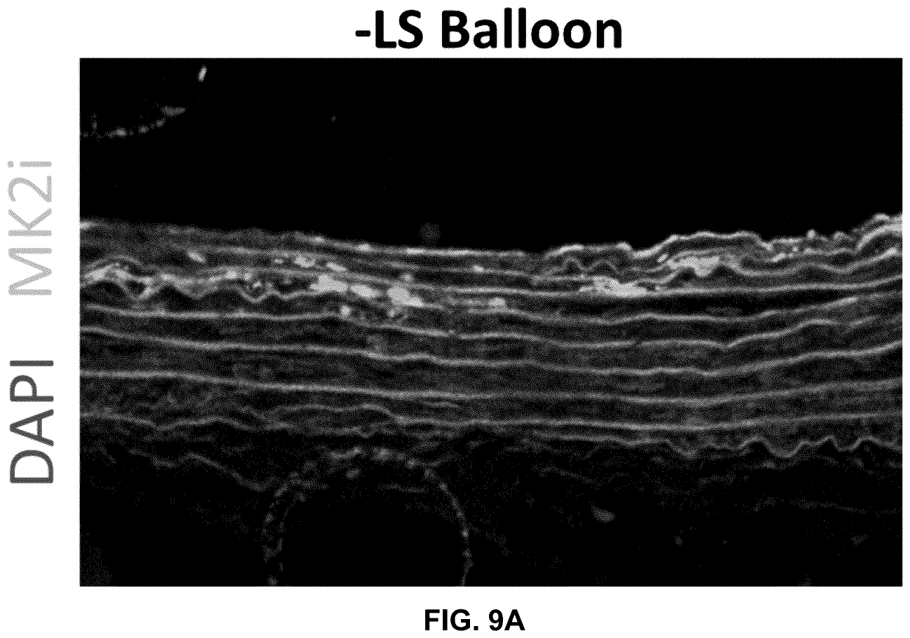

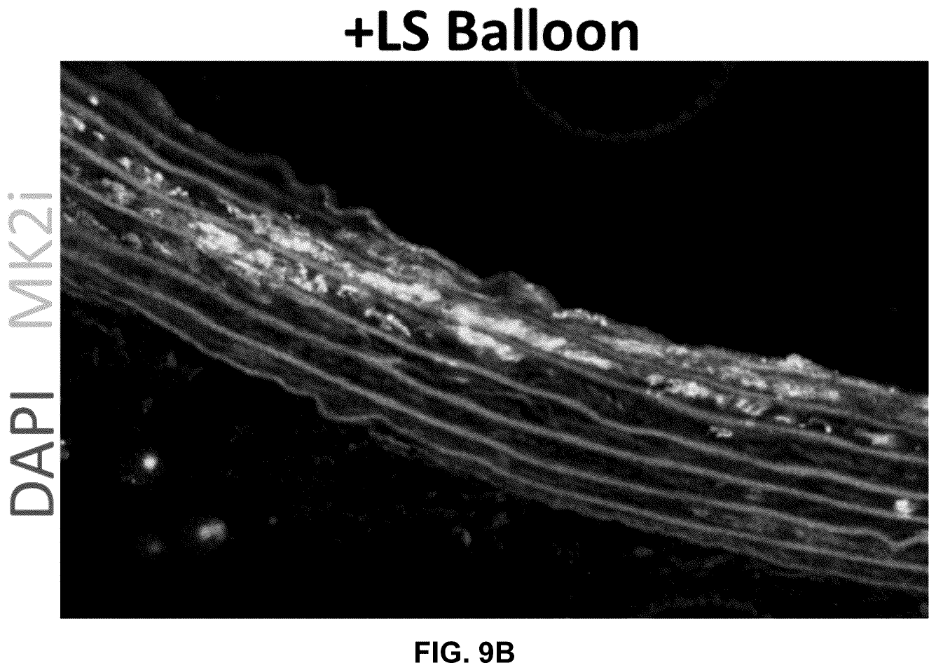

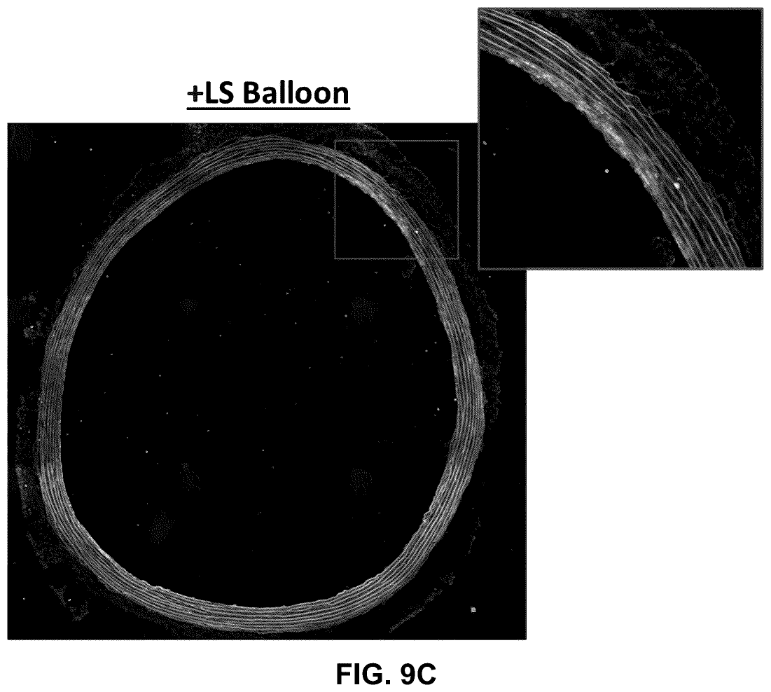

MK2i tissue transfer and cell uptake: Coated balloons (6 layers, 100 μM MK2i, 10 μM PPAA) were inflated in ex vivo rat aortas for 3 minutes and samples were cryosectioned to visualize fluorescent MK2i delivery (FIG. 9A, FIG. 9B, and FIG. 9C). ROIs were drawn around the vessel wall and total MK2i fluorescence was normalized to vessel wall cross sectional area. Additionally, a delivery profile was created by drawing a line (lumen to adventitia) at 8 evenly spaced points around the arterial wall and averaging the intensity along those lines. This created a measurement of MK2i delivery as a function of distance into the arterial wall. Cellular uptake was also measured with flow cytometry, quantifying cellular association of fluorescent MK2i within the treated tissue. In all of these measurements, LS benefited MK2i delivery and cellular delivery.

Therapeutic levels of MK2i achieved in tissue: Coated balloons were compared to the previously established and therapeutically effective method of soaking tissue in a bath of MK2i-nanoparticles (NPs). Ex vivo rat aortas were either treated with a coated balloon (6 layers, 100 μM MK2i, 10 μM PPAA) or soaked in a bath of 50 μM or 10 μM MK2i-NPs for 30 minutes. These samples were then either homogenized and lysed with RIPA buffer or processed for flow cytometry. The fluorescence of the tissue lysate was measured to quantify total MK2i delivery to the tissue, and flow cytometry was used to measure cellular uptake. In both tests, the coated balloons yielded delivery and cell uptake within the range of the two therapeutically effective MK2i-NP treatments.

MK2i retention over time: Treated aortas were incubated in the flow loop bioreactor for 24 hours or 7 days and 10 μm sections were taken to visualize fluorescent MK2i presence in the tissue. Lactosucrose coated balloons appear to have increased retention of MK2i within the tissue at 24 hours and fluorescent MK2i was still detected in the arterial wall after 7 days incubation.

Activity of delivered MK2i: Long-term activity of MK2i was assessed by analyzing cellular proliferation in untreated and treated vessels after 7 d incubation in the flow loop bioreactor. Aortas were balloon damaged and then left untreated or treated with a MK2i+PPAA+LS balloon. After 7 d in the flow loop bioreactor, the samples were cryosectioned, fixed, and stained for PCNA to assess cell proliferation. Positively stained adventitial cells were normalized to total adventitial cell count. Balloon treatment successfully reduced the amount of cell proliferation in the vessel adventitia compared to the untreated, balloon damaged samples.

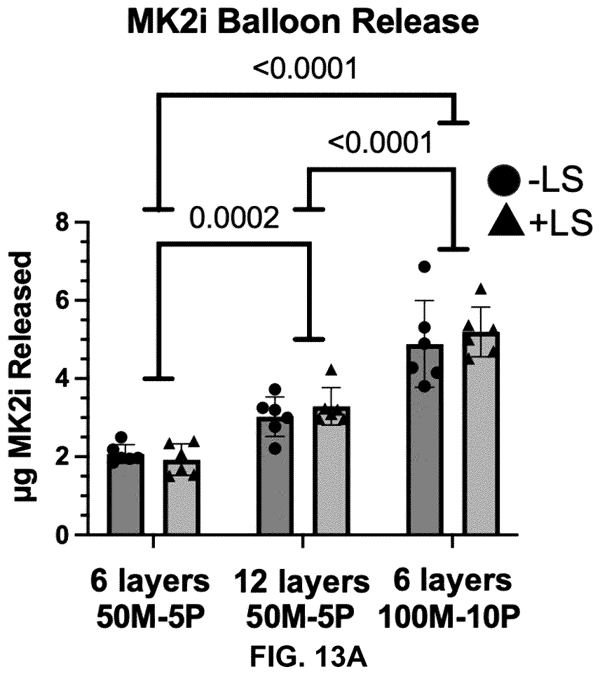

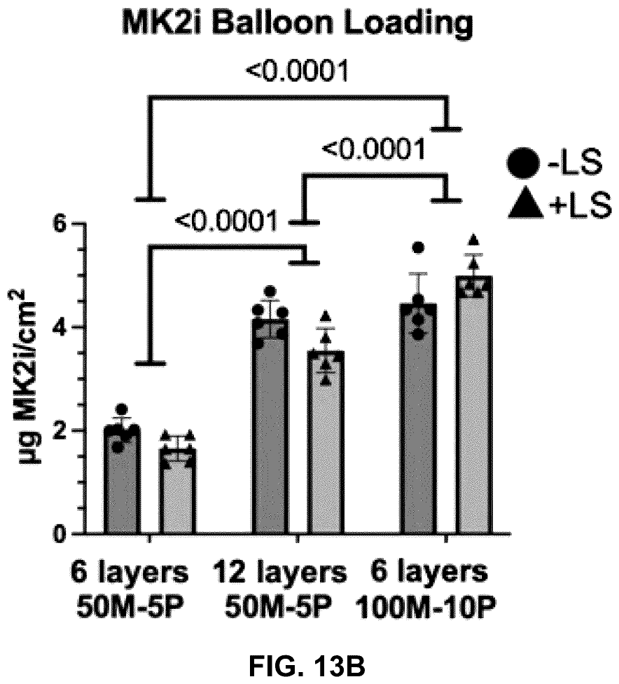

Activity of delivered MK2i: Long-term activity of MK2i was assessed by analyzing cellular proliferation in untreated and treated vessels after 7 d incubation in the flow loop bioreactor. Aortas were balloon damaged and then left untreated or treated with a MK2i+PPAA+LS balloon. After 7 d in the flow loop bioreactor, the samples were cryosectioned, fixed, and stained for Ki67 to assess cell proliferation. Positively stained adventitial cells were normalized to total adventitial cell count. Balloon treatment successfully reduced the amount of cell proliferation in the vessel adventitia compared to the untreated, balloon damaged samples (FIG. 13B).

PPAA v PPA: Aortas were treated with coated balloons+LS with PAA [poly(acrylic acid)] and compared to PPAA. In addition, PPAA-coated balloons load and deliver about twice as much MK2i but have 7× the amount of cellular uptake.

### Materials and Methods for Example 3

Materials: Angioplasty balloons (Boston Scientific) were purchased from Medical Materials, Inc. MK2i peptide (YARAAARQARAKALARQLGVAA (SEQ ID NO:1)) was synthesized by EzBioLab (Carmel, IN) at a scale of 500 mg with a purity ≥95% as determined by mass spectrometry. Antibodies against pCREB (87G3), CREB (86B10), MYH (D8H8), and vimentin (D21H3) were purchased from Cell Signaling Technologies. Antibodies against PCNA (ab29), SM22 (ab14106), CD3 (ab16669), and CD68 (ab125212) were purchased from Abcam. Anti-Fibronectin antibody was purchased from Millipore Sigma (F6140).

PPAA Synthesis: The PPAA polymer was prepared utilizing bulk RAFT polymerization, adapting protocols outlined in Evans et al., “An anionic, endosome-escaping polymer to potentiate intracellular delivery of cationic peptides, biomacromolecules, and nanoparticles,” Nature Communications, Nature Publishing Group, 2019, pp. 1-19, which is incorporated by reference herein in its entirety. The monomer 2-propylacrylic acid (2-PAA) was produced following the approach outlined by Ferritto et al., “POLY(2-ETHYLACRYLIC ACID),” Macromolecular Synthesis, Wiley, New York, 1992, pp. 59-62, which is incorporated by reference herein in its entirety. The 4-cyano-4-(ethylsulfanylthiocarbonyl) sulfanylpentanoic acid chain transfer agent (CTA) was synthesized as described in Convertine et al., “Development of a novel endosomolytic diblock copolymer for siRNA delivery,” Journal of Controlled Release, 2009, pp. 221-229, which is incorporated by reference herein in its entirety. In the polymerization setup, a precise mixture of 2-PAA monomer, 2,2′-azo-bis-isobutyrylnitrile (AIBN) (purified via recrystallization from a methanolic solution) as the initiation agent, and the CTA were combined, maintaining a molar ratio of CTA:AIBN:monomer at 1:1:219; this ratio was calculated to target a final polymer molecular weight of approximately 25,000 g/mol upon complete monomer conversion. The mixture was added to a Schlenk tube equipped with a magnetic stirrer and exposed to three cycles of freezing, vacuum application, and thawing. It was then flushed with nitrogen gas for 30 minutes and kept under nitrogen throughout the polymerization. The reaction was initiated upon heating the mixture to 70° C. The progress of the polymerization was monitored over 72 hours by observing the viscosity and evaluating monomer conversion. The synthesized polymer was exposed to air and diluted into dimethylformamide (DMF), followed by repeated precipitation in cold ethyl acetate (×3) and diethyl ether (×2), and then dried under vacuum overnight. The resulting polymer was dissolved into neutralized phosphate buffer (10 mM) and was dialyzed through a 12-14 kDa MWCO membrane over 2 days, then against 0.01 mM phosphate buffer for an additional day and freeze dried. To characterize the molecular weight and dispersity of the PPAA, gel permeation chromatography (GPC) was employed, utilizing an Agilent 1200 series GPC system equipped with a mini-DAWN T-rex light scattering detector, a variable wavelength detector, and a refractive index detector. The system was configured with three TSKGel Alpha columns (Tosoh) in series and operated at 60° C. The eluent used was HPLC grade DMF with 0.1% LiBr, flowing at a rate of 1 mL/min. Data analysis was conducted using Astra V software (Wyatt Technology), with calibration performed using PMMA and PEG standards supplied by Agilent. Additional verification of the polymer's purity, composition, and molecular weights was carried out through 1H NMR analysis.

Fluorescent MK2i conjugation: Fluorescent MK2i was made using an Alexa Fluor NHS Ester kit (Thermo Fisher). 50 μL of the dye solution was added to 1 mL of 10 mg/mL MK2i in 0.1 μM sodium bicarbonate buffer. The dye-MK2i solution was put on a shaker at RT for 1 hour. The conjugated peptide was then separated and dissolved in PBS using a PD-10 column. Conjugation and fluorescent peptide concentration was quantified by measuring absorbance at 260 and 280 and using the proper correction factor for the Alexa Fluor dye. Alexa 488 (Ex/Em 490 nm/525 nm) was used for flow cytometry studies and Alexa 568 (Ex/Em 578 nm/603 nm) was used for all other studies.

Layer-by-layer balloon coating: Angioplasty balloons were obtained from Medical Materials, Inc. To establish a positively charged base layer, the balloon was air plasma treated (Harrick) for 2 minutes to oxidize the surface and then immediately placed into a 1 w/w % solution of (3-Aminopropyl)triethoxysilane (APTES) in 50% ethanol for 3 hours. To coat the balloon, solutions of MK2i and PPAA were made in methanol at the concentrations indicated. The balloon was then dipped for 3-5 seconds in the PPAA solution and allowed to air dry for one minute before a second dip and dry to establish a thorough coating of PPAA on the balloon. This process was then repeated with the MK2i solution to create a layer on top of the PPAA layer. These steps were repeated to create 6 PPAA/MK2i bilayers on the balloon. Balloon layering was confirmed by using Alexa-568 labeled MK2i (MK2i-568) and visualizing the coating on IVIS and SEM. In some cases, excipient lactosucrose (50 mg/mL) was included in the PPAA coating solution as indicated.

Balloon loading and release quantification: Angioplasty balloons (2.5 mm×20 mm, Maverick OTW, Boston Scientific) were coated with PPAA and MK2i-568 and then soaked in PBS with calcium and magnesium for 3 minutes to measure balloon release. The balloons were then soaked again in DMSO under sonication for 3 minutes to remove any remaining MK2i layers. The fluorescence of the PBS and DMSO solutions was measured on a TECAN Infinite M1000 Pro plate reader and compared to a standard curve of MK2i-568 in PBS and DMSO, respectively.

Rat aorta harvest: Retired breeder Sprague-Dawley rats (Charles River Laboratories) were euthanized with CO₂ and the chest cavity was opened and fascia and connective tissue surrounding the thoracic aorta was carefully removed. Branches from the aorta were cauterized with a Bovie cautery pen (Medline) to allow for vessel pressurization. The aorta was harvested and placed in DMEM (+25 mM HEPES, +4.5 g/L D-glucose, +4 mM L-glutamine, +3% FBS, +1% Antibiotic/Antimycotic) at 37° C. until ready for experimentation.

Aorta treatment: Harvested aortas were damaged with an angioplasty balloon prior to treatment (3×, 15 seconds each, 25% overstretch). Coated balloons were then inserted into the aorta and pressurized for 3 minutes in DMEM (+25 mM HEPES, +4.5 g/L D-glucose, +4 mM L-glutamine, +3% FBS, +1% Antibiotic/Antimycotic), with deflation and reinflation at each minute mark to allow for maximum release and tissue transfer. After 3 minutes, the balloon was deflated and removed. The treated artery was then washed briefly with DMEM to remove excess MK2i and then processed for further experimentation or data collection.

Delivery visualization and quantification: Immediately after treatment, a 3-5 mm segment from the center of the aorta was placed into OCT and frozen for cryosectioning. Samples were cut into 10 μm sections and secured onto slides using ProLong Gold Antifade mounting media with DAPI. After drying, the slides were scanned on a Nikon Eclipse Ti confocal microscope to measure the intensity of the fluorescent MK2i within the arterial cross-section. An ROI was drawn around the outside and inside edges of the vessel using automatic ROI detection on the DAPI channel. Then, total intensity of fluorescent MK2i within the arterial wall was calculated by subtracting the intensity of the inside segment from the intensity of the total segment. The same was done to calculate the area of the arterial wall and then the intensity was normalized to the area for each sample. Additionally, a spatial delivery profile as a function of depth into the arterial wall was created by drawing a line (lumen to adventitia) at 8 evenly spaced points around the arterial wall and averaging the fluorescence intensity along those lines.

Flow Cytometry: After aorta treatment with MK2i-568, samples were incubated in DMEM (+25 mM HEPES, +4.5 g/L D-glucose, +4 mM L-glutamine, +3% FBS, +1% Antibiotic/Antimycotic) for 1 hour to allow for cell uptake. The aortas were subsequently transferred to 500 μL of cold PBS+1% FBS and cut with scissors into small (˜1 mm square) pieces. Digestion media in DMEM was then added to the PBS/tissue tube (500 μL) to achieve a final enzyme concentration of 0.7 mg/mL liberase, 4 mg/mL collagenase, and 0.2 mg/ml DNase, and the tubes were placed in a 37° C. incubator on a shaker for ˜90 minutes or until digestion was apparent. After incubation, the samples were filtered through a 70 μm cell strainer and centrifuged at 500 g for 8 minutes. The resulting cell pellet was resuspended in 200 μL of FACS buffer (PBS+/+, 1% FBS, 2 mM EDTA) and transferred to a 96 well plate for flow cytometry. Cells were read for fluorescence on a Guava easyCyte HT, and the resulting data was processed in FlowJo.

Flow Loop Bioreactor Setup: To prepare the bioreactor system for aorta culture under flow, a sterile 4 mm biopsy punch was used to make holes in sterile 50 ml conical tubes. Connecting tubes were sterilized with ethanol and washed through with 3% hydrogen peroxide followed by DI water. Treated aortas were hung inside each 50 ml tube and stretched to 30% of their basal level to simulate physiological tension. The media used was based on methods described in Wang et al., “An ex vivo physiologic and hyperplastic vessel culture model to study intra-arterial stent therapies,” Biomaterials 275 (2021), which is incorporated by reference herein in its entirety. Flow loop media was created by mixing 50% DMEM (+25 mM HEPES, +4.5 g/L D-glucose, +4 mM L-glutamine) and 50% VascuLife growth media (VWR) supplemented with 1% Antibiotic/Antimycotic and 30 g/L dextran to increase the viscosity of the media to be similar to blood. Flow loop media was further supplemented to 20% FBS to promote VSMC phenotype switching within the arteries. Aortas were connected to tubing with plastic cannulas and secured with sterile sutures. The flow rate was set to 6 mL/minute to create 4-5 dyn/cm² shear stress, which is similar to that of atherosclerotic arterial regions. Doriot's equation was used to calculate the ideal flow rate with a set viscosity of 0.043 dyn s/cm² based on an average arterial diameter of 1.8 mm, determined from histological sections. The media flowing through the aorta was replaced every other day, while 30% of the media circulating around the aorta was replenished every day.

MTT Viability: Aortas incubated in the flow loop bioreactor were taken out and a small ring from each was further incubated in 400 μL PBS with 2.5 mg/mL (3-(4,5-dimethylthiazol-2-yl)-2,5-diphenyltetrazolium bromide (MTT) for 30 minutes. After incubation, the rings were transferred to 400 μL of Cellosolve to dissolve the formazan crystals for 1 hour on a rocker at RT. Then, 100 μL of the Cellosolve solution was transferred to a 96 well plate for each sample. The absorbance at 590 nm was read on a TECAN Infinite M1000 Pro plate reader and normalized to the mass of the aorta ring. Relative viability was determined by comparing incubated samples to fresh rat aorta samples.

Western Blotting for pCREB inhibition: Acute (1 hr) inhibition of cAMP-response element binding protein (CREB) phosphorylation was assessed by in rat aorta samples divided in half. One half underwent initial balloon damage, followed by administration of vehicle control (PBS for convective delivery, uncoated balloon for balloon delivery) for 3 minutes. The other half of each sample underwent a DCB MK2i treatment. The samples were incubated for 1 hr after balloon damage/treatment in DMEM (+25 mM HEPES, +4.5 g/L D-glucose, +4 mM L-glutamine, +3% FBS, +1% Antibiotic/Antimycotic) before being processed for western blotting. For extended CREB inhibition, aorta samples were incubated in the flow loop for 24 hours, 3 days, or 7 days after treatment. Each aorta was then divided in half and transferred to flow loop media with or without 60 μM LPA (or 240 μM LPA for 7-day samples) for 2 hours. The samples were then flash frozen in liquid nitrogen. Frozen aortas were homogenized with a tissue pulverizer and lysed in RIPA buffer with protease and phosphatase inhibitors (Roche). The tissue homogenate was vortexed for 15 minutes at 4° C. and then centrifuged at 12,000 g for 15 minutes at 4° C. to collect the supernatant. Protein concentration was quantified using a BCA Assay kit (Pierce). Protein (40 μg) was resolved on 4-20% SDS-PAGE gels and transferred to a nitrocellulose membrane using the Invitrogen iBlot 2. Blots were incubated in primary pCREB antibody (1:1000) overnight at 4° C. and incubated in the secondary antibody (Li-Cor 926-32211, 926-32210, 1:5000 dilution) for 1 hr at room temperature. Blots were imaged on a LiCor Odyssey Fc. The blots were then stripped and stained for CREB using the same method.

MK2i Delivery and Retention Quantification: Treated aorta samples were incubated in the flow loop bioreactor and then digested in the same digestion medium used for flow cytometry. Aortas were digested in 20× the volume of digestion media relative to their mass. Once the tissue was sufficiently digested, the fluorescence (Ex 578, Em 603) was measured on a TECAN Infinite M1000 Pro plate reader and compared to a standard curve created using a digested untreated aorta.

Muscle Baths: Aortic rings were cut (˜2 mm thick) and suspended in the muscle bath (Radnoti) containing bicarbonate buffer (120 mM NaCl, 4.7 mM KCl, 1.0 mM MgSO₄, 1.0 mM NaH₂PO₄, 10 mM glucose, 1.5 mM CaCl₂, and 25 mM Na₂HCO₃, pH 7.4), equilibrated with 5% CO₂ at 37° C. at a resting tension of 1 g, manually stretched to three times the resting tension, and maintained at resting tension for an additional 1 hour. This produced the maximal force tension relationship. Next, the rings were primed with 110 mM of potassium chloride (with equimolar replacement of sodium chloride in bicarbonate buffer) to determine functional viability. Rings were then stimulated with escalating doses of phenylephrine (PE) to determine contractile responses to agonist. Force measurements were obtained using the Radnoti force transducer (model 159901A, Radnoti) interfaced with a PowerLab data acquisition system and LabChart software (AD Instruments Inc, Colorado Springs, CO). Contractile responses were defined by stress, calculated using force generated by tissues as follows: stress (×10⁵ N/m²)=force (g)×0.0987/area, where area=wet weight (mg)/at maximal length (mm)]/1.055. Significance was determined by performing nonlinear regression on the data sets and comparing the fit curves using an ANOVA with the EC50 and SEM values.

Immunofluorescence: Rat aortas were balloon damaged and either treated or left untreated before incubation in the flow loop bioreactor for 7 days. Aorta samples were then fixed with 10% neutral buffered formalin and embedded in paraffin. Samples were cut into 5 μm sections and allowed to dry on slides overnight. The slides were then baked in a 60° C. oven for 1 hour, rehydrated with xylene, 100% EtOH, and 95% EtOH washes, and antigen retrieval was performed in pH 6 citrate buffer at 97° C. for 20 minutes in an Epredia PT Module. For immunofluorescence, slides were treated with serum-free protein block (Daco) for 20 minutes followed by fluorescent blocking buffer (Thermo) for 30 minutes. The slides were then incubated in the primary antibody for 1 hour followed by the secondary antibody for another hour, and coverslips were secured onto each slide using ProLong Gold Antifade mounting media with DAPI. Slides were scanned using a Nikon Eclipse Ti confocal microscope. MYH, VIM, PCNA, SM22, CD68, and FN antibodies were used at a 1:1000 dilution, CD3 antibody was used at 1:500. Secondary antibodies (Invitrogen) were used at a 1:1000 dilution.

Rat carotid artery balloon injury model: Male Sprague Dawley rats (400-550 g) were anesthetized with 3% isoflurane and the area from the chin to the top of the sternum was shaved. The surgical site was cleaned with iodine and then an incision was made in a straight line from the chin to the sternum. Glandular tissue, fascia, and muscle was blunt dissected away and retracted to expose the left carotid artery. The proximal common carotid artery was temporarily clamped to cut off blood flow, and the distal external carotid was tied to prevent backflow. Then, on the external carotid artery branch, a small arteriotomy incision was made about ¼-⅓ of the circumference of the vessel. Through this incision, the deflated angioplasty balloon (1.5 mm×12 mm, Emerge OTW, Boston Scientific) was inserted up to the arterial clamp. Then, the clamp was released, and the balloon was inserted further down to the aortic arch. Once the balloon was in place, it was fully inflated with a sterile saline syringe and gently withdrawn with rotation to damage the vessel. When the balloon was close to the arteriotomy incision, it was deflated and inserted again. The inflation and removal were repeated 2 more times to fully damage the artery. Prior to full removal of the balloon, a suture was loosely tied around the external carotid artery proximal to the arteriotomy incision. After 3 rounds of balloon insertion and damage, the balloon was deflated and removed, and the suture around the external carotid artery was quickly tied tight to prevent bleeding from the incision. In animals that were in the treatment group, the carotid artery was instead clamped again prior to balloon removal. A MK2i+PPAA coated balloon (1.5 mm×12 mm, Emerge OTW, Boston Scientific) was then introduced into the arteriotomy incision and inserted down to the area of balloon damage. This balloon was then inflated at the damaged site for 3 minutes, with deflation and re-inflation at each minute mark to aid in drug transfer. After this treatment time, the balloon was deflated and removed, and the external carotid was tied to prevent bleeding. Any other sutures and ligations were then removed, and branching arteries were checked to ensure blood flow. Two weeks after surgery, the rats were euthanized, and the carotid arteries were removed and fixed in 10% neutral buffered formalin for histology and immunofluorescence staining.

Statistics: Statistical significance for experiments with more than 2 groups was determined using one-way ANOVA tests with Tukey's post hoc test and experiments with 2 groups used Welch's t test unless otherwise noted. For optimization of balloon loading, significance was determined using a two-way ANOVA and comparisons between groups. For 1 hour pCREB western pairwise comparisons, significance was determined with ratio paired t tests. Significance for the muscle bath contractility test was determined with a one-way ANOVA and Tukey's post hoc test on IC50 values determined from linear regression models of the PE vs contraction curves. Analyses were performed in GraphPad Prism 10 software. Results are presented as arithmetic mean±SD with P values as indicated in the figures or figure legends. P values of less than 0.05 were accepted as significant.

### Example 3

### Mk2i Delivery Via Drug Coated Balloons

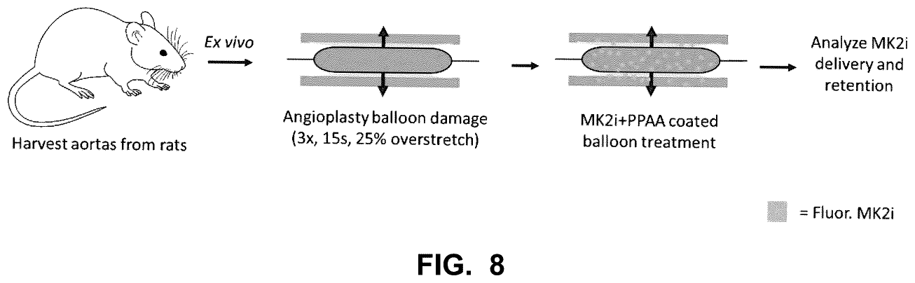

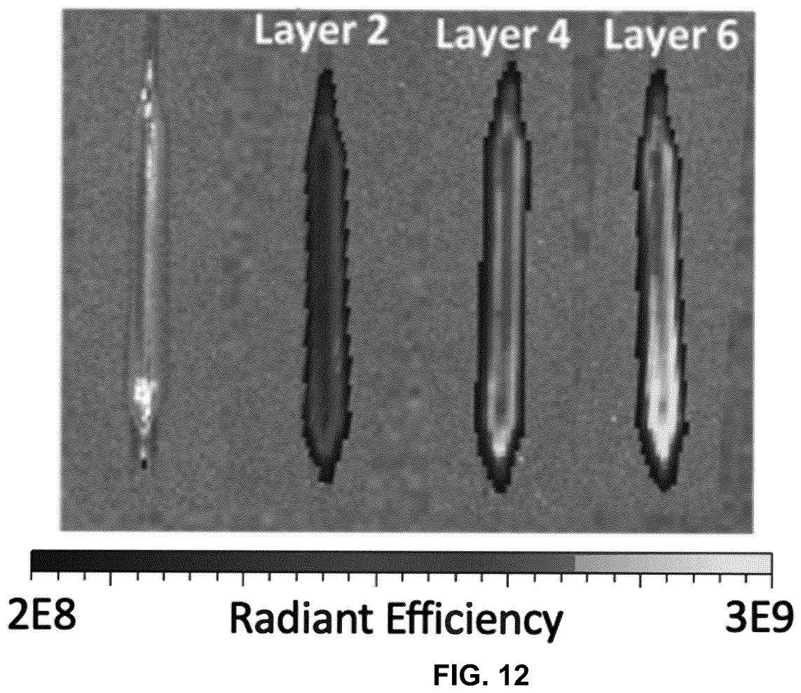

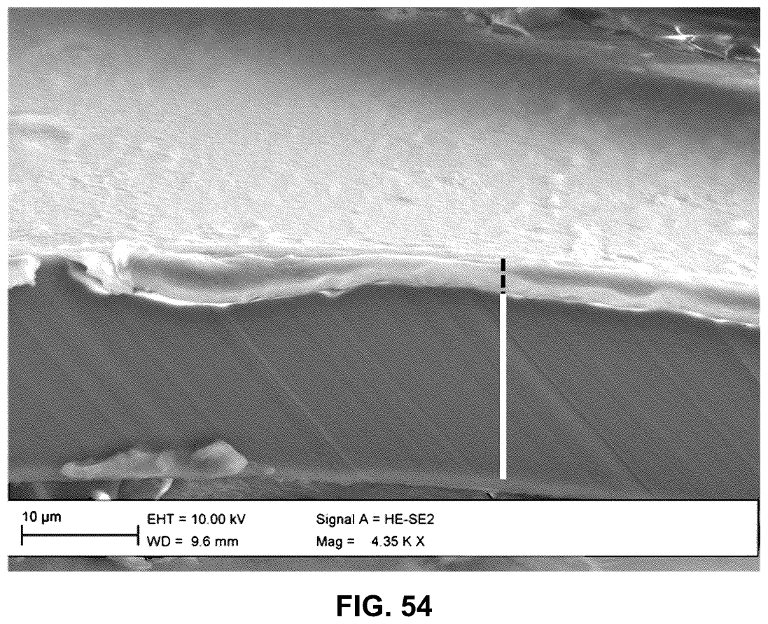

Layer-by-layer MK2i+PPAA coating of angioplasty balloons to maximize loading and release: MK2i+PPAA LbL drug coated balloon methods were tested in ex vivo rat aortas (FIG. 8). To create a coating of MK2i+PPAA on the surface of a plain angioplasty balloon, a LbL method was implemented (FIG. 4). The balloon was first air plasma cleaned and soaked in an APTES solution to functionalize the surface and create a positively charged base layer of amines. Then, the balloon was dip coated using alternating solutions of PPAA and MK2i to create layers of positive and negative charges. Lactosucrose (LS) was tested as an excipient within the PPAA layers to potentially help with tissue transfer and dispersion of the coating upon deployment in tissue. Using fluorescently labeled MK2i, layering was confirmed using IVIS (FIG. 12), and the coating was visualized with SEM (FIG. 54).

Loading and release of MK2i from the surface of the balloons were quantified (FIG. 13A and FIG. 13B). Initial coating with 6 layers using solutions of 50 μM MK2i and 5 μM PPAA (6L-50M-5P) yielded 1.5-2 μg/cm² of MK2i loading. Increasing the number of layers (12L-50M-5P) and increasing the concentration of the loading solutions (6L-100M-10P) both increased the peptide loading, with 6L-100M-10P balloons (4.5-5 μg/cm²) showing the best MK2i loading. For each of the loading conditions, the variance between samples was fairly low, showing that the dip coating method yielded reproducible coatings. When balloons were tested for release in PBS, 12L-50M-5P balloons did not show as efficient release of the MK2i from the balloon surface, potentially due to the increased interactions between layers. The higher loading and release of the 6L-100M-10P method led to coating balloons with this strategy for the remaining studies. These balloons will be simply referred to from here as ±LS DCBs.

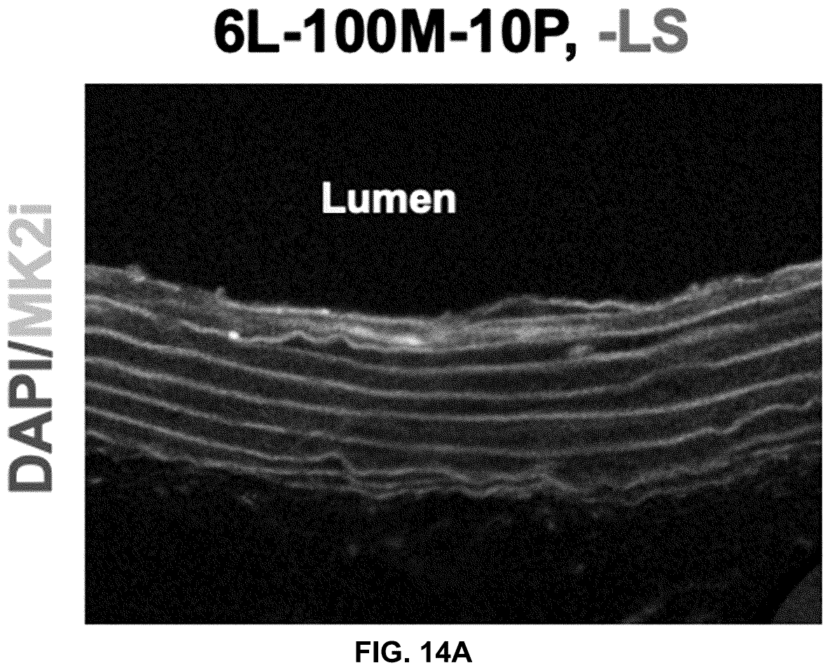

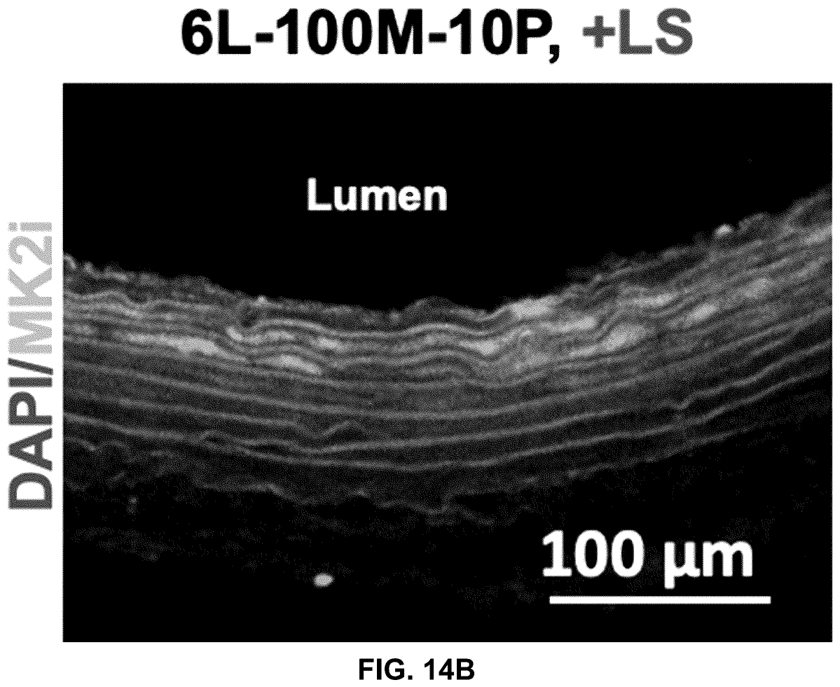

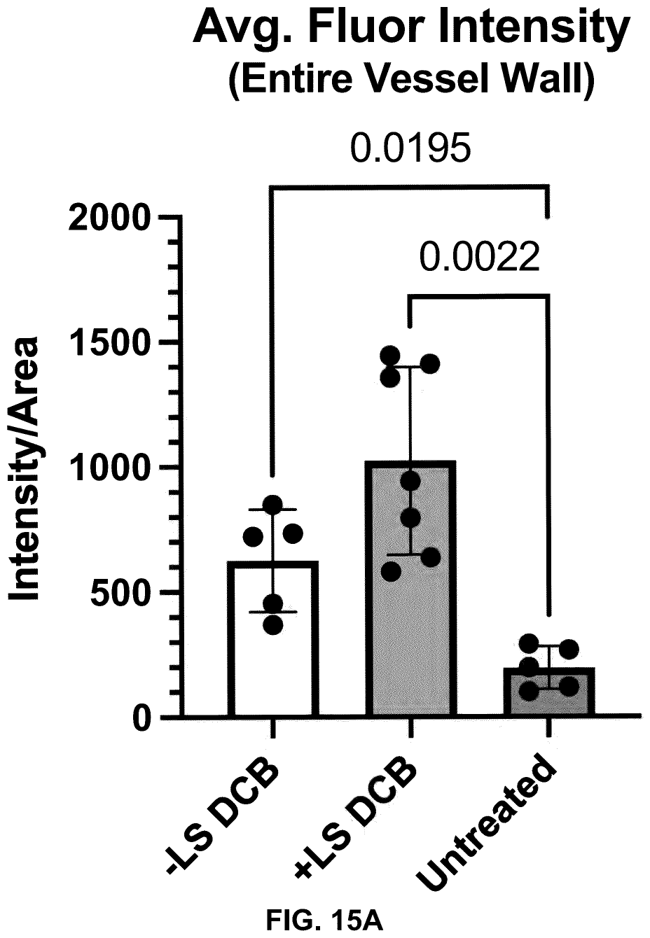

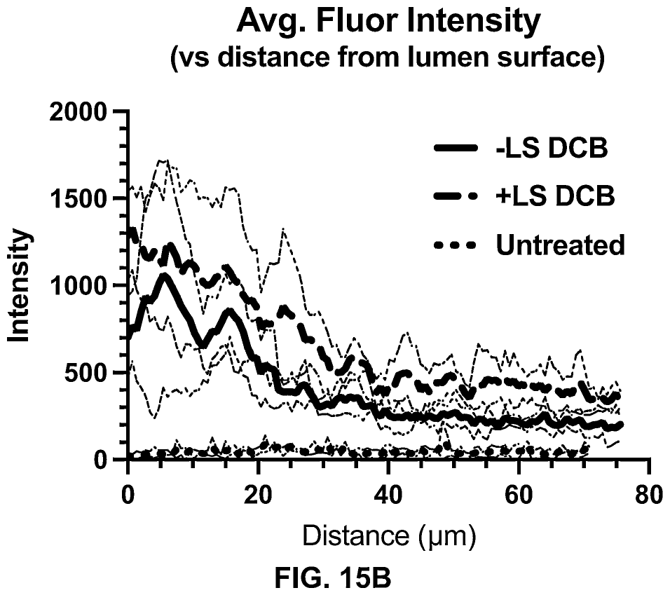

Although addition of LS to the PPAA layers did not yield any differences in loading and release in PBS, a greater effect of LS inclusion was observed when these balloons were used to treat rat aortas ex vivo (FIG. 14A and FIG. 14B). The amount of MK2i delivered and the level of vessel wall penetration was measured for aortas treated with coated balloons with or without LS (FIG. 15A). LS increased MK2i transfer to the tissue and had a modest effect on increasing the depth of delivery as shown by the intensity profiles. Most critically, LS addition significantly increased the intracellular uptake of MK2i, as shown by flow cytometry on isolated SMCs 1 hour after treatment (FIG. 15B).

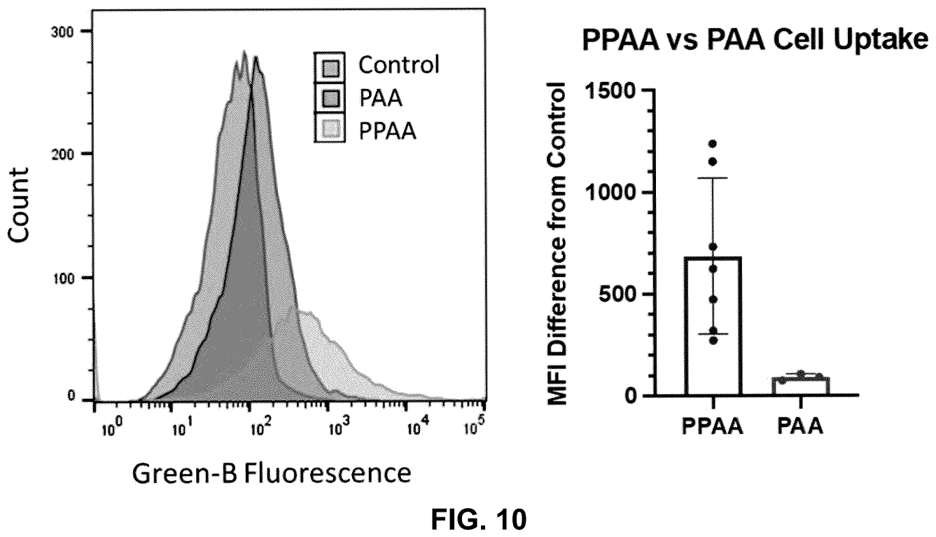

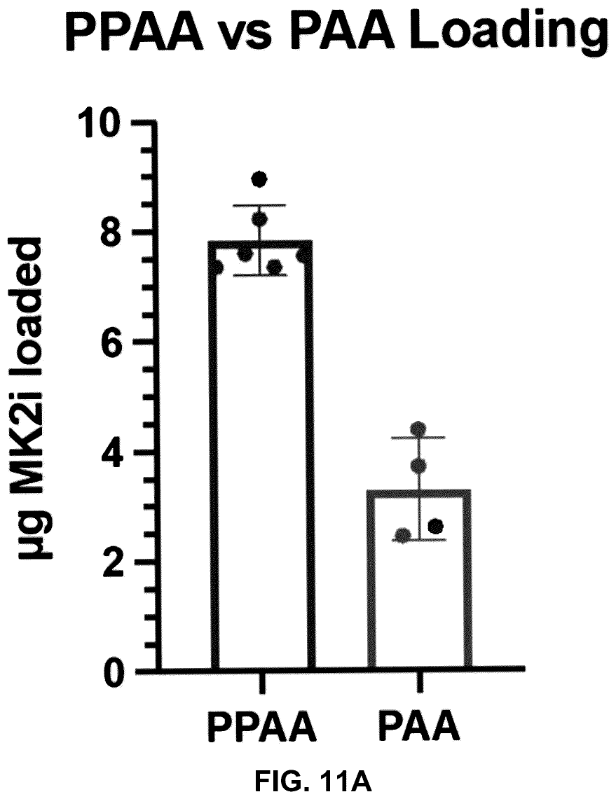

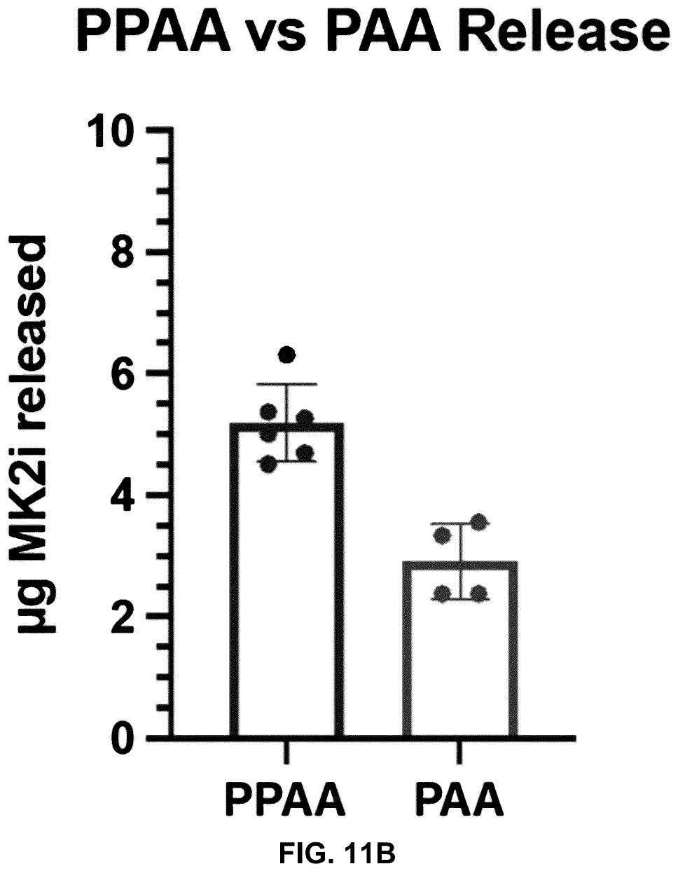

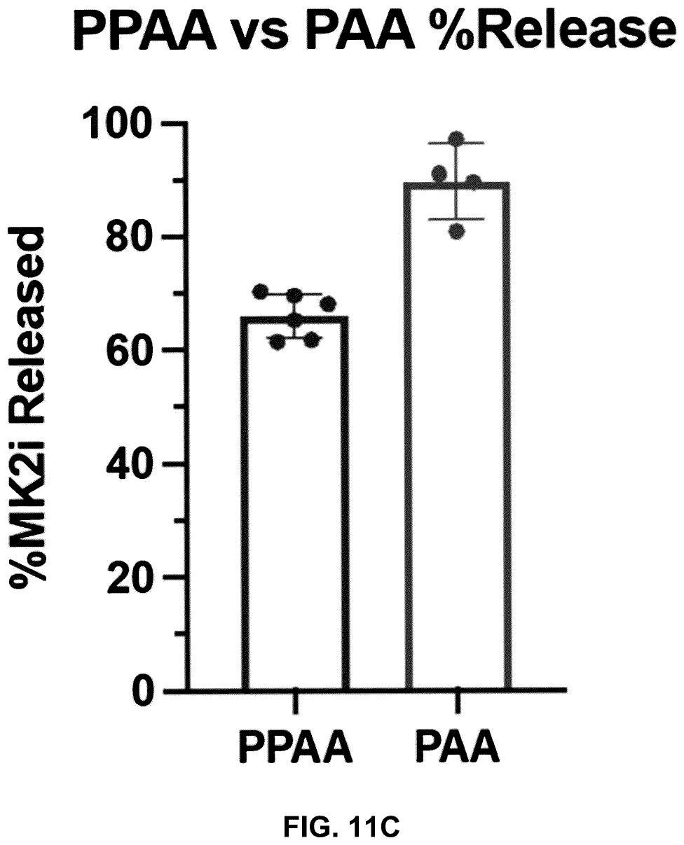

PPAA and similar polymers can increase cell internalization of MK2i and similar peptides in vitro, with the hydrophobic side group of the polymer being critical in interaction with cell membranes and, combined with the carboxylic acid group, promoting pH-dependent escape from endolysosomal vesicles. Here, the PPAA structure was probed in tissue-level cell internalization relative to use of other analogous polyanions in the LbL balloon coating (FIG. 10, FIG. 11A, FIG. 11B, and FIG. 11C). PPAA was compared to poly(acrylic acid) (PAA), a charged polymer that lacks the hydrophobic propyl side chain present on PPAA. While PAA also effectively loaded and released MK2i from the balloons, cell internalization of MK2i delivered into rat aortas with PAA+LS DCBs was negligible compared to the significant uptake realized in tissues treated with PPAA+LS DCBs. These data confirm the criticality of the use of PPAA in the LbL DCB design.

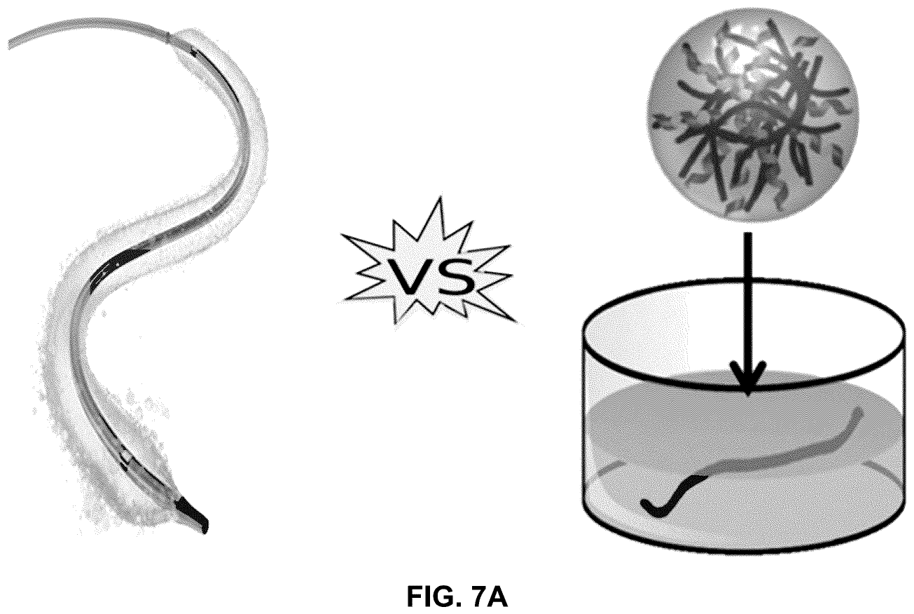

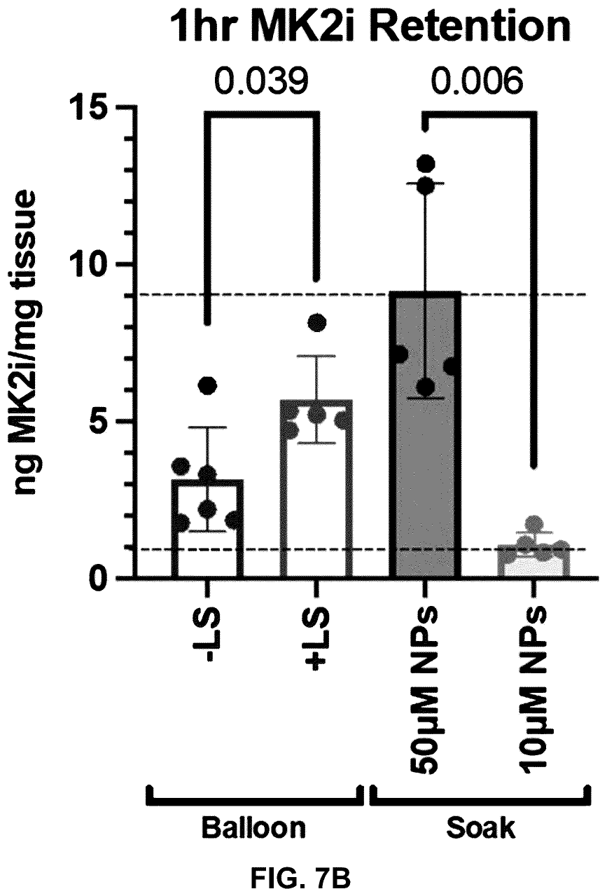

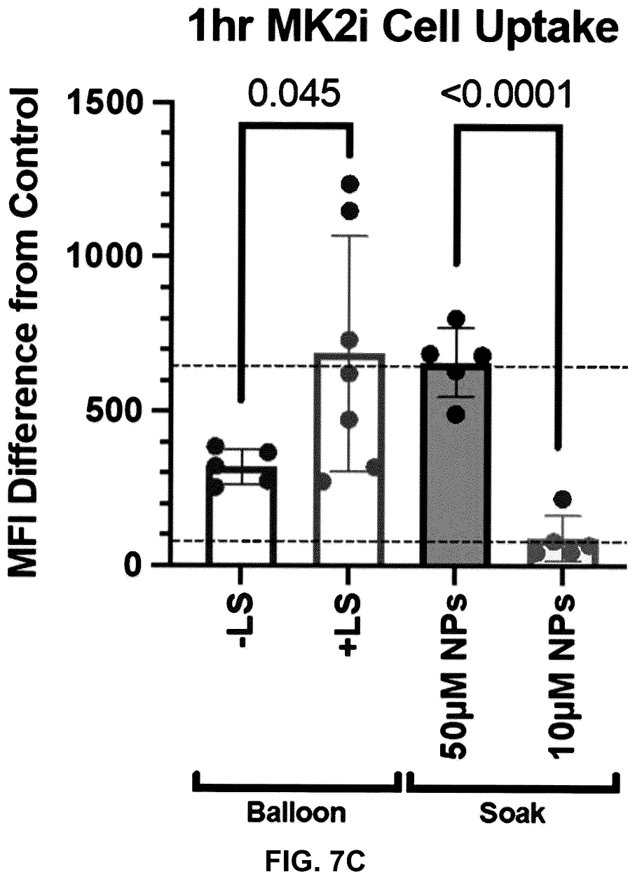

Comparison to topical MK2i-NP delivery. It was next sought to benchmark the relative intracellular delivery of DCBs with an in-house standard. To accomplish this, cell uptake of MK2i was compared to a method of topical MK2i-NP delivery. Incubation with MK2i-NPs at concentrations of 10 μM and 50 μM peptide previously prevented IH and SMC phenotype switching in venous tissues and in vitro. Rat aortas were incubated in a solution of MK2i-NPs for 30 minutes and digested to analyze cell uptake after 1h incubation. These two doses give a range within which the DCB delivery method falls (FIG. 7A, FIG. 7B, and FIG. 7C). Therefore, these data suggest that DCB delivery achieves delivery of therapeutically relevant levels of MK2i peptide.

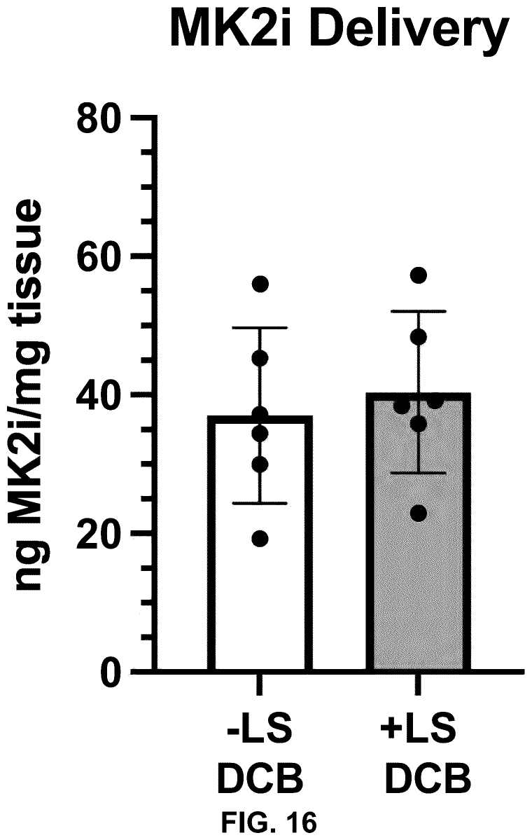

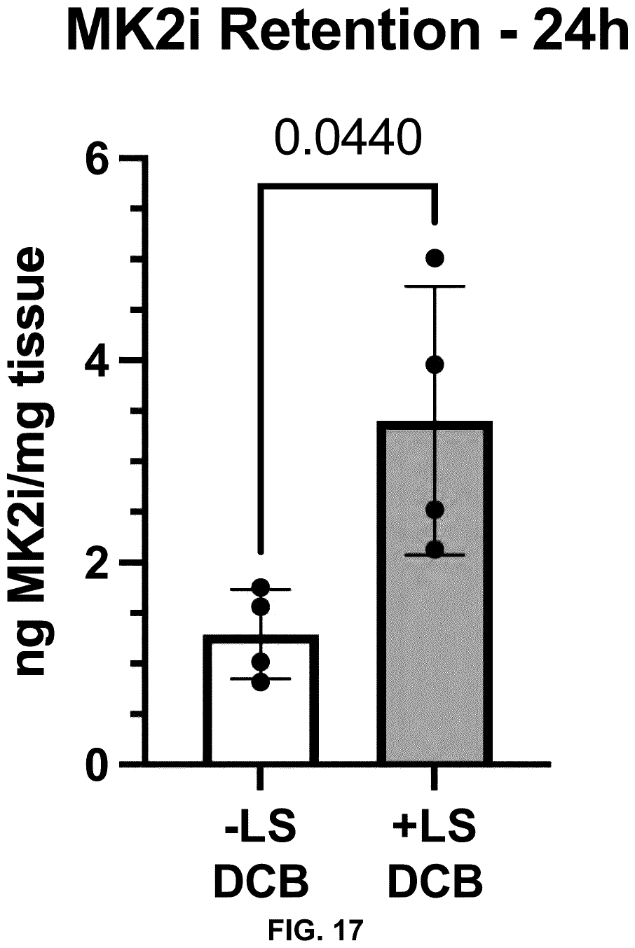

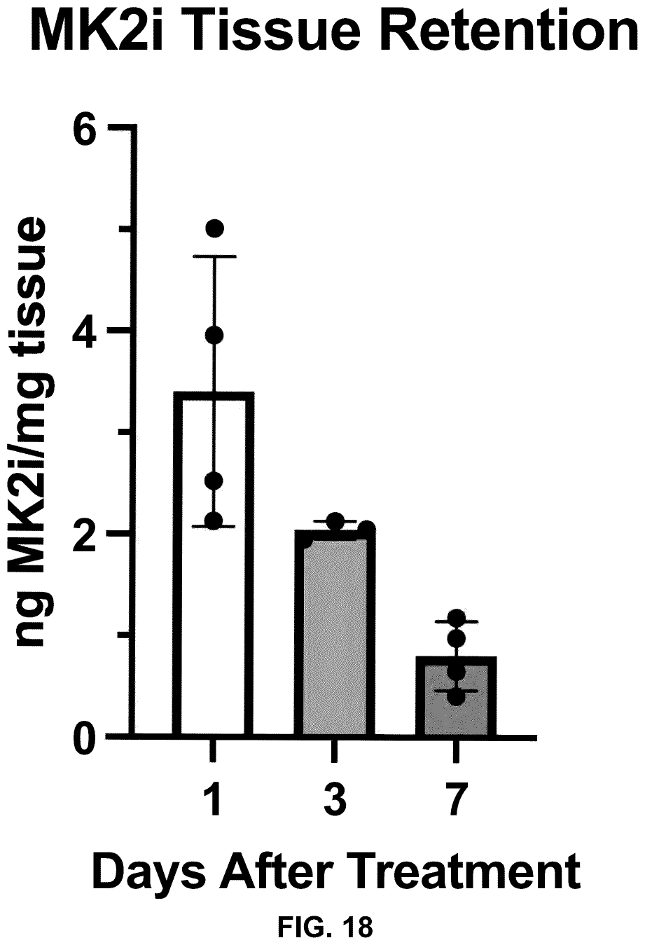

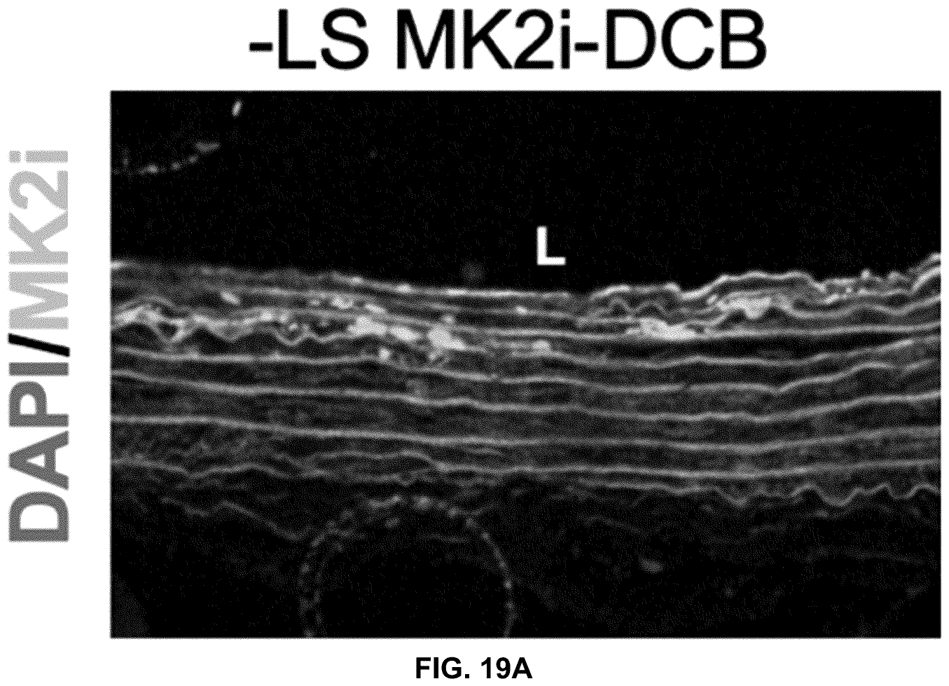

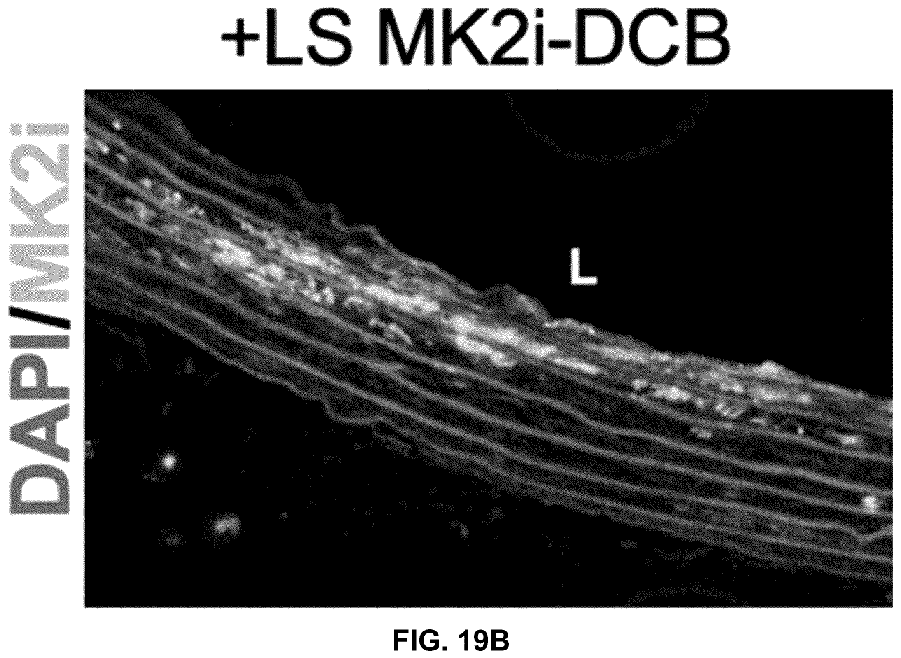

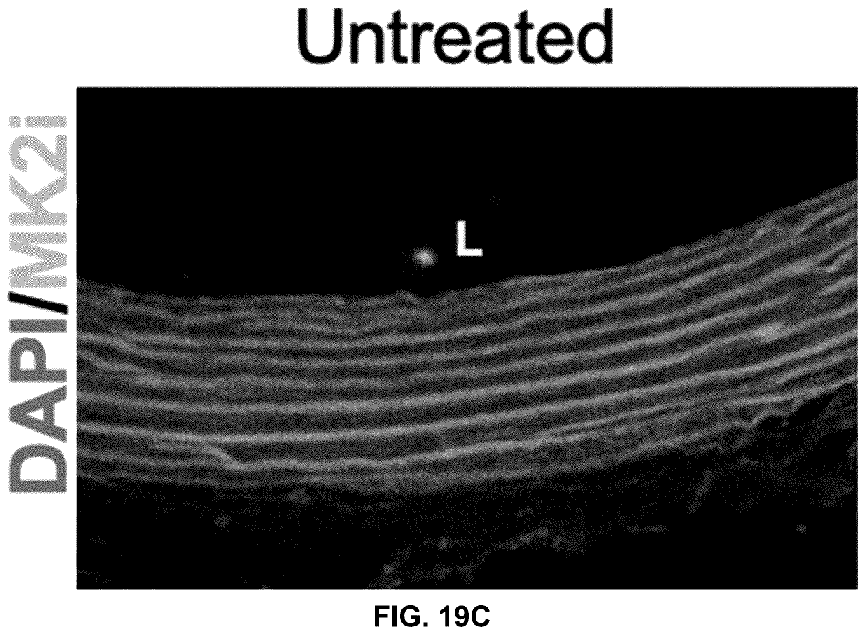

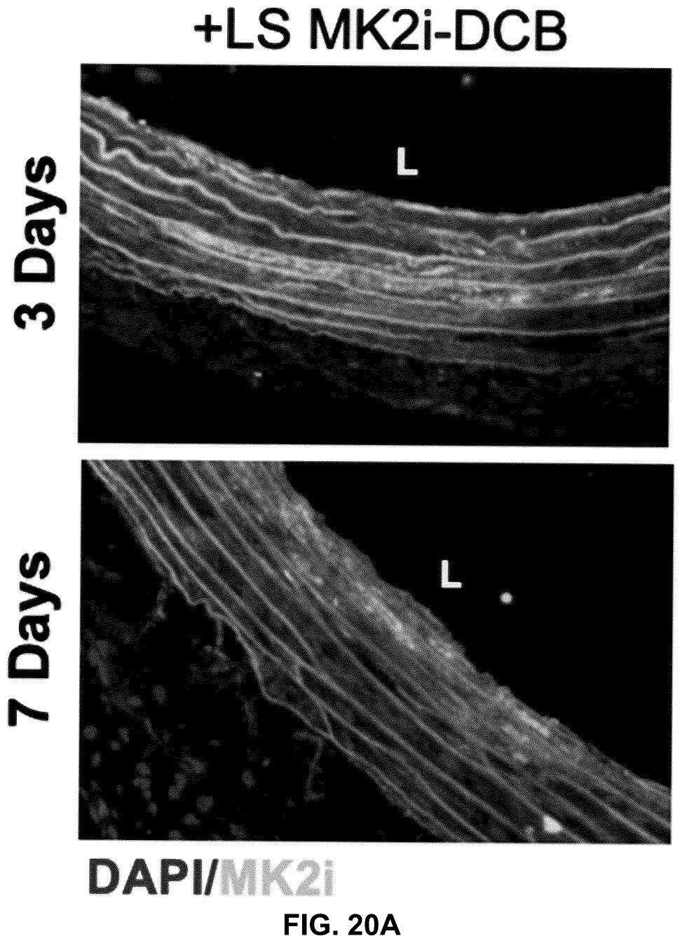

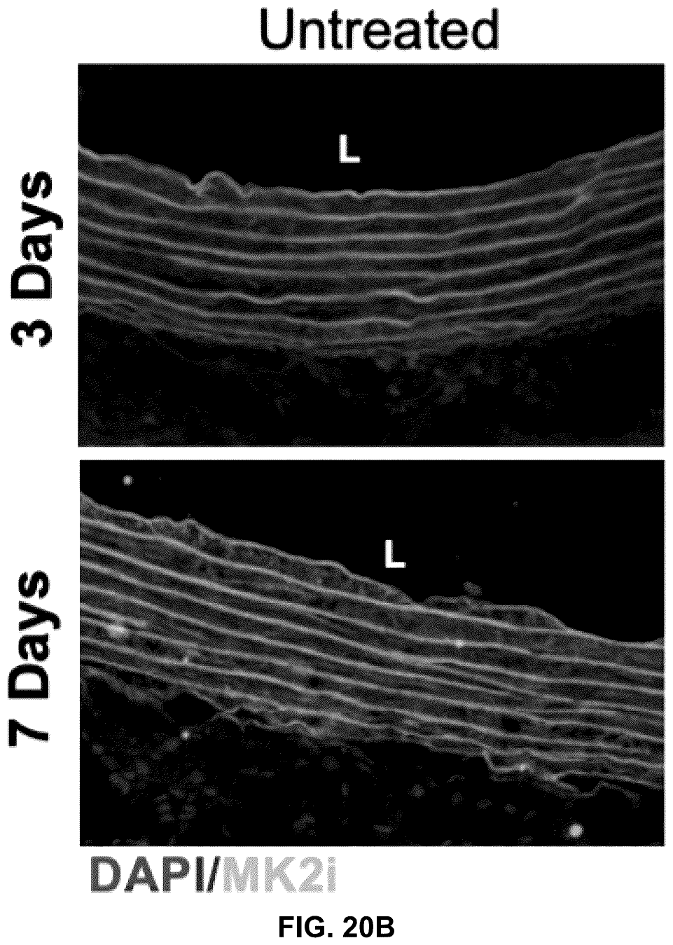

Pharmacokinetics of balloon delivered MK2i: To study the arterial retention of MK2i over time under flow, a bioreactor was constructed to house the aortas under physiological conditions. The viability of aortas housed in this bioreactor was confirmed at 24 hour and 7-day time points using the MTT viability assay, with no significant drop in viability after one week. Initial tissue MK2i concentration was calculated from digested aortas immediately after treatment (FIG. 16). Using this bioreactor setup, the amount of MK2i in the vessel wall was observed at 24 h post-treatment for all delivery strategies and formulations (FIG. 19A, FIG. 19B, and FIG. 19C) and quantified from digested tissues (FIG. 17). +LS DCBs had significantly more retention at 24 hours than the other delivery formulations. Therefore, this delivery method was assessed for longer-term MK2i retention at 3 days and 7 days in the flow loop bioreactor. MK2i was still present within the arterial wall at these later time points (FIG. 20A and FIG. 20B). Tissue MK2i concentration at these time points showed a steady decrease, but still a significant presence of the peptide within the tissue (FIG. 18). MK2i presence up to 7 days is important for inhibition of the acute stress response, as it has been shown that the majority of cellular proliferation takes place within the first week after vascular interventions.

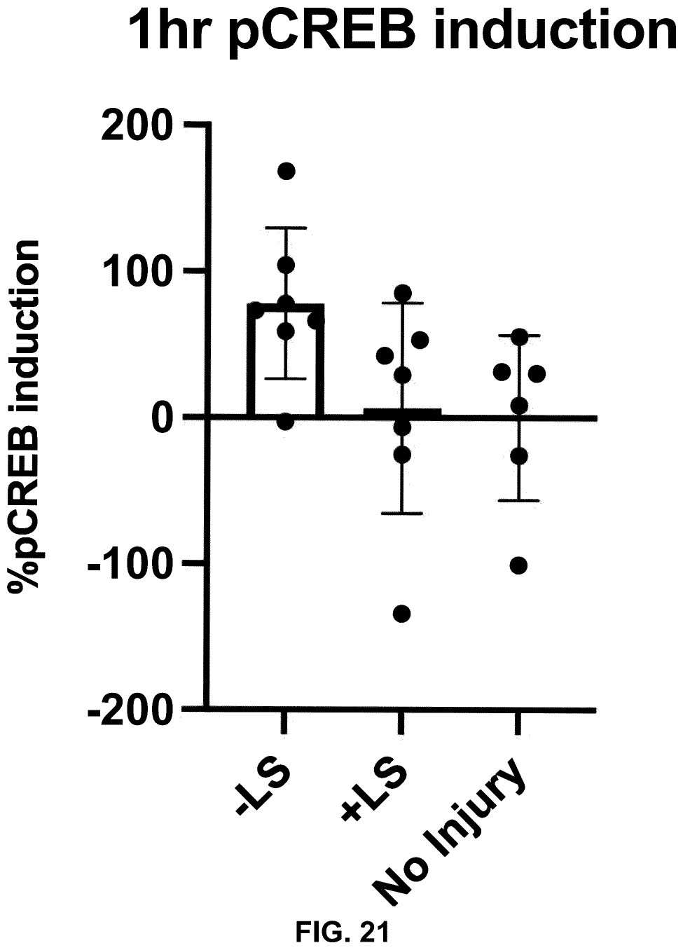

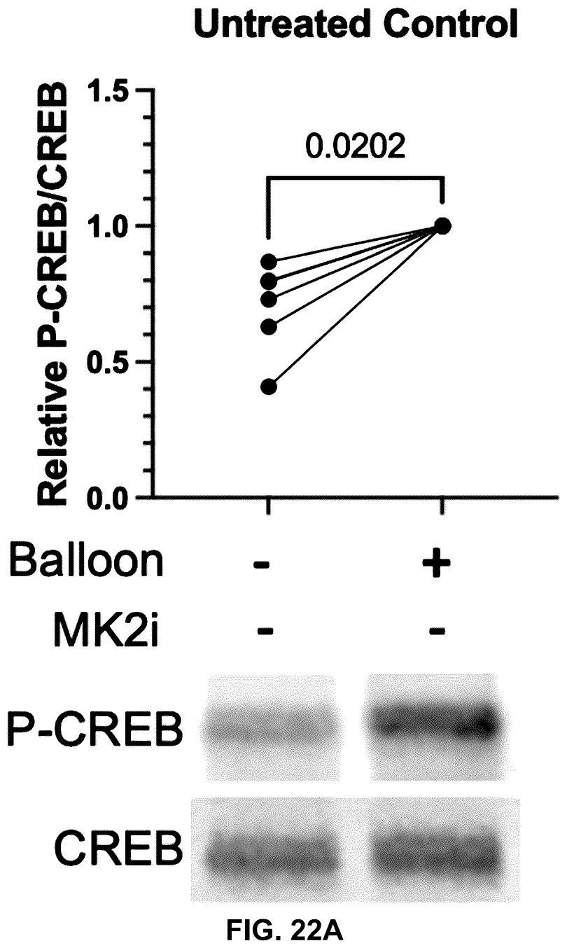

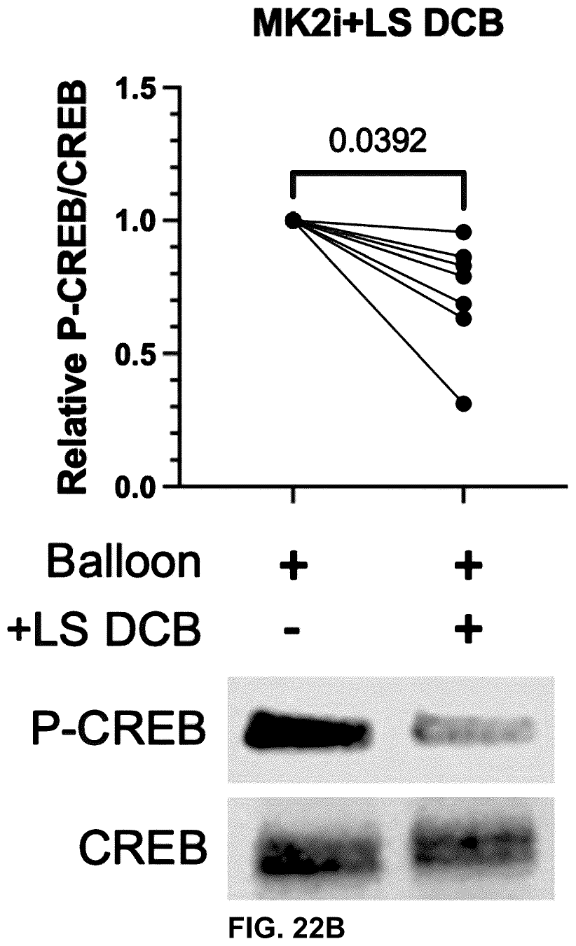

MK2i blocks phosphorylation of CREB over time in treated rat aortas: The ability of delivered MK2i to block MK2 signaling was determined by measuring the downstream phosphorylation of direct MK2 substrate cAMP response element-binding protein (CREB), a transcription factor involved in VSMC proliferation. To measure inhibition of acute CREB phosphorylation, each rat aorta was divided in half—one half was balloon damaged and vehicle treated while the other half was balloon damaged and then treated with MK2i+PPAA. After 1h static incubation, tissue lysates were prepared and levels of pCREB were analyzed by western blot. The relative level of pCREB induced by balloon damage was significantly reduced in MK2i-NP treated and +LS DCB treated aortas and was similar to the level in a control untreated, undamaged aorta (FIG. 21). Pairwise comparisons of aorta halves showed that balloon damage significantly increased pCREB levels compared to an untreated, undamaged aorta segment, and treatment with +LS DCB inhibited that pCREB induction (FIG. 22A and FIG. 22B). The inclusion of LS in the DCB formulation has a beneficial effect on MK2i delivery and bioactivity, and therefore DCB formulations in studies from this point used LS and simply will be referred to as “MK2i DCBs.”

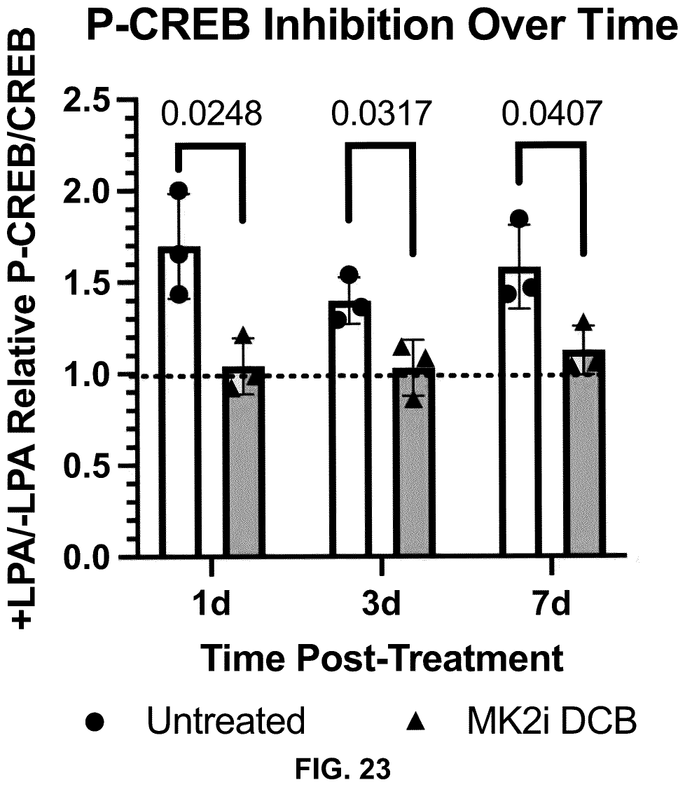

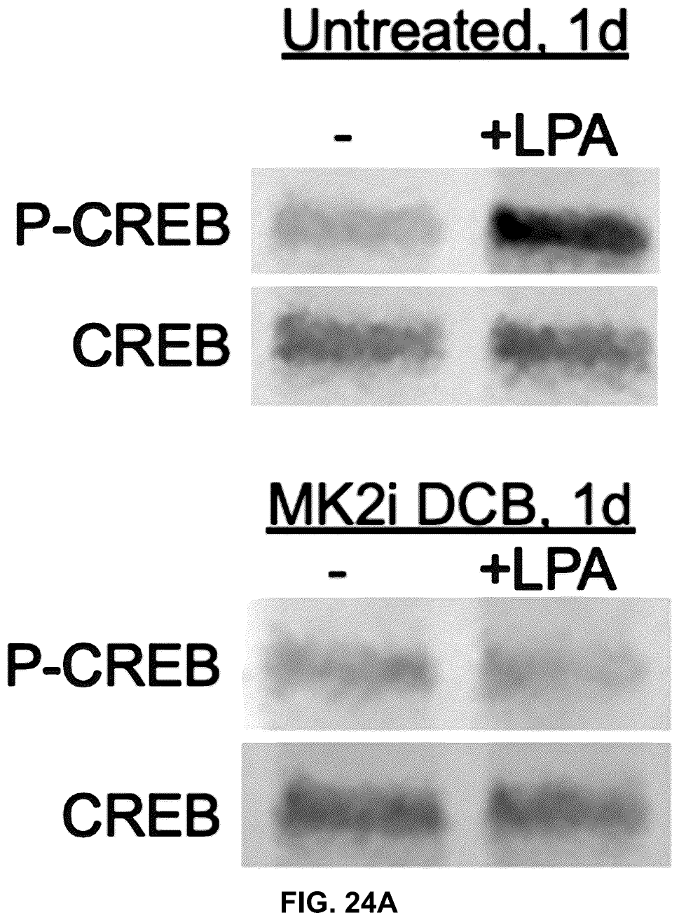

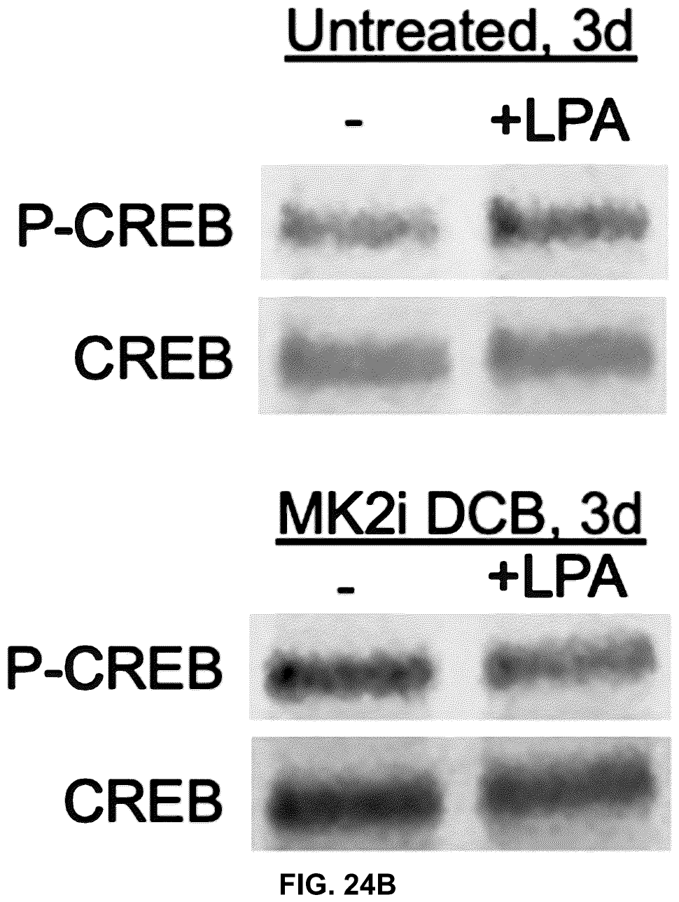

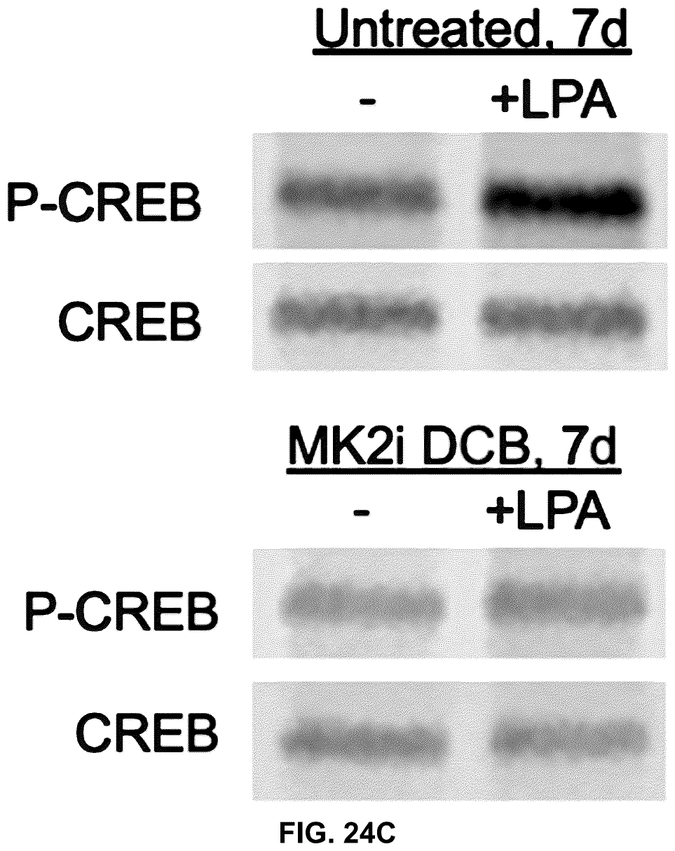

The pharmacodynamics of MK2 signaling inhibition were next monitored over time. To measure MK2i activity after incubation in the flow loop, untreated or MK2i-treated aortas were divided in half—one half was stimulated with lysophosphatidic acid (LPA), which has been shown to stimulate VSMC CREB phosphorylation in previous studies, while the other half was left unstimulated. The amount of pCREB induction was compared between the two halves for each treatment group at 24 hours, 3 days, and 7 days (FIG. 23, FIG. 24A, FIG. 24B, and FIG. 24C). LPA was shown to stimulate an increase in pCREB in the untreated samples at each time point, while MK2i-NP and MK2i DCB treatment effectively blocked LPA-stimulated CREB phosphorylation at all time points, having no significant difference in pCREB levels from samples not stimulated with LPA. These results show the potent effect of intravascular MK2i+PPAA delivery on MK2 inhibition and corroborates the activity of the MK2i peptide that was observed in the artery after 7 days in dynamic culture.

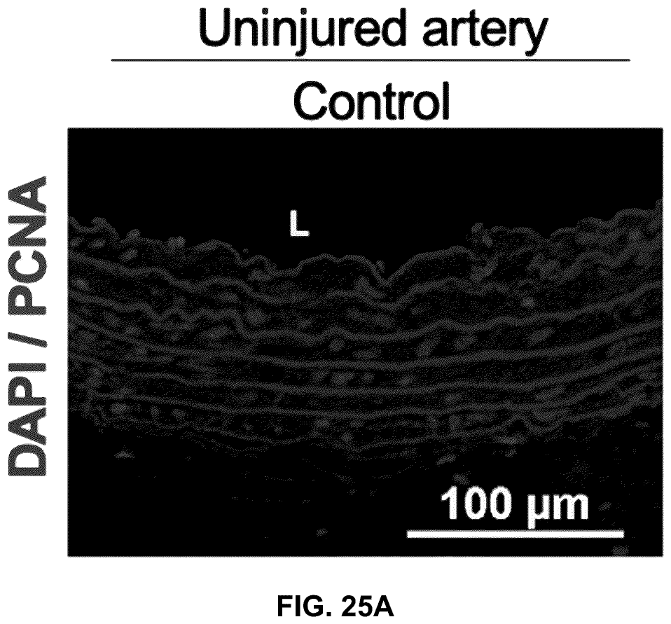

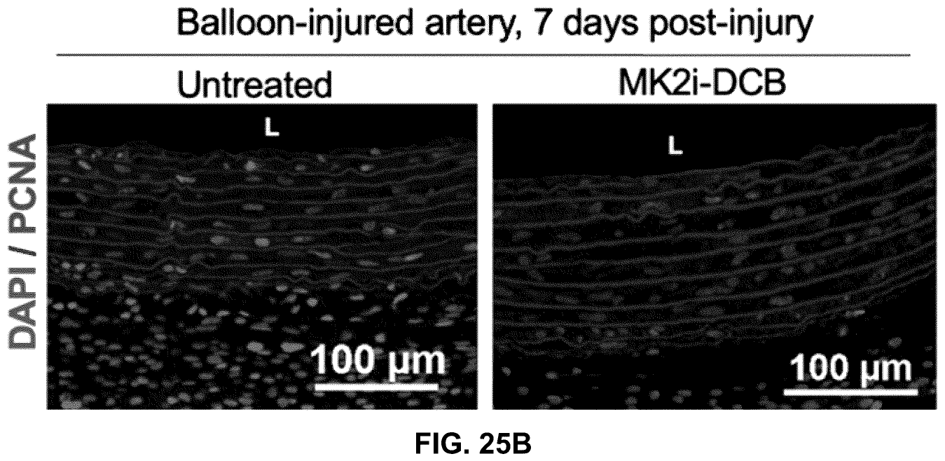

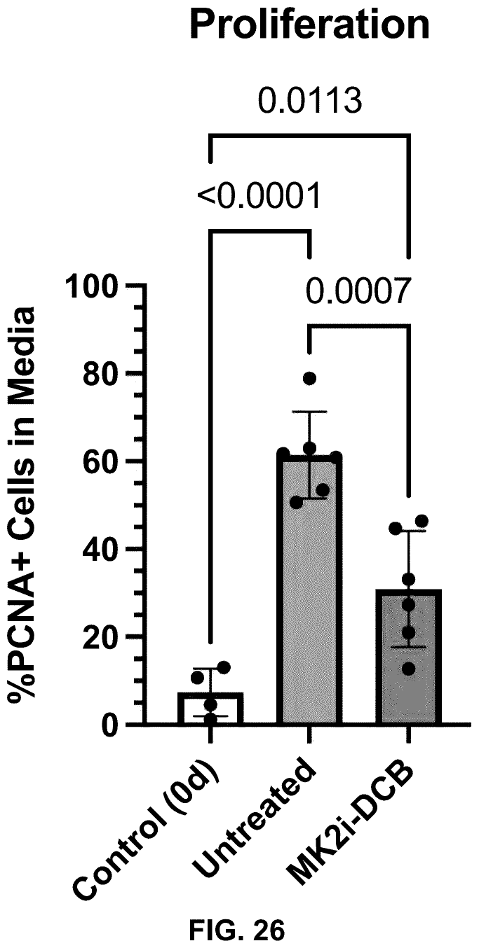

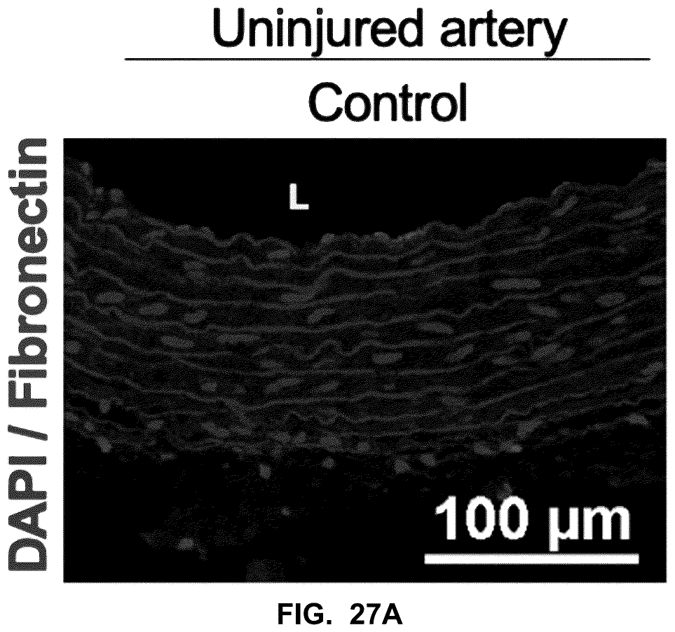

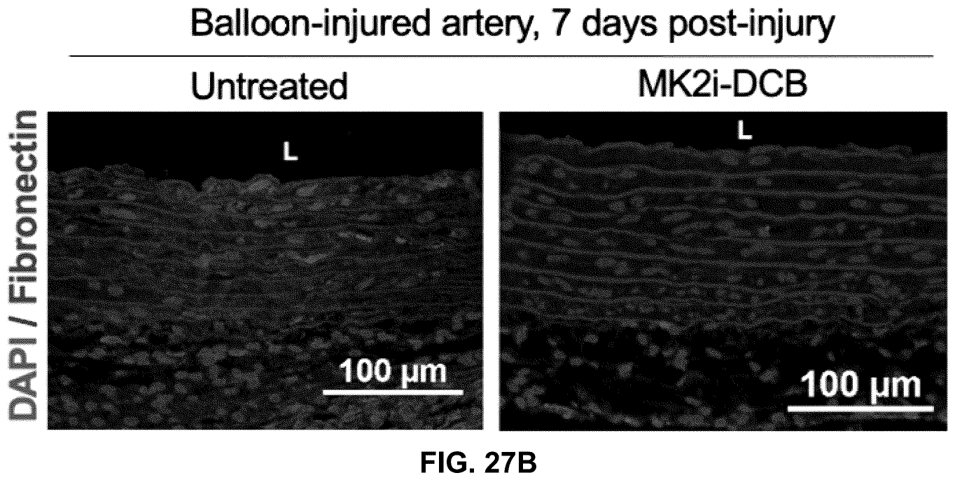

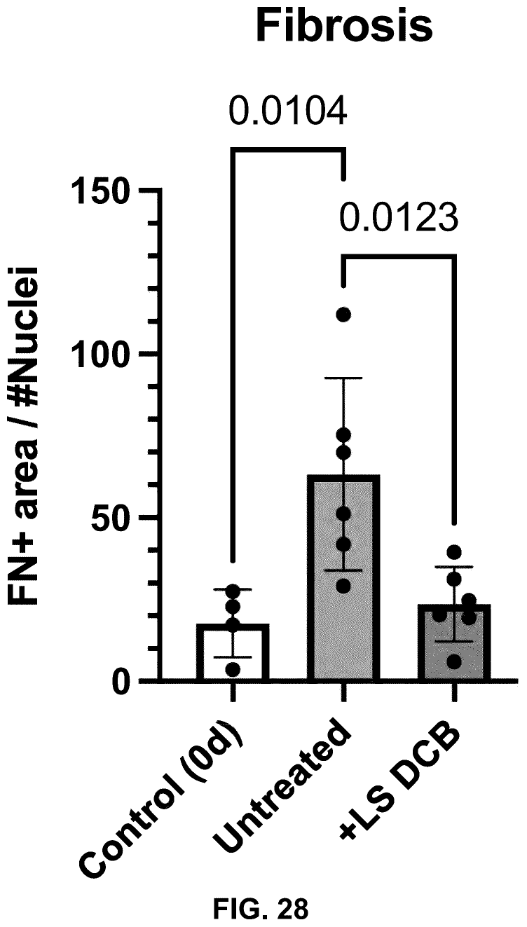

MK2i treatment inhibits proliferation and fibrosis ex vivo: Effects of MK2i treatment were analyzed by incubating rat aortas in the flow loop bioreactor for 7 days. Using PCNA as a marker for proliferating cells, it was observed that balloon damage led to a significant increase in proliferating medial cells after 7 days. MK2i DCB treatment decreased the percentage of PCNA positive cells in the media layer (FIG. 25A, FIG. 25B, and FIG. 26). Balloon damaged aortas expressed significantly more fibronectin throughout the media layer, which is a characteristic of the synthetic smooth muscle cell phenotype, while MK2i DCB treated aortas maintained low expression of fibronectin, similar to fresh, nondamaged tissue samples (FIG. 27A, FIG. 27B, and FIG. 28).

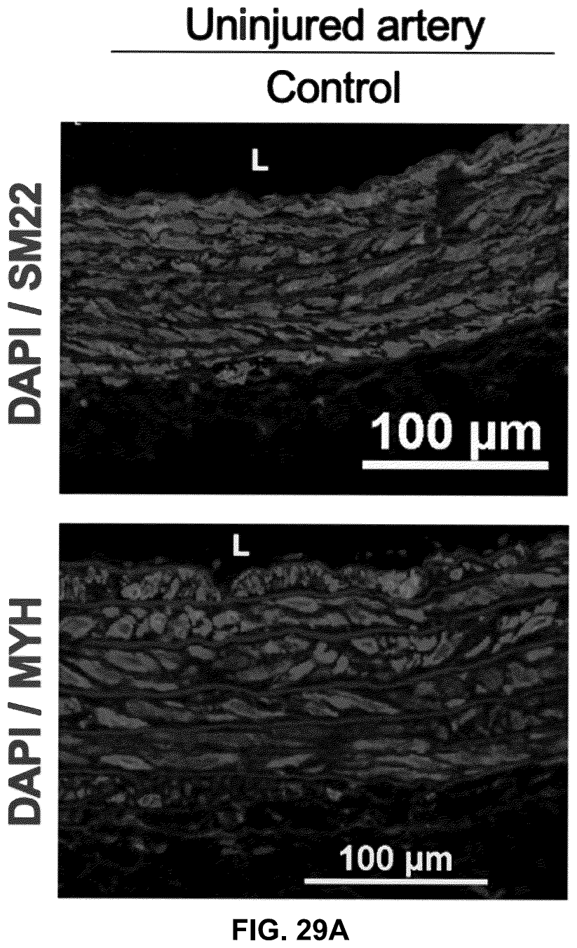

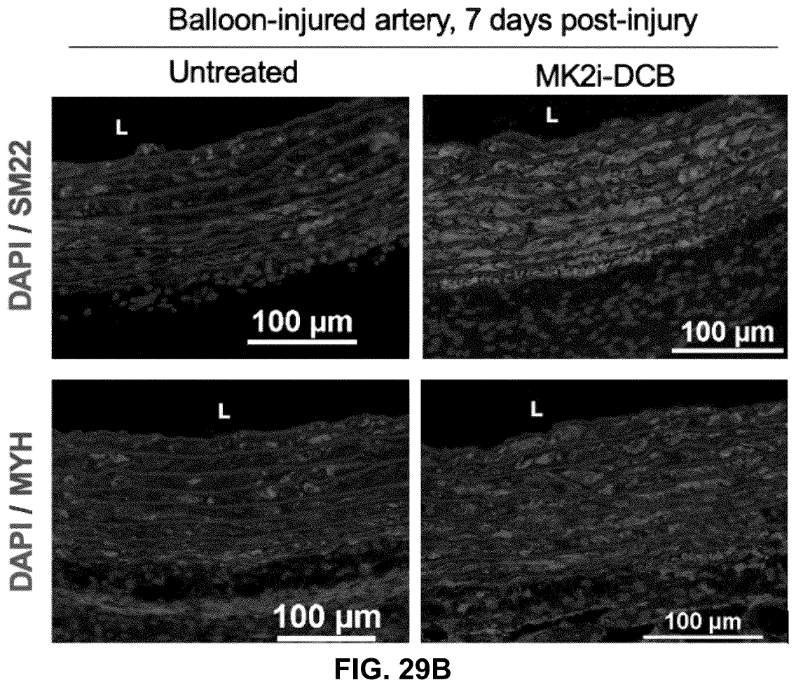

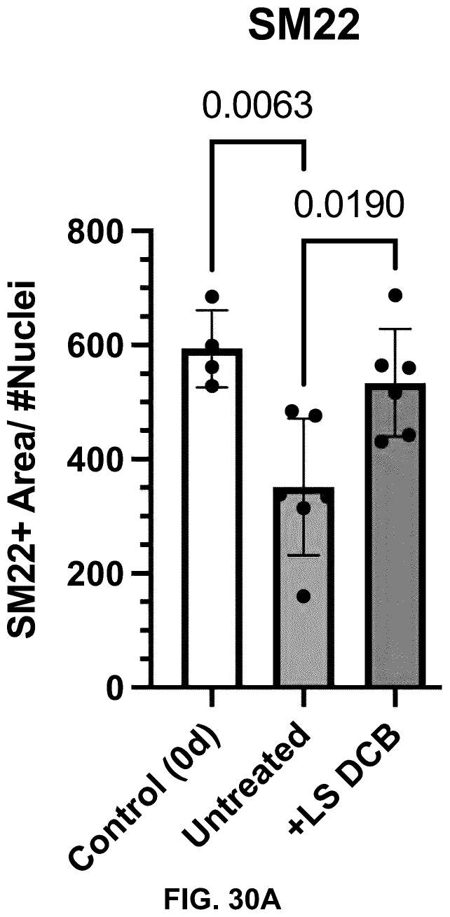

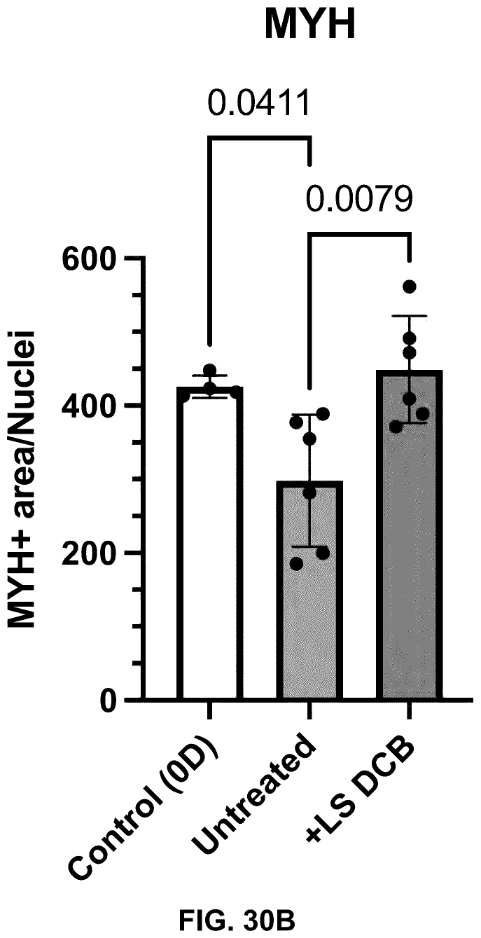

MK2i preserves vessel contractility ex vivo: Preservation of the healthy contractile SMC phenotype is essential to preventing IH development and restenosis after balloon injury. Balloon damage significantly decreased expression of contractile proteins transgelin (SM22) and myosin heavy chain (MYH), while MK2i treated aortas maintained the healthy SMC phenotype by preserving expression of these contractile proteins (FIG. 29A, FIG. 29B, FIG. 30A, and FIG. 30B).

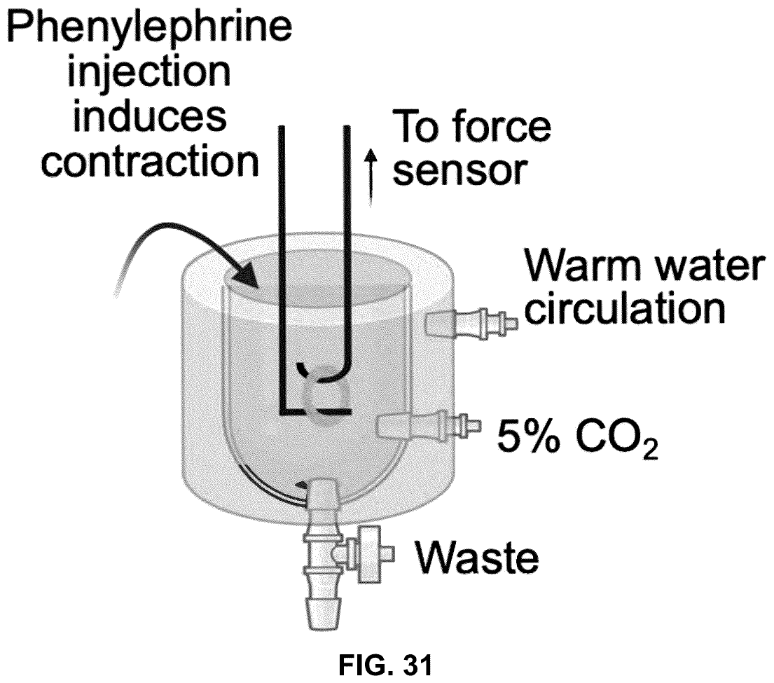

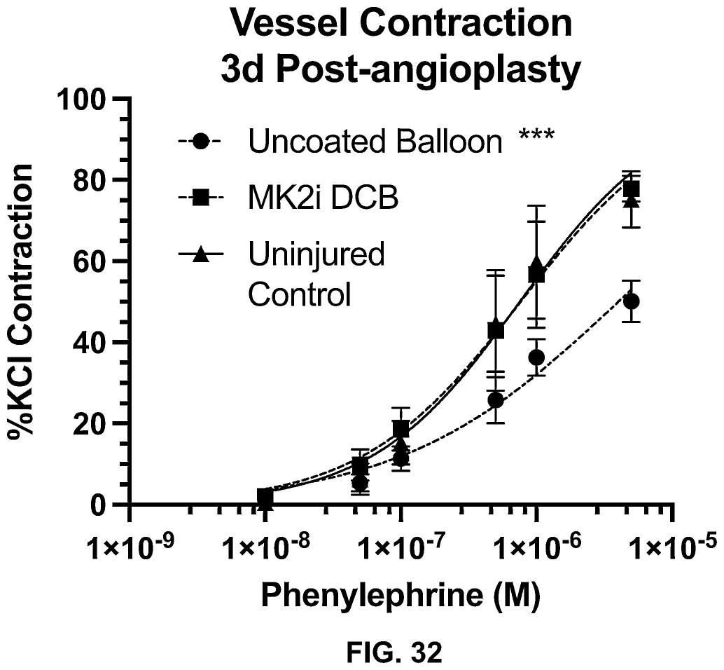

As a functional readout of blocking SMC phenotype switching, the contractile responses of untreated, treated, and damaged aortas to phenylephrine (PE), a SMC specific agonist, were compared after 3 d in the flow loop bioreactor. Aorta segments were hung in a muscle bath (FIG. 31) and treated with a dose curve of PE. FIG. 32 shows that balloon damage in the absence of treatment resulted in a loss of SMC-specific vessel contraction. MK2i DCB treatment preserved SMC contractile force to levels similar to that of the 3 d incubated, untreated, undamaged control arteries. Since the MTT assay confirmed viability, these data suggest that balloon injury leads to loss of contractile function that is not due to cell death. Therefore, MK2i DCB treatment effectively prevent SMCs from switching to the synthetic phenotype and losing contractility.

MK2i DCB treatment blocks IH stenosis in an in vivo rat carotid artery balloon injury model. To assess the ability of MK2i DCBs to block IH development after balloon injury, an in vivo rat carotid artery balloon injury model was employed. Previous studies have shown that rat carotid artery balloon injury consistently results in neointima formation by 14 days after the intervention.

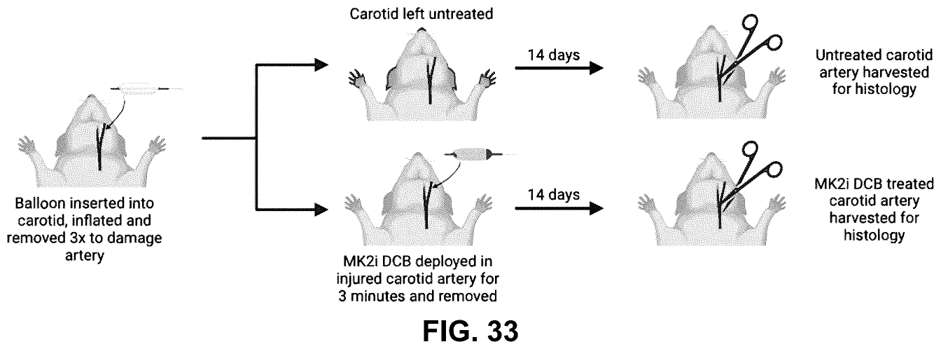

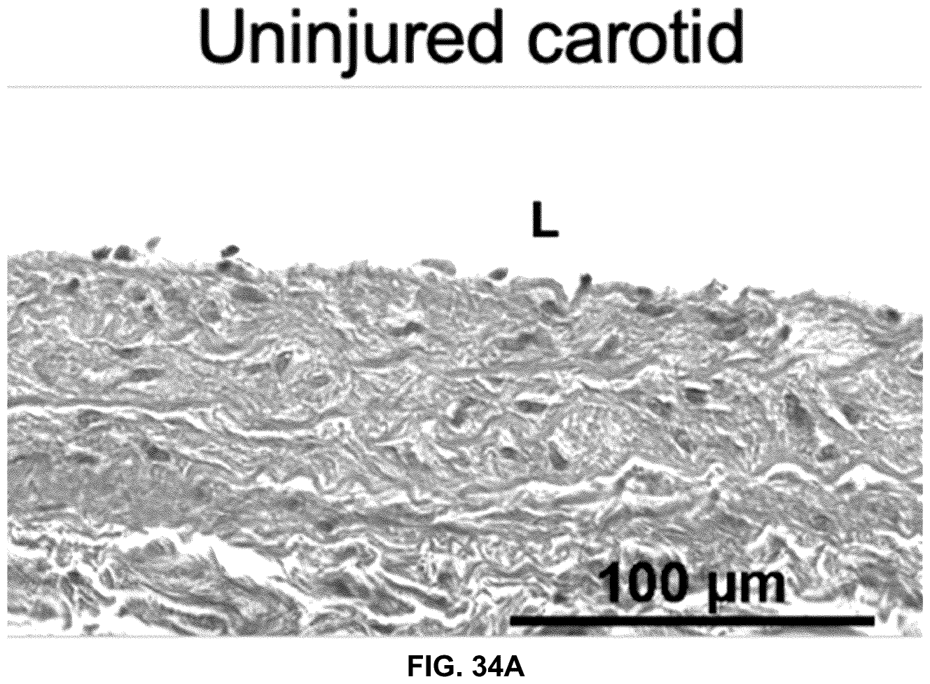

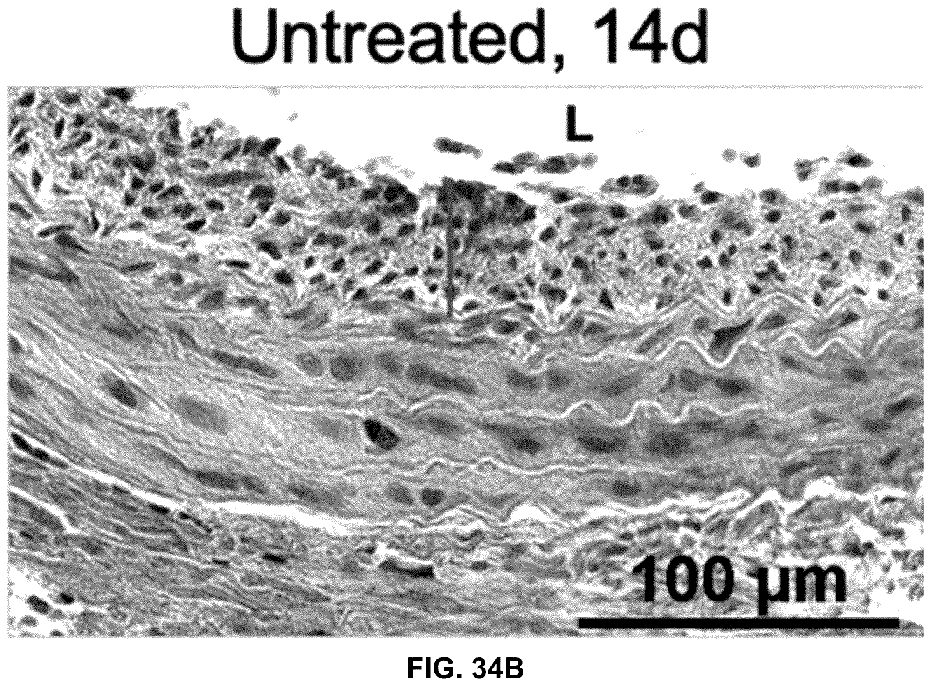

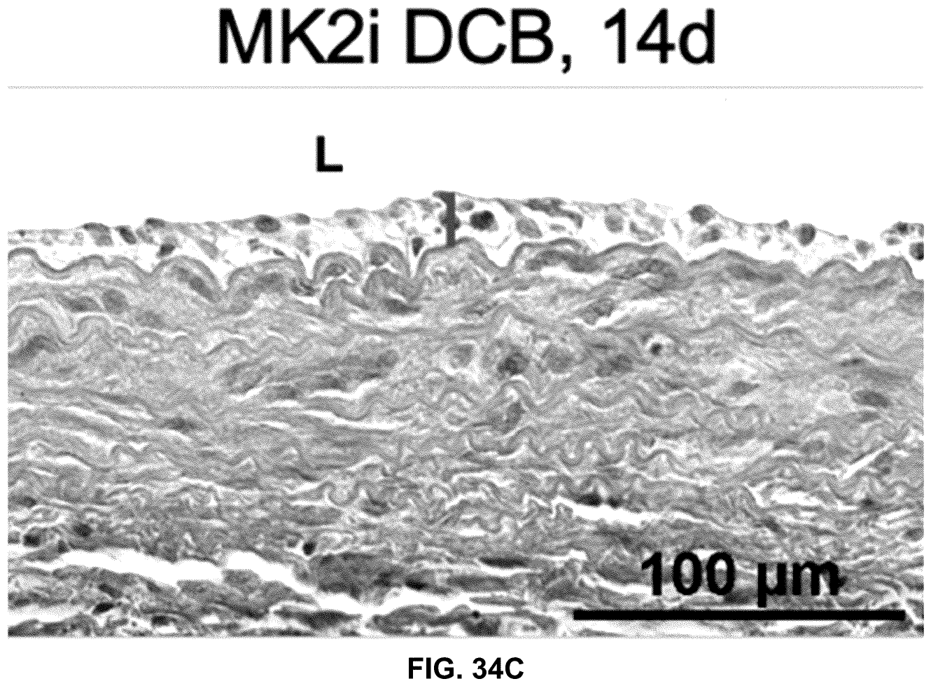

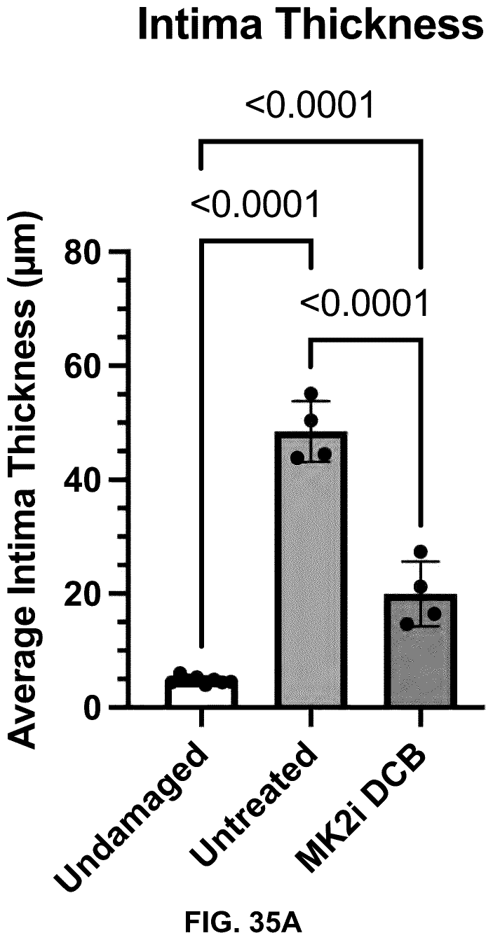

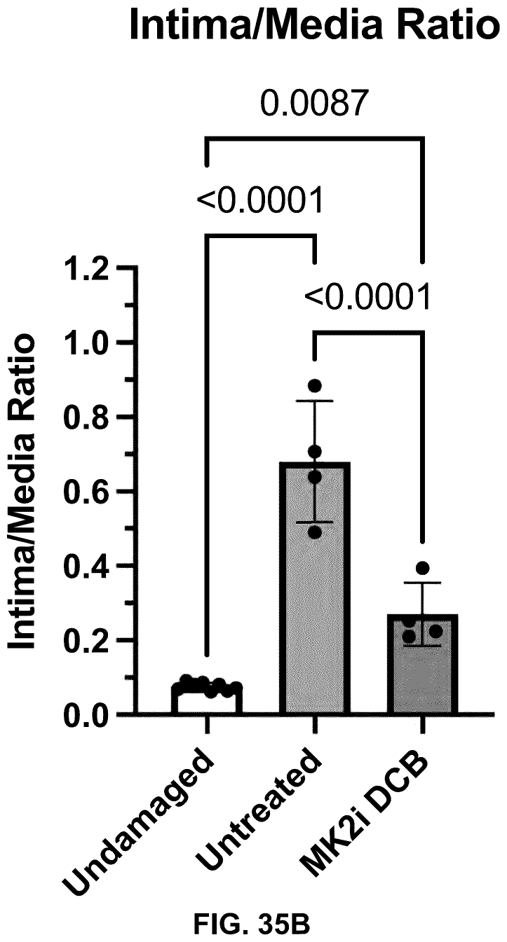

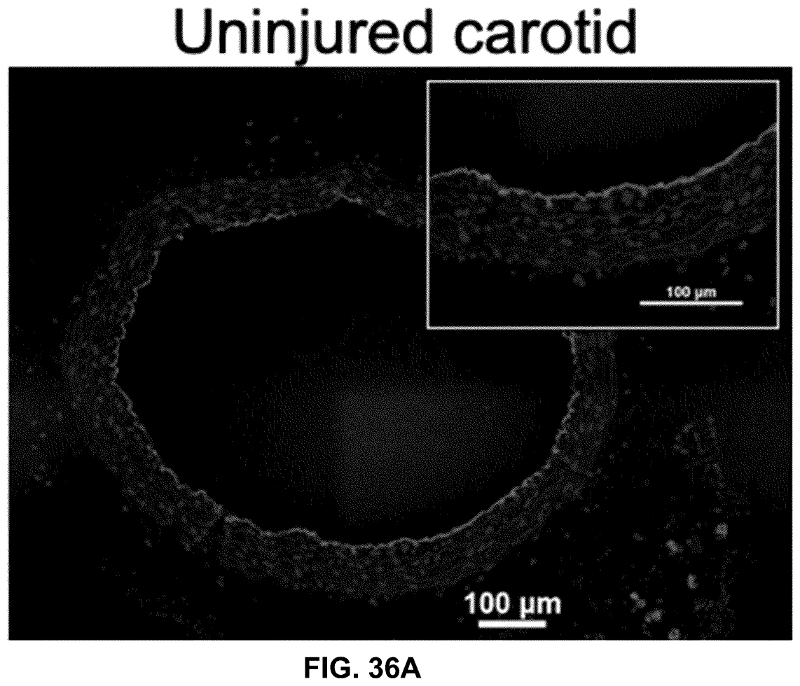

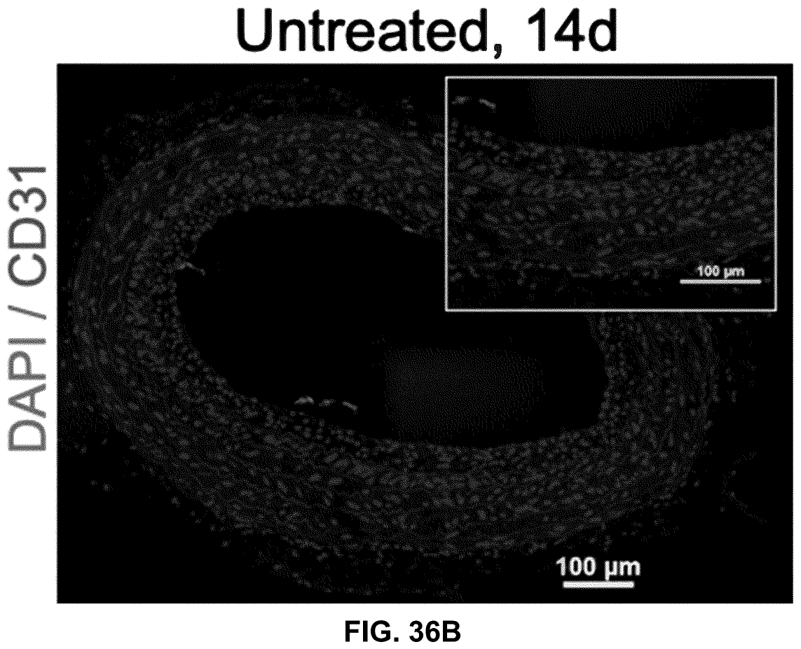

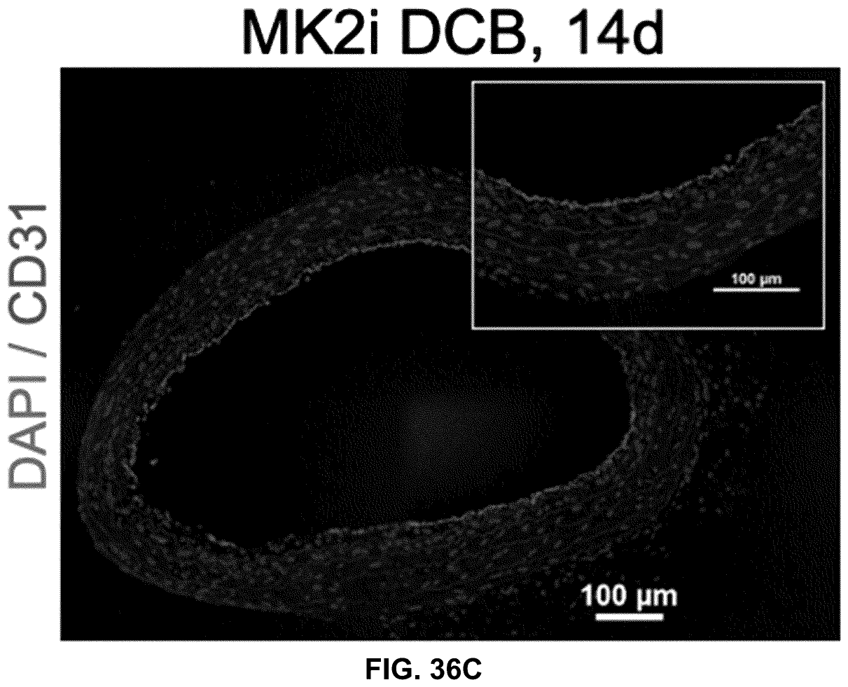

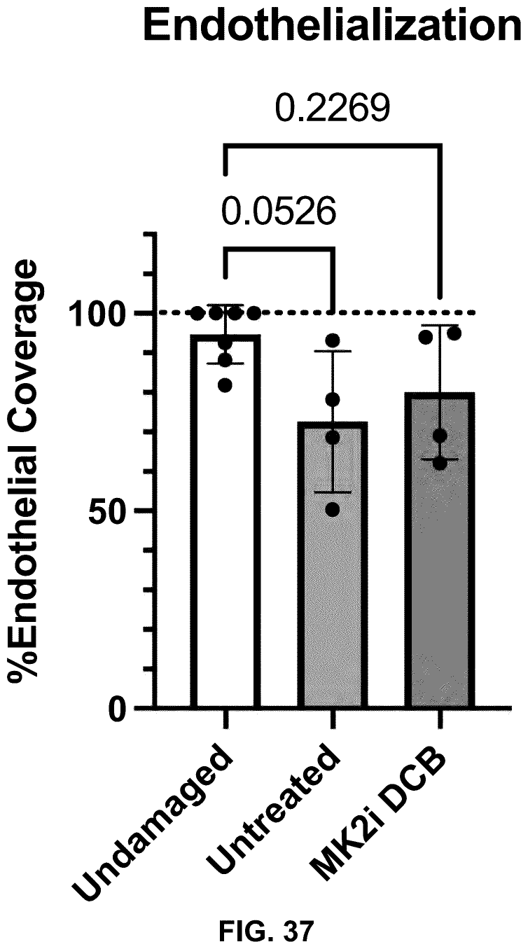

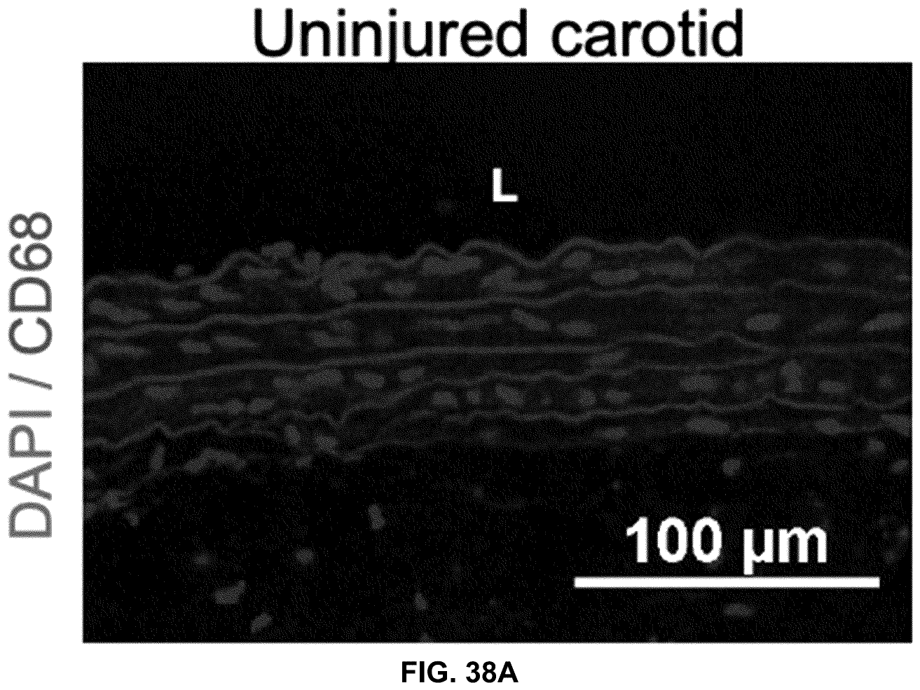

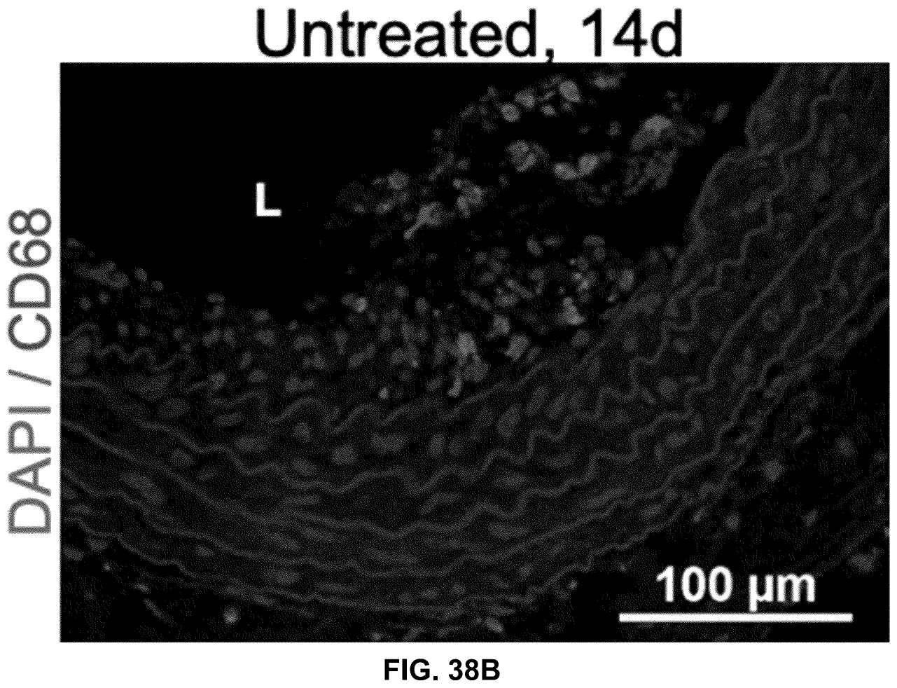

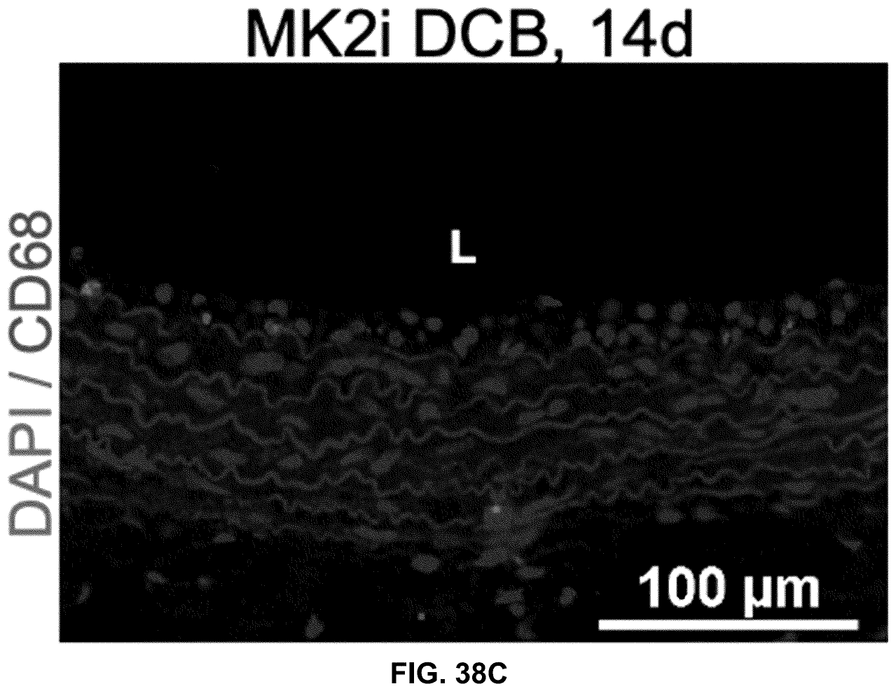

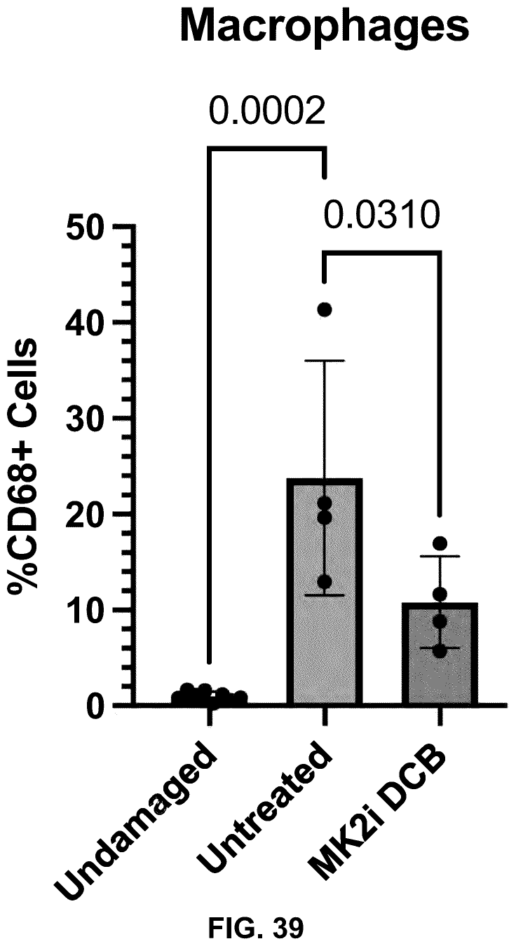

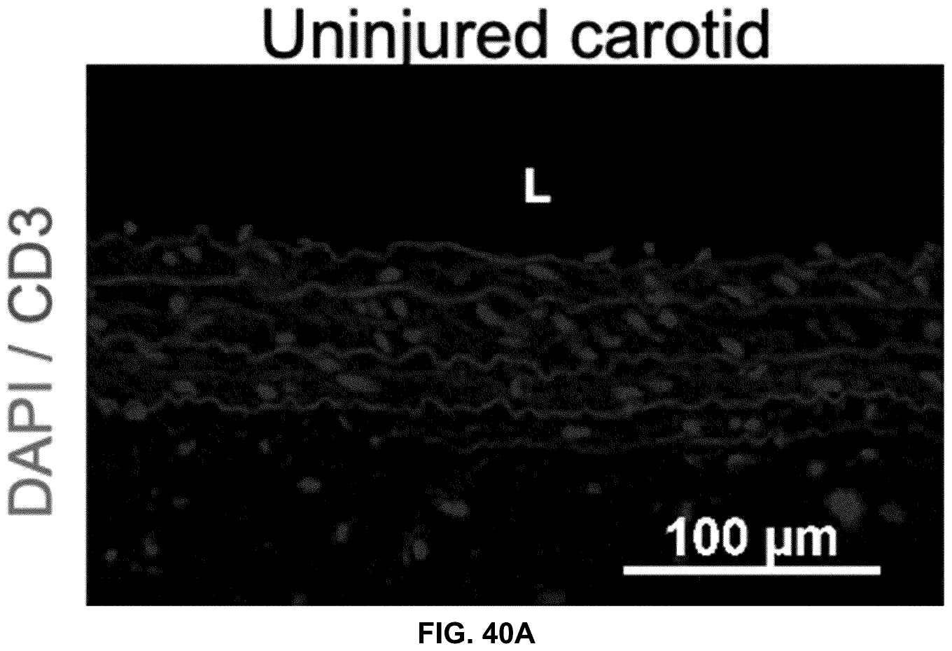

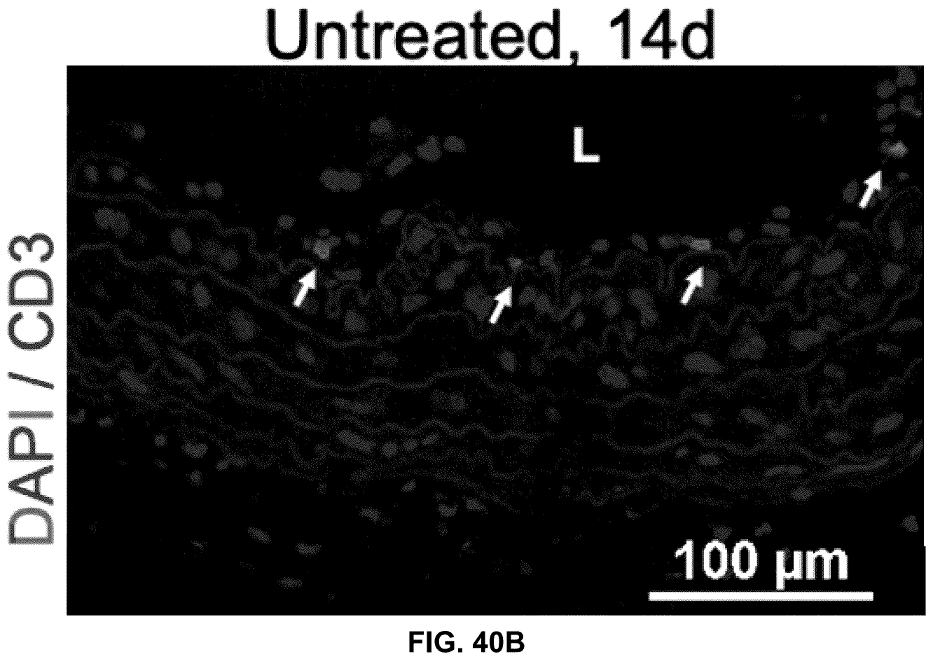

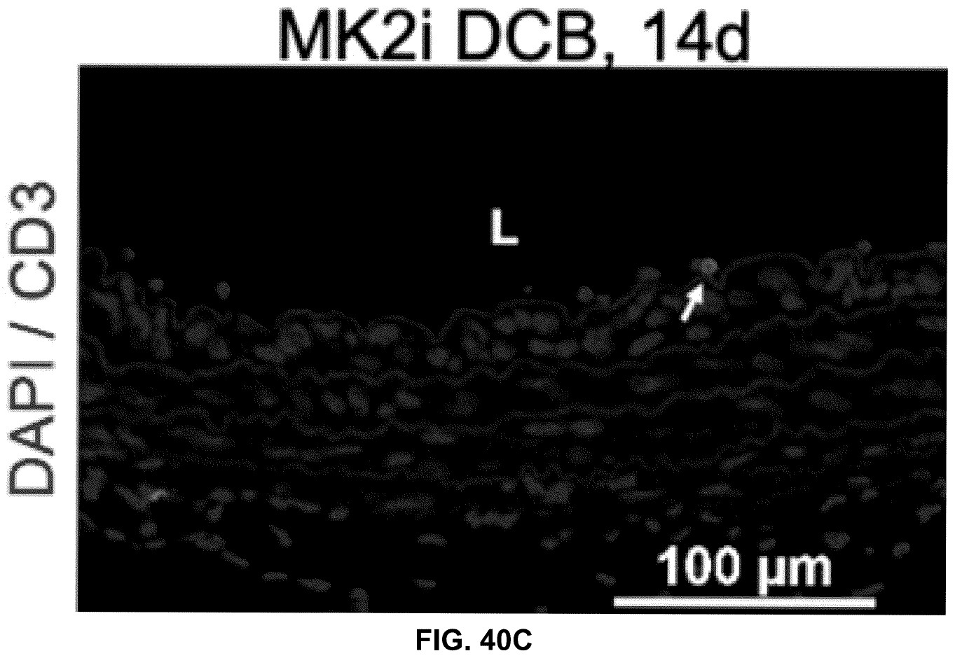

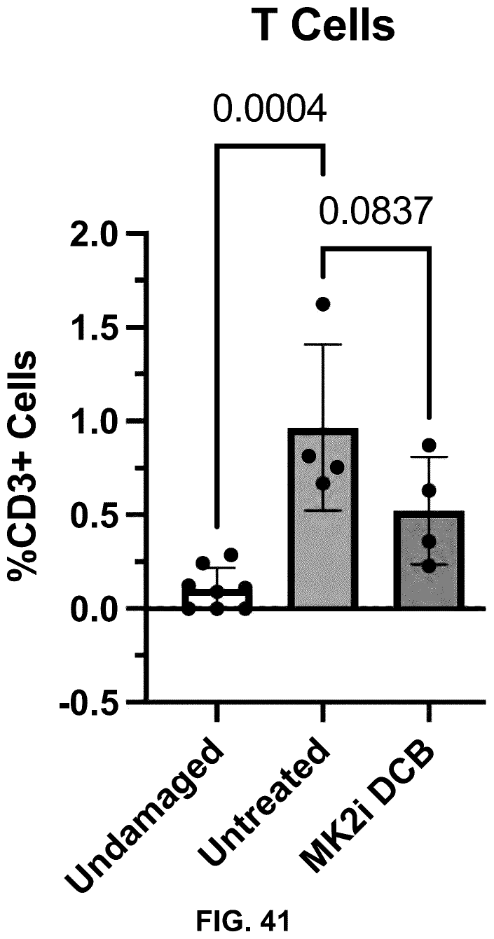

Carotid angioplasty was performed, and arteries were then either left untreated or treated with a follow-up application of the DCB at the site of injury (FIG. 33). Neointima development was then analyzed at 14 days post-surgery. Angioplasty resulted in significant neointima formation, whereas MK2i DCB treatment inhibited IH (FIG. 34, FIG. 34B, and FIG. 34C) and reduced the intima/media ratio (FIG. 35A and FIG. 35B). Importantly, it was observed that MK2i DCB treatment did not impair vessel endothelialization (FIG. 36A, FIG. 36B, FIG. 36C, and FIG. 37) as seen with paclitaxel balloons in other studies. Furthermore, MK2i DCB treatment inhibited macrophage infiltration (FIG. 38A, FIG. 38B, FIG. 38C, and FIG. 39) and T cell recruitment (FIG. 40A, FIG. 40B, FIG. 40C, and FIG. 41) induced by angioplasty, both of which contribute to intimal thickening.

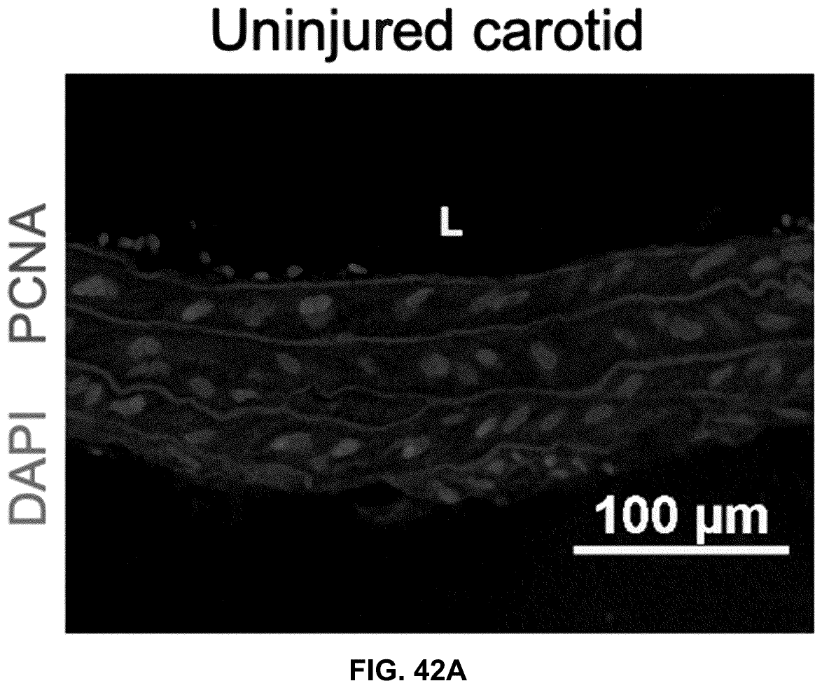

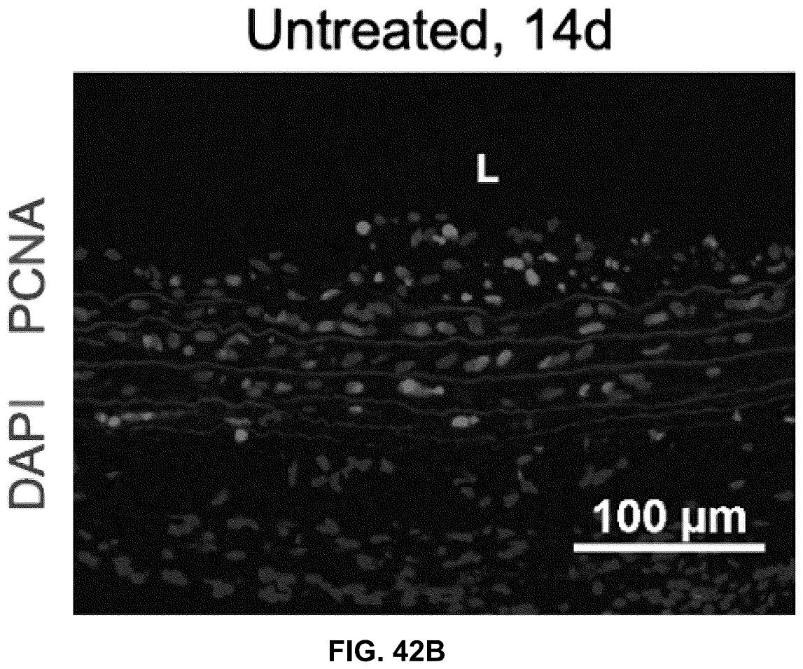

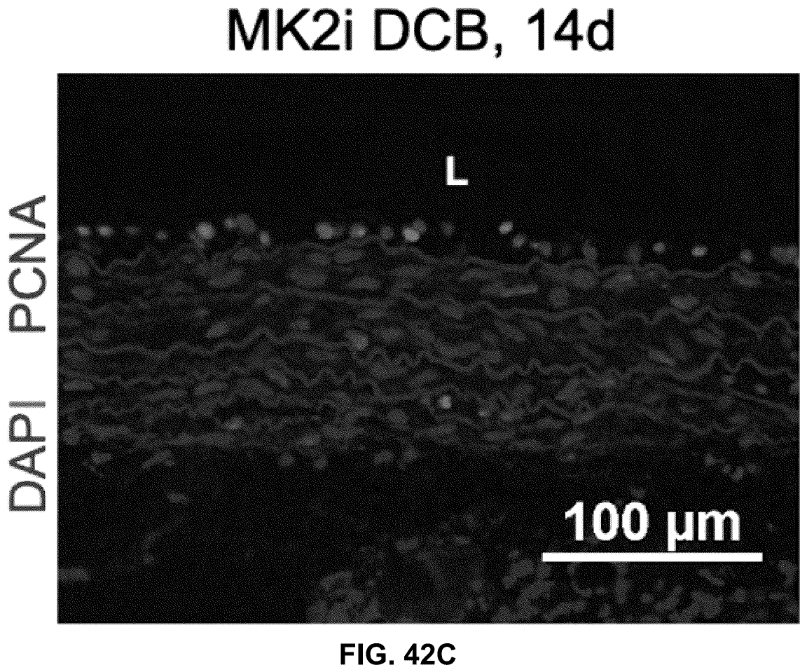

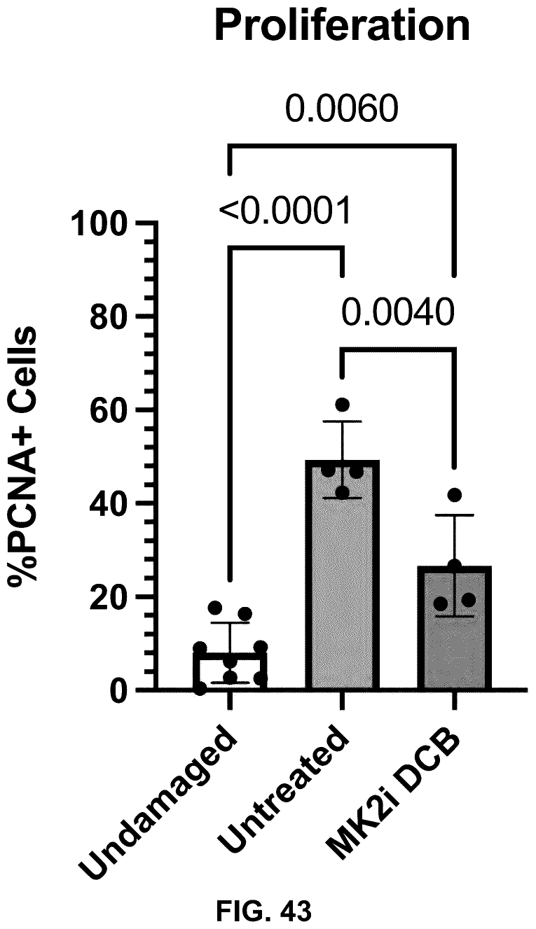

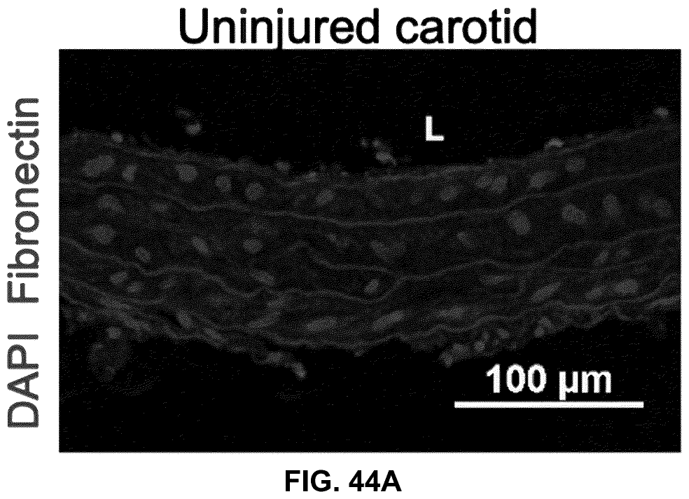

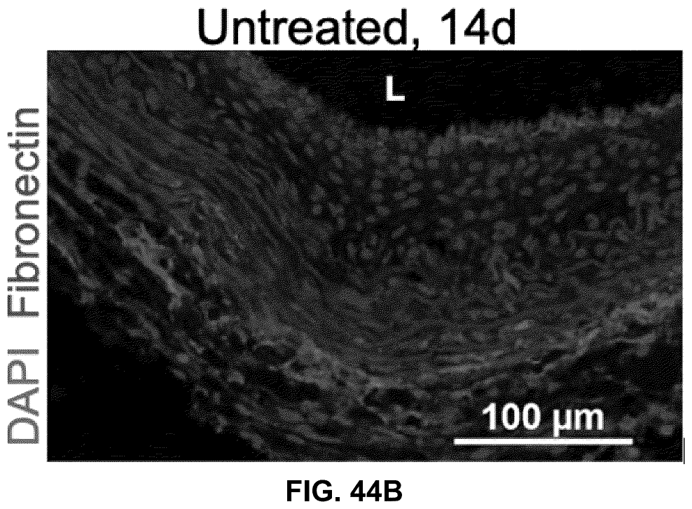

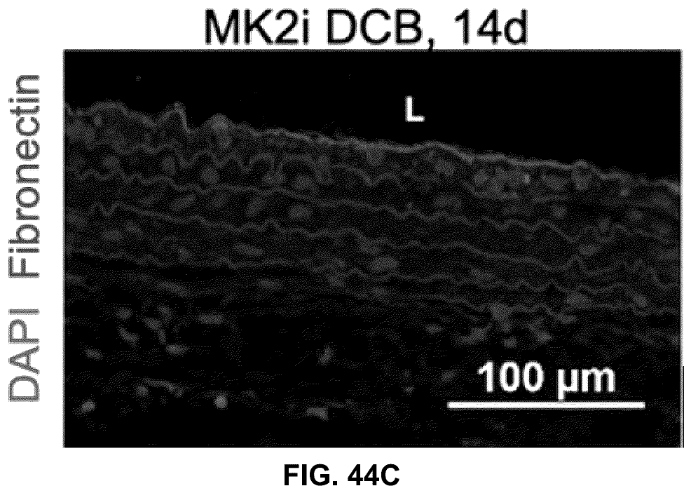

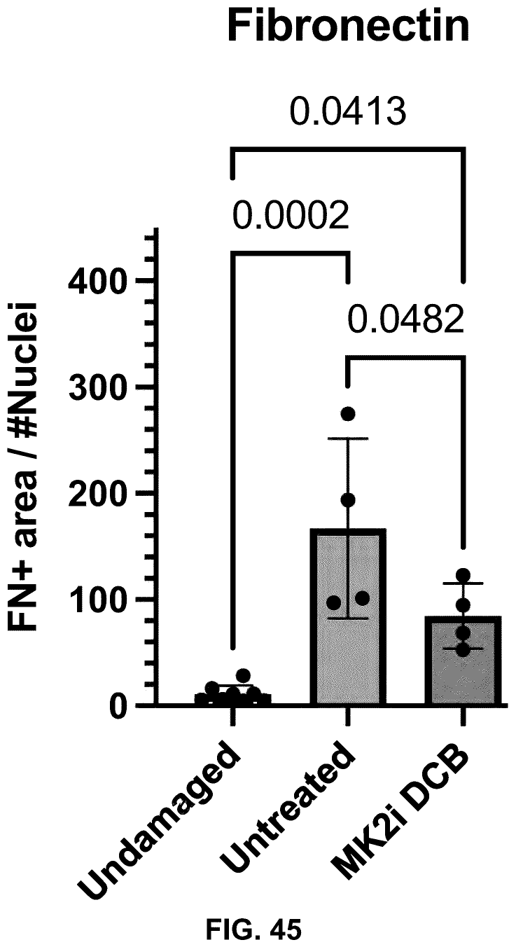

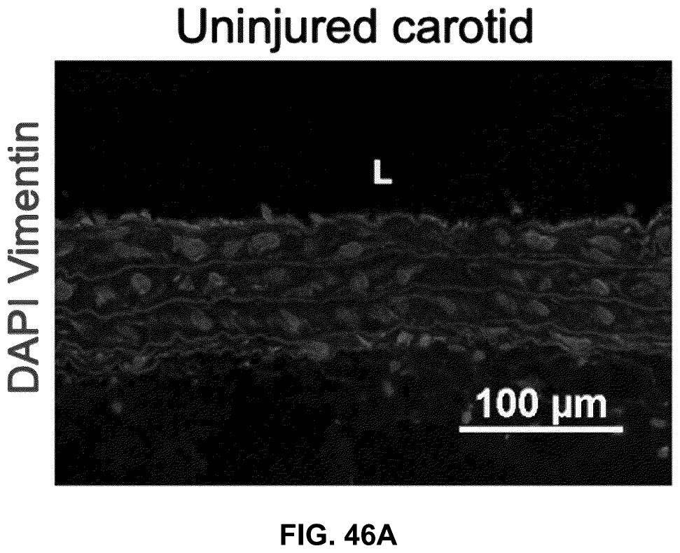

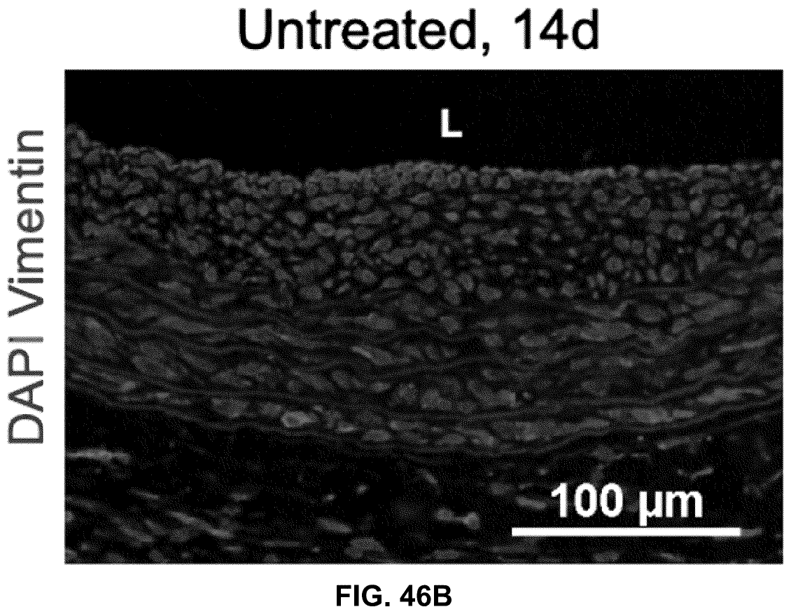

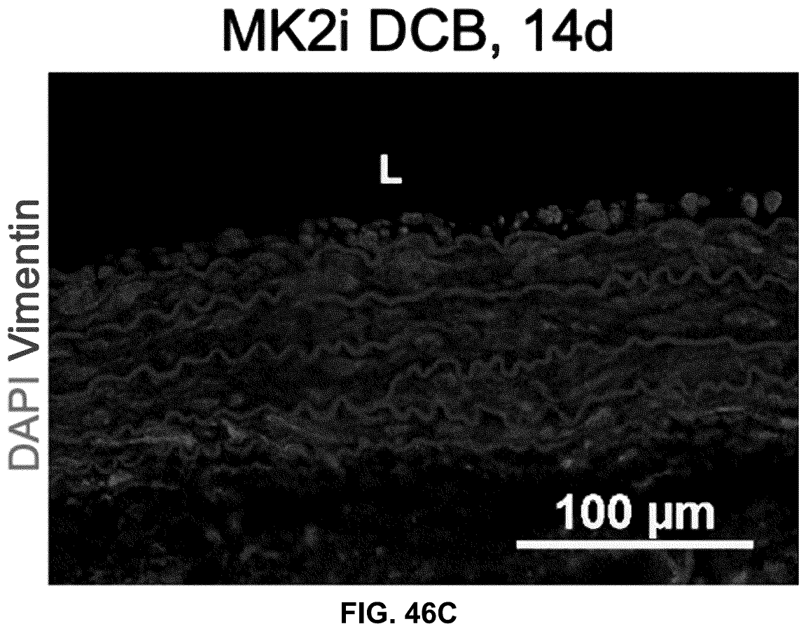

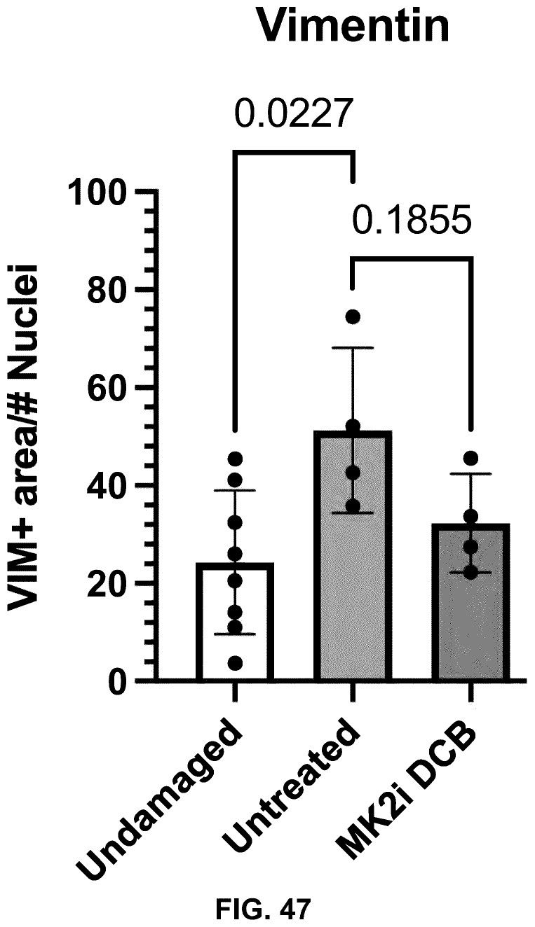

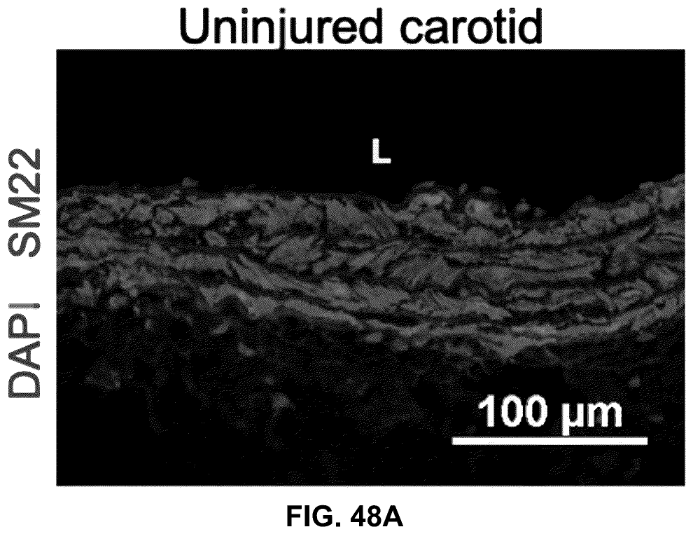

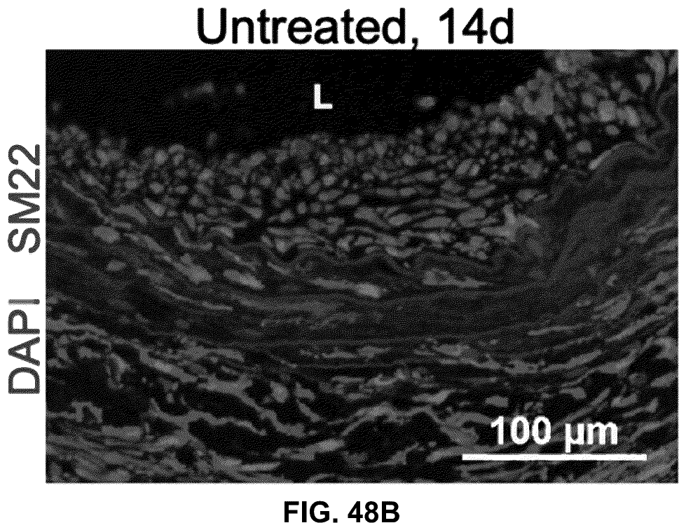

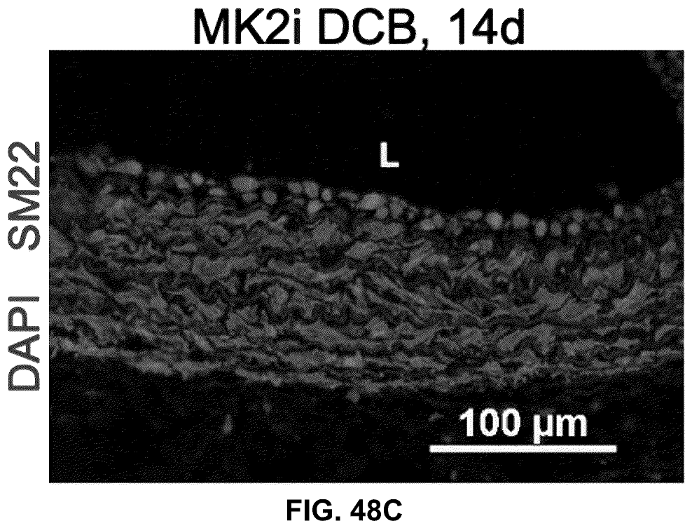

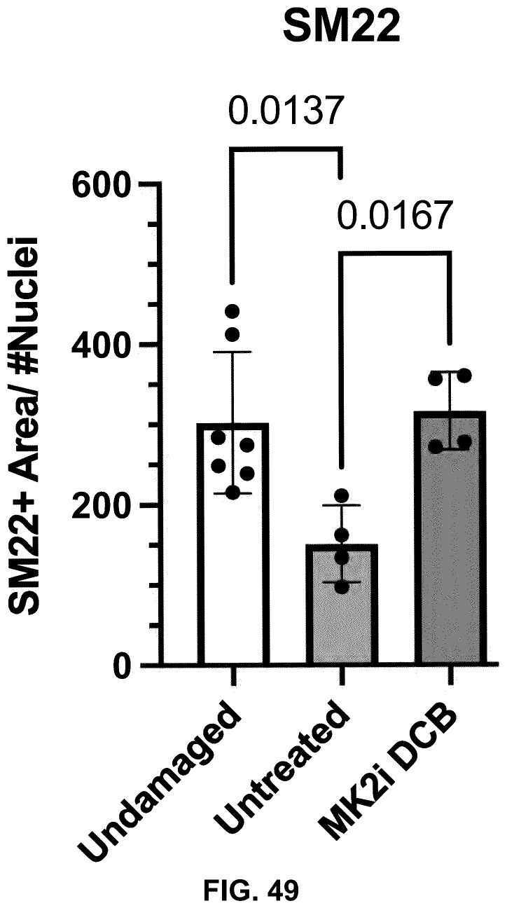

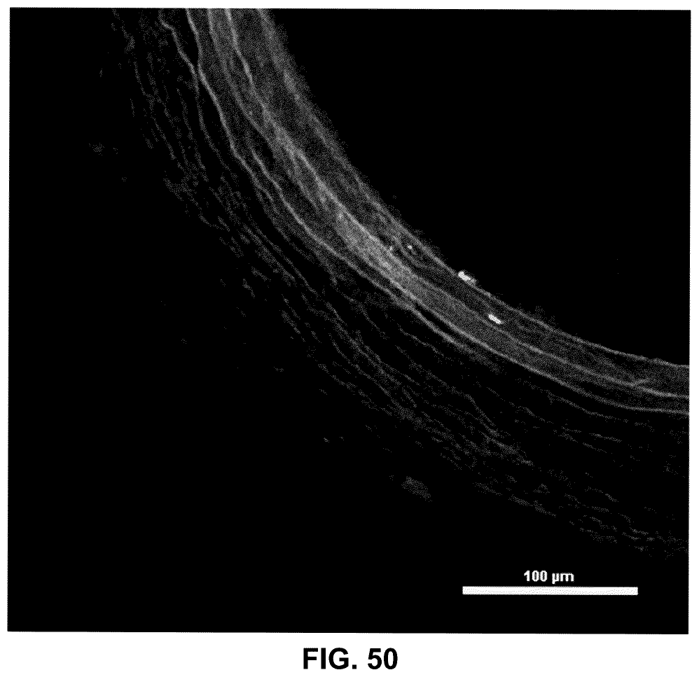

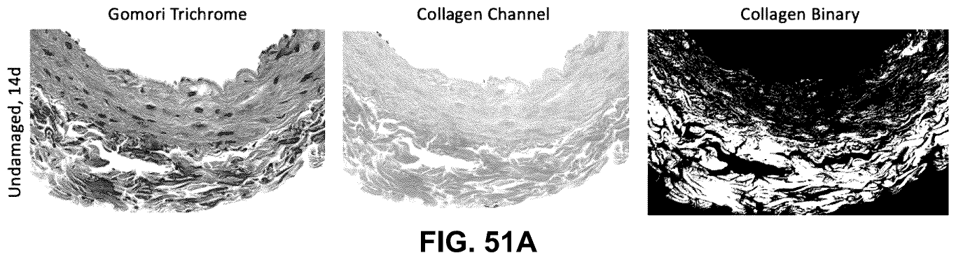

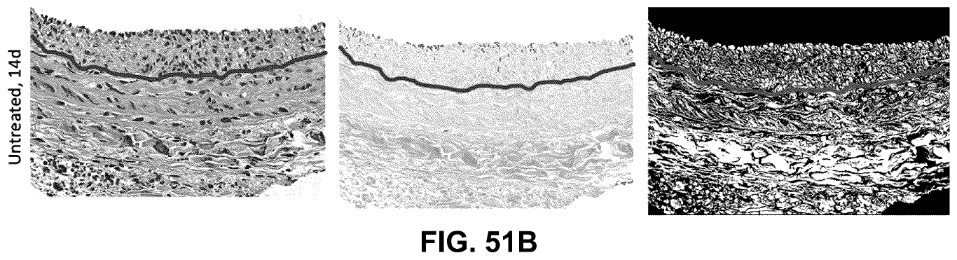

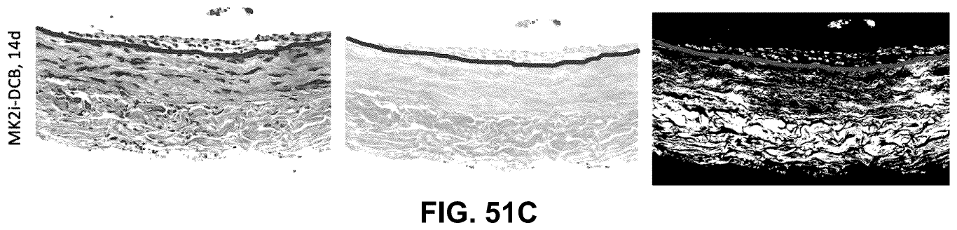

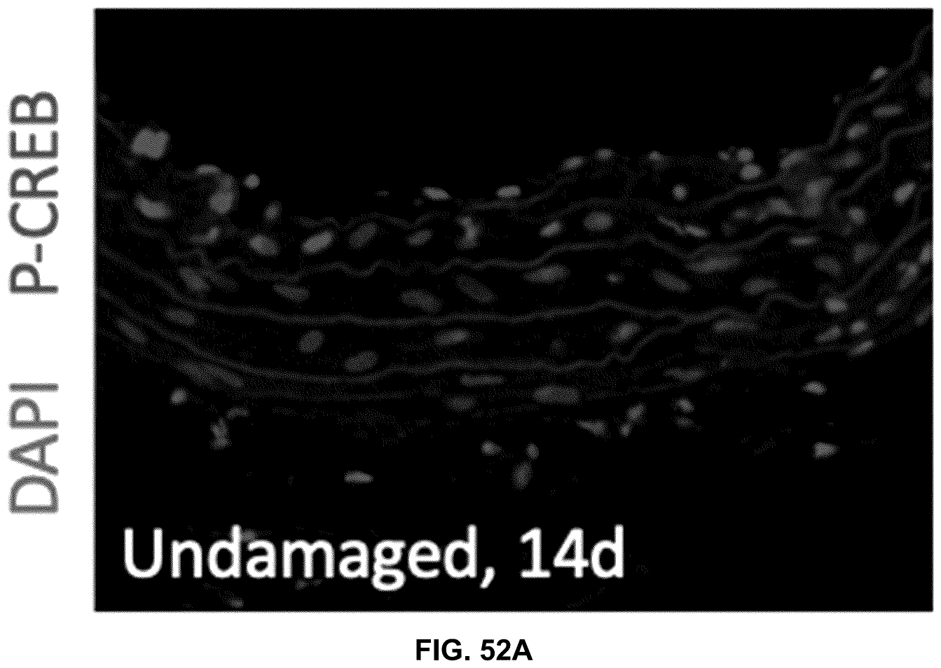

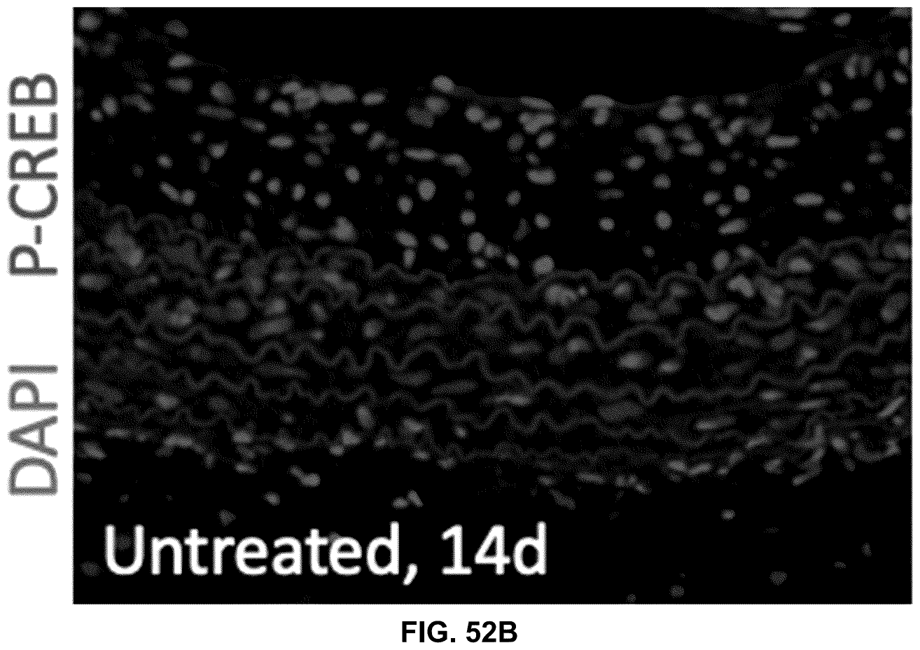

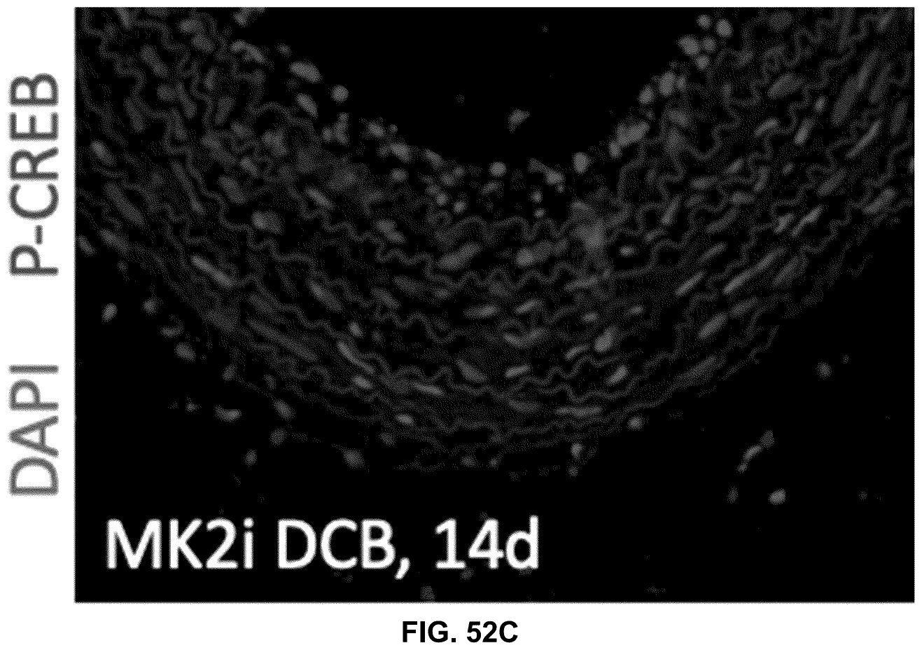

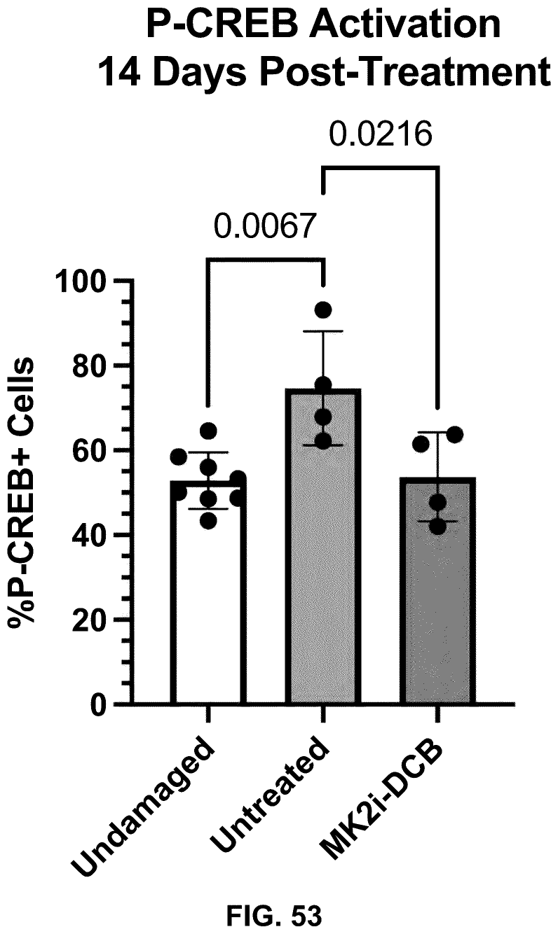

MK2i delivery also blocked SMC phenotype switching at the site of the angioplasty. MK2i DCB treatment inhibited proliferation (FIG. 42A, FIG. 42B, FIG. 42C, and FIG. 43), fibronectin (FIG. 44A, FIG. 44B, FIG. 44C, and FIG. 45) and vimentin (FIG. 46A, FIG. 46B, FIG. 46C, and FIG. 47), all hallmarks of the synthetic phenotype. Conversely, MK2i DCB treatment preserved expression of the contractile phenotype marker SM22 (FIG. 48A, FIG. 48B, FIG. 48C, and FIG. 49). Additionally, neointima layers of untreated arteries had visually denser collagen networks than the neointima of MK2i DCB treated arteries as shown with Masson's trichrome (FIG. 51A, FIG. 51B, and FIG. 51C), indicating MK2i inhibits development of mature IH by preventing SMCs from switching to the ECM-producing synthetic phenotype. This preservation of healthy contractile SMCs is due to continued MK2i retention in the MK2i DCB treated arteries at 14 days after injury (FIG. 50), leading to sustained inhibition of MK2 and lowered levels of P-CREB throughout the treated tissues (FIG. 52A, FIG. 52B, FIG. 52C, and FIG. 53). These results suggest that MK2i DCBs may represent an ideal therapeutic option that blocks SMC phenotype switching, inflammation, and neointima formation, while allowing endothelial repair after angioplasty.

Discussion: Data presented in these studies show that DCB approaches for intravascular delivery of MK2i+PPAA formulations are promising for prevention of restenosis after angioplasty. DCBs achieve successful MK2i delivery to ex vivo aortas (FIG. 14A, FIG. 14B, FIG. 15A, and FIG. 15B) and are retained within the arteries for up to 7 days post-treatment (FIG. 18).

MK2i DCB delivery achieves therapeutically relevant MK2i concentrations within the tissue. Pharmacodynamic studies showed that DCB delivery significantly inhibited CREB phosphorylation at all time points during 7-day incubation in the dynamic culture bioreactor, suggesting that the MK2i bioactivity is sustained in the tissue for at least one week. This longevity of action is important for long-term inhibition of restenosis, as the majority of cellular proliferation occurs within the first week after interventional vascular procedures and inflammation tends to peak around 3 days post-injury. This early proliferation and inflammation coincide with the SMC phenotype switch, and by targeting a node that blocks this switch, it may reduce the ongoing secretion of matrix and neointimal growth that drive restenosis. It was shown that MK2i DCB treatment successfully mitigated hallmark signs of phenotype switching including cell proliferation, fibrosis, and contractile protein loss ex vivo, and preserve vessel contractility 3 days after balloon damage.

MK2i DCB treatment completely blocked vessel fibrosis and fibronectin production and significantly decreased PCNA expression relative to the untreated, balloon damaged arteries. IHC for contractile markers SM22 and MYH also showed that MK2i DCB had a significant effect on maintaining contractile protein expression, with levels similar to healthy control arteries and statistically different from the untreated, balloon damaged arteries.

The delivery profile of the MK2i DCBs show that the majority of the delivered MK2i is initially concentrated near the luminal surface. This spatially co-localizes with the region where SMCs of the vessel wall are more damaged by balloon injury. Shear stress has been shown to be a factor in SMC phenotype switching, so the SMCs adjacent to the lumen may be important to target early after injury and denudation of the endothelium that occurs at the angioplasty site.

The advantages of the MK2i DCBs observed in ex vivo rat aorta studies motivated our focus on this delivery platform for testing in the in vivo rat carotid balloon injury model. There is also a stronger clinical precedent for DCB use than there is for convective delivery approaches. Treatment with the MK2i DCB effectively inhibited neointima formation in damaged rat carotids after 14 days, whereas untreated balloon damaged arteries had noticeable formation of a thick neointima in all samples. Additionally, MK2i DCBs blocked cellular proliferation in medial SMCs, reduced immune cell infiltration, and moderated vessel fibrosis, while preserving the contractile SMC phenotype within the media layer of the arteries. Vessels treated with MK2i DCB were also re-endothelialized, suggesting that the arteries reached a healthy steady state condition post-angioplasty. Early re-endothelialization may also contribute to the reduction in IH observed in the treated vessels. In sum, these data suggest that MK2i DCB's treatment is associated with multiple mechanisms that prevent IH and promote vessel patency.

Current DCBs in use in the clinic employ proliferation-targeting drugs such as paclitaxel and sirolimus. These drugs can be effective in preventing restenosis over naked balloon angioplasty, especially in coronary artery disease, but still have issues with primary patency and amputation at 1 year in peripheral artery restenosis suggesting a need for improved balloon treatments in the peripheral arterial circulation. This is believed to be at least partially due to the limitations of applying drugs that only target cellular proliferation. Proliferation-targeting drugs also inhibit vessel re-endothelialization, which is important for preventing thrombosis and IH, and fail to inhibit the underlying signaling pathways and SMC phenotype switch that drive IH progression. On the other hand, MK2 inhibition reduces inflammation, ECM production, and SMC phenotype switching without inhibiting re-endothelialization, all of the key facets known to contribute to neointima formation and popular targets for current anti-restenosis research. Previous RNA sequencing data on primary human coronary artery SMCs showed that MK2 inhibition leads to broad changes in genetic expression that maintain the SMC contractile phenotype. MK2i inhibition resulted in a significant decrease in many proinflammatory genes, leading to decreased inflammatory cell recruitment seen here in vivo, and maintenance of multiple genes associated with contractile proteins, thus preserving SMC contractility and expression of contractile phenotype proteins. Through these vast genetic changes, MK2i prevents IH formation supporting MK2i DCB treatment as a more holistic approach to restenosis prevention than current DCBs.

It is understood that the foregoing detailed description and accompanying examples are merely illustrative and are not to be taken as limitations upon the scope of the technology.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use, may be made without departing from the spirit and scope thereof.

For reasons of completeness, various aspects of the disclosure are set out in the following numbered clauses:

Clause 1. A drug coated medical device comprising: a medical device having an outer surface; and a multi-layer drug coating on at least a portion of the outer surface, the multi-layer drug coating comprising at least one polyelectrolyte bilayer, the polyelectrolyte bilayer comprising a polycationic layer including a cationic therapeutic, and a polyanionic layer capable of electrostatically binding to the polycationic layer, the polyanionic layer including an anionic polymer having recurring units of formula (I)

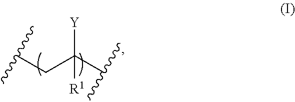

wherein Y is —C(O)OH, —SO₃H, or —PO₃H₂, and R¹ is C₁-C₁₂ alkyl.

Clause 2. The drug coated medical device of clause 1, wherein the multi-layer drug coating comprises a saccharide.

Clause 3. The drug coated medical device of clause 2, wherein the polycationic layer, the polyanionic layer, or both comprise the saccharide.

Clause 4. The drug coated medical device of clause 2 or 3, wherein the saccharide comprises a monosaccharide, a disaccharide, a trisaccharide, or a combination thereof.

Clause 5. The drug coated medical device of any one of clauses 2-4, wherein the saccharide comprises lactosucrose, trehalose, or sucrose.

Clause 6. The drug coated medical device of any one of clauses 2-5, wherein the saccharide comprises lactosucrose.

Clause 7. The drug coated medical device of any one of clauses 1-6, wherein the cationic therapeutic comprises a cationic peptide, a cationic conjugate, a cationic oligonucleotide analog, a cationic nanoparticle, or a combination thereof.

Clause 8. The drug coated medical device of any one of clauses 1-7, wherein the cationic therapeutic is a cationic peptide.

Clause 9. The drug coated medical device of clause 8, wherein the cationic peptide comprises a p38 pathway inhibitory peptide, a cell penetrating peptide, or a combination thereof.

Clause 10. The drug coated medical device of clause 8 or 9, wherein the cationic peptide comprises an amino acid sequence selected from the group consisting of SEQ ID NO:1, SEQ ID NO:2, SEQ ID NO:3, SEQ ID NO:4, and SEQ ID NO:5.

Clause 11. The drug coated medical device of any one of clauses 1-10, wherein the multi-layer drug coating comprises the cationic therapeutic at about 0.5 μg/cm² to about 10 μg/cm².

Clause 12. The drug coated medical device of any one of clauses 1-11, wherein the recurring units of formula (I) are of formula (I-a)

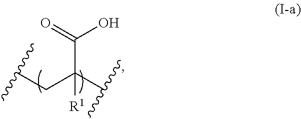

Wherein R¹ is C₁-C₆ alkyl.

Clause 13. The drug coated medical device of any one of clauses 1-12, wherein the anionic polymer has a pKa of about 6 to about 7.4.

Clause 14. The drug coated medical device of any one of clauses 1-13, wherein the anionic polymer has a number average molecular weight of about 10 kDa to about 40 kDa as measured by gel permeation chromatography.

Clause 15. The drug coated medical device of any one of clauses 1-14, wherein the multi-layer drug coating comprises less than 15 polyelectrolyte bilayers.

Clause 16. The drug coated medical device of any one of clauses 1-15, wherein the multi-layer drug coating comprises 2 to 8 polycationic layers.

Clause 17. The drug coated medical device of any one of clauses 1-16, wherein the multi-layer drug coating comprises 2 to 8 polyanionic layers.

Clause 18. The drug coated medical device of any one of clauses 1-17, wherein the multi-layer drug coating has a thickness of about 0.5 μm to about 4 μm.

Clause 19. The drug coated medical device of any one of clauses 1-18, further comprising a base layer between the outer surface of the medical device and the multi-layer drug coating, wherein the base layer comprises an aminosilane.

Clause 20. The drug coated medical device of any one of clauses 1-19, wherein the medical device comprises an angioplasty balloon catheter, a stent, a vascular graft, a surgical tool, a wound dressing, a syringe, a pacemaker, or a prosthetic.

Clause 21. The drug coated medical device of any one of clauses 1-20, wherein the medical device comprises an angioplasty balloon catheter, a stent, or a vascular graft.

Clause 22. The drug coated medical device of any one of clauses 1-21, wherein the medical device is an angioplasty balloon catheter comprising: a catheter; and an inflatable balloon coupled to the catheter, wherein the outer surface is an outer surface of the inflatable balloon.

Clause 23. A method of making a drug coated medical device, the method comprising: (a) immersing an outer surface of a medical device into a first mixture, the first mixture comprising a first solvent and an anionic polymer having recurring units of formula (I)

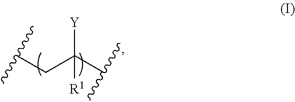

wherein Y is —C(O)OH, —SO₃H, or —PO₃H₂, and R¹ is C₁-C₁₂ alkyl, to provide a polyanionic layer on the outer surface of the medical device, wherein the polyanionic layer includes the anionic polymer; (b) immersing the outer surface of the medical device into a second mixture, the second mixture comprising a second solvent and a cationic therapeutic, to provide a polycationic layer on a surface of the polyanionic layer, wherein the polycationic layer includes the cationic therapeutic; and (c) optionally repeating steps (a) and (b).

Clause 24. The method of clause 23, wherein the first mixture, the second mixture, or both comprise a saccharide.

Clause 25. The method of clause 24, wherein the saccharide comprises lactosucrose, trehalose, or sucrose.

Clause 26. The method of clause 24 or 25, wherein the first mixture, the second mixture, or both comprise the saccharide at about 50 mM to about 150 mM.

Clause 27. The method of any one of clauses 23-26, wherein the first mixture comprises the anionic polymer at about 5 μM to about 10 μM.

Clause 28. The method of any one of clauses 23-27, wherein the second mixture comprises the cationic therapeutic at about 50 μM to about 100 μM.

Clause 29. The method of any one of clauses 23-28, wherein the outer surface of the medical device is capable of electrostatically binding to the cationic therapeutic or the anionic polymer.

Clause 30. The method of any one of clauses 23-29, wherein the outer surface of the medical device is plasma treated, silane treated, or both prior to step (a).

Clause 31. The method of any one of clauses 23-30, wherein the first solvent and the second solvent are each independently an alcohol.

Clause 32. A method of treating a vascular disease in a subject in need thereof, the method comprising implanting the drug coated medical device of any one of clauses 1-22 at a vascular site of the subject.

Clause 33. The method of clause 32, wherein the vascular disease comprises intimal hyperplasia, peripheral artery disease, atherosclerosis, arteriovenous fistula stenosis, coronary artery disease, or a combination thereof.

Clause 34. The method of clause 32 or 33, wherein the method reduces the risk of restenosis in the subject.

Clause 35. The method of any one of clauses 32-34, wherein the vascular site is a blood vessel, a vein graft, a synthetic graft, or an arteriovenous fistula.

Clause 36. A method of delivering a cationic peptide to a subject in need thereof, the method comprising: (a) implanting the drug coated medical device of clause 22 at a vascular site of the subject; (b) inflating the balloon of the medical device, thereby transferring a portion of the cationic peptide from the medical device to the vascular site; (c) deflating the balloon of the medical device; (d) optionally repeating steps (b) and (c); and (e) removing the drug coated medical device from the vascular site.

Clause 37. The method of clause 36, wherein at least 2%, by weight, of the cationic therapeutic is transferred from the balloon to the vascular site following removal of the medical device.

Sequences

(SEQ ID NO: 1) MK2i: YARAAARQARAKALARQLGVAA
(SEQ ID NO: 2) R6-MK2i: RRRRRR-KALARQLGVAA
(SEQ ID NO: 3) TAT-MK2i: GRKKRRQRRRPPQ-KALARQLGVAA
(SEQ ID NO: 4) Penetratin-MK2i: RQIKIWFQNRRMKWKK-KALARQLGVAA
(SEQ ID NO: 5) Transportan-MK2i: GWTLNSAGYLLGKINLKALAALAKKIL-KALARQLGVAA

What is claimed is:

1. A drug coated medical device comprising:
a medical device having an outer surface; and
a multi-layer drug coating on at least a portion of the outer surface, the multi-layer drug coating comprising at least one polyelectrolyte bilayer, the polyelectrolyte bilayer comprising
a polycationic layer including a cationic therapeutic, and
a polyanionic layer capable of electrostatically binding to the polycationic layer, the polyanionic layer including an anionic polymer having recurring units of formula (I) 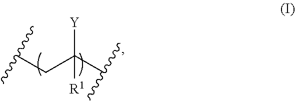
wherein
Y is —C(O)OH, —SO₃H, or —PO₃H₂, and
R¹ is C₁-C₁₂ alkyl.

2. The drug coated medical device of claim 1, wherein the multi-layer drug coating comprises a saccharide.

3. The drug coated medical device of claim 2, wherein the polycationic layer, the polyanionic layer, or both comprises the saccharide.

4. The drug coated medical device of claim 2, wherein the saccharide comprises a monosaccharide, a disaccharide, a trisaccharide, or a combination thereof.

5. The drug coated medical device of claim 2, wherein the saccharide comprises lactosucrose, trehalose, or sucrose.

6. The drug coated medical device of claim 2, wherein the saccharide comprises lactosucrose.

7. The drug coated medical device of claim 1, wherein the cationic therapeutic comprises a cationic peptide, a cationic conjugate, a cationic oligonucleotide analog, a cationic nanoparticle, or a combination thereof.

8. The drug coated medical device of claim 1, wherein the cationic therapeutic is a cationic peptide.

9. The drug coated medical device of claim 8, wherein the cationic peptide comprises a p38 pathway inhibitory peptide, a cell penetrating peptide, or a combination thereof.

10. The drug coated medical device of claim 8, wherein the cationic peptide comprises an amino acid sequence selected from the group consisting of SEQ ID NO:1, SEQ ID NO:2, SEQ ID NO: 3, SEQ ID NO:4, and SEQ ID NO:5.

11. The drug coated medical device of claim 1, wherein the multi-layer drug coating comprises the cationic therapeutic at about 0.5 μg/cm² to about 10 μg/cm².

12. The drug coated medical device of claim 1, wherein the recurring units of formula (I) are of formula (I-a) 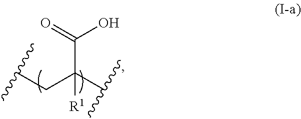
wherein
R¹ is C₁-C₆ alkyl.

13. The drug coated medical device of claim 1, wherein the anionic polymer has a pKa of about 6 to about 7.4.

14. The drug coated medical device of claim 1, wherein the anionic polymer has a number average molecular weight of about 10 kDa to about 40 kDa as measured by gel permeation chromatography.

15. The drug coated medical device of claim 1, wherein the multi-layer drug coating comprises less than 15 polyelectrolyte bilayers.

16. The drug coated medical device of claim 1, wherein the multi-layer drug coating comprises 2 to 8 polycationic layers.

17. The drug coated medical device of claim 1, wherein the multi-layer drug coating comprises 2 to 8 polyanionic layers.

18. The drug coated medical device of claim 1, wherein the multi-layer drug coating has a thickness of about 0.5 μm to about 4 μm.

19. The drug coated medical device of claim 1, further comprising a base layer between the outer surface of the medical device and the multi-layer drug coating, wherein the base layer comprises an aminosilane.

20. The drug coated medical device of claim 1, wherein the medical device comprises an angioplasty balloon catheter, a stent, a vascular graft, a surgical tool, a wound dressing, a syringe, a pacemaker, or a prosthetic.

21. The drug coated medical device of claim 1, wherein the medical device comprises an angioplasty balloon catheter, a stent, or a vascular graft.

22. The drug coated medical device of claim 1, wherein the medical device is an angioplasty balloon catheter comprising:
a catheter; and
an inflatable balloon coupled to the catheter, wherein the outer surface is an outer surface of the inflatable balloon.

23. A method of making a drug coated medical device, the method comprising:
(a) immersing an outer surface of a medical device into a first mixture, the first mixture comprising a first solvent and an anionic polymer having recurring units of formula (I) 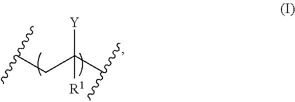
wherein Y is —C(O)OH, —SO₃H, or —PO₃H₂, and R¹ is C₁-C₁₂ alkyl, to provide a polyanionic layer on the outer surface of the medical device, wherein the polyanionic layer includes the anionic polymer;
(b) immersing the outer surface of the medical device into a second mixture, the second mixture comprising a second solvent and a cationic therapeutic, to provide a polycationic layer on a surface of the polyanionic layer, wherein the polycationic layer includes the cationic therapeutic; and
(c) optionally repeating steps (a) and (b).

24. The method of claim 23, wherein the first mixture, the second mixture, or both comprise a saccharide.

25. The method of claim 24, wherein the saccharide comprises lactosucrose, trehalose, or sucrose.

26. The method of claim 24, wherein the first mixture, the second mixture, or both comprise the saccharide at about 50 mM to about 150 mM.

27. The method of claim 23, wherein the first mixture comprises the anionic polymer at about 5 μM to about 10 μM.

28. The method of claim 23, wherein the second mixture comprises the cationic therapeutic at about 50 μM to about 100 μM.

29. The method of claim 23, wherein the outer surface of the medical device is capable of electrostatically binding to the cationic therapeutic or the anionic polymer.

30. The method of claim 23, wherein the outer surface of the medical device is plasma treated, silane treated, or both prior to step (a).

31. The method of claim 30, wherein the first solvent and the second solvent are each individually an alcohol.

32. A method of treating a vascular disease in a subject in need thereof, the method comprising implanting the drug coated medical device of claim 1 at a vascular site of the subject.

33. The method of claim 32, wherein the vascular disease comprises intimal hyperplasia, peripheral artery disease, atherosclerosis, arteriovenous fistula stenosis, coronary artery disease, or a combination thereof.

34. The method of claim 32, wherein the method reduces the risk of restenosis in the subject.

35. The method of claim 32, wherein the vascular site is a blood vessel, a vein graft, a synthetic graft, or an arteriovenous fistula.

36. A method of delivering a cationic peptide to a subject in need thereof, the method comprising:
(a) implanting the drug coated medical device of claim 22 at a vascular site of the subject;
(b) inflating the balloon of the medical device, thereby transferring a portion of the cationic therapeutic from the medical device to the vascular site;
(c) deflating the balloon of the medical device;
(d) optionally repeating steps (b) and (c); and
(e) removing the drug coated medical device from the vascular site.

37. The method of claim 36, wherein at least 2%, by weight, of the cationic therapeutic is transferred from the balloon to the vascular site following removal of the medical device.
